SEQUENCE LISTING (71 sequences)

SEQ ID NO: 1 - DNA, 28 bp, Artificial Sequence
  Description of Artificial Sequence Synthetic primer
taacaaacatgctgattttgacacatgg

SEQ ID NO: 2 - DNA, 20 bp, Artificial Sequence
  Description of Artificial Sequence Synthetic primer
ccaggcacgacaaaacccac

SEQ ID NO: 3 - DNA, 30 bp, Artificial Sequence
  Description of Artificial Sequence Synthetic primer
ctaaatcacccattcagtacatcgatatcg

SEQ ID NO: 4 - DNA, 34 bp, Artificial Sequence
  Description of Artificial Sequence Synthetic primer
cttcatagaacgaacaacgcactacaagactacc

SEQ ID NO: 5 - DNA, 23 bp, Artificial Sequence
  Description of Artificial Sequence Synthetic primer
gaattgtgcgtggatgaggctgg

SEQ ID NO: 6 - DNA, 22 bp, Artificial Sequence
  Description of Artificial Sequence Synthetic primer
caacacgaacgtcatgatactc

SEQ ID NO: 7 - RNA, 20 nt, Artificial Sequence
  Description of Artificial Sequence Synthetic oligonucleotide
auuaauuguaaaaggugaac

SEQ ID NO: 8 - RNA, 20 nt, Artificial Sequence
  Description of Artificial Sequence Synthetic oligonucleotide
auuaauuguaaaagguaaac

SEQ ID NO: 9 - DNA, 20 bp, Severe acute respiratory syndrome coronavirus 2
attaattgtaaaaggtaaac

SEQ ID NO: 10 - DNA, 20 bp, Severe acute respiratory syndrome coronavirus 2
attaattgtaaaaggtgaac

SEQ ID NO: 11 - DNA, 23 bp, Severe acute respiratory syndrome coronavirus 2
attaattgtaaaaggtaaacagg

SEQ ID NO: 12 - DNA, 23 bp, Severe acute respiratory syndrome coronavirus 2
attaattgtaaaaggtgaacagg

SEQ ID NO: 13 - DNA, 184 bp, Artificial Sequence
  Description of Artificial Sequence Synthetic polynucleotide
gtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaa

SEQ ID NO: 14 - RNA, 120 nt, Artificial Sequence
  Description of Artificial Sequence Synthetic polynucleotide
uaacaaacaugcugauuuugacacaugguuuagccagcguggugguaguuauacuaaugacaaagcuugcccauugauugcugcagucauaacaagagaaguggguuuugucgugccugg

SEQ ID NO: 15 - DNA, 175 bp, Artificial Sequence
  Description of Artificial Sequence Synthetic polynucleotide
gaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttg

SEQ ID NO: 16 - RNA, 120 nt, Artificial Sequence
  Description of Artificial Sequence Synthetic polynucleotide
cuaaaucacccauucaguacaucgauaucgguaauuauacaguuuccuguuuaccuuuuacaauuaauugccaggaaccuaaauuggguagucuuguagugcguuguucguucuaugaag

SEQ ID NO: 17 - DNA, 28 bp, Artificial Sequence
  Description of Artificial Sequence Synthetic primer
taacaaacatgctgattttgacacatgg

SEQ ID NO: 18 - DNA, 20 bp, Artificial Sequence
  Description of Artificial Sequence Synthetic primer
ccaggcacgacaaaacccac

SEQ ID NO: 19 - DNA, 30 bp, Artificial Sequence; identical to SEQ ID NO: 3
  Description of Artificial Sequence Synthetic primer

SEQ ID NO: 20 - DNA, 34 bp, Artificial Sequence; identical to SEQ ID NO: 4
  Description of Artificial Sequence Synthetic primer

SEQ ID NO: 21 - DNA, 23 bp, Artificial Sequence
  Description of Artificial Sequence Synthetic primer
gaattgtgcgtggatgaggctgg

SEQ ID NO: 22 - RNA, 20 nt, Artificial Sequence
  Description of Artificial Sequence Synthetic oligonucleotide
ucagaacagcaacauuugaa

SEQ ID NO: 23 - DNA, 48 bp, Artificial Sequence
  Description of Artificial Sequence Synthetic oligonucleotide
cgggagggtgggcgggagggtgggcgggagggtgggcgggagggtggg

SEQ ID NO: 24 - DNA, 48 bp, Artificial Sequence
  Description of Artificial Sequence Synthetic oligonucleotide
cccaccctcccgcccaccctcccgcccaccctcccgcccaccctcccg

SEQ ID NO: 25 - DNA, 45 bp, Artificial Sequence
  Description of Artificial Sequence Synthetic oligonucleotide
ggagggtggggattaattgtaaaaggtgaacgggcgggagggtgg

SEQ ID NO: 26 - DNA, 45 bp, Artificial Sequence
  Description of Artificial Sequence Synthetic oligonucleotide
ccaccctcccgcccgttcaccttttacaattaatccccaccctcc

SEQ ID NO: 27 - DNA, 45 bp, Artificial Sequence
  Description of Artificial Sequence Synthetic oligonucleotide
ggagggtggggattaattgtaaaaggtaaacgggcgggagggtgg

SEQ ID NO: 28 - DNA, 45 bp, Artificial Sequence
  Description of Artificial Sequence Synthetic oligonucleotide
ccaccctcccgcccgtttaccttttacaattaatccccaccctcc

SEQ ID NO: 29 - DNA, 25 bp, Artificial Sequence
  Description of Artificial Sequence Synthetic primer
gtcactcgtgacatagcatctacag

SEQ ID NO: 30 - DNA, 22 bp, Artificial Sequence
  Description of Artificial Sequence Synthetic primer
ttgtgcgtaatatcgtgccagg

SEQ ID NO: 31 - DNA, 22 bp, Artificial Sequence
  Description of Artificial Sequence Synthetic primer
ttgtgcgtaatatcgtgccagg

SEQ ID NO: 32 - DNA, 120 bp, Artificial Sequence
  Description of Artificial Sequence Synthetic polynucleotide
taacaaacatgctgattttgacacatggtttagccagcgtggtggtagttatactaatgacaaagcttgcccattgattgctgcagtcataacaagagaagtgggttttgtcgtgcctgg

SEQ ID NO: 33 - DNA, 122 bp, Artificial Sequence
  Description of Artificial Sequence Synthetic polynucleotide
taatacgactcactatagggacacatggtttagtcagcggttttagagctagaaatagcaagttaaaataaggctagtccgttatcaacttgaaaaagtggcaccgagtcggtgcttttttt

SEQ ID NO: 34 - DNA, 122 bp, Artificial Sequence
  Description of Artificial Sequence Synthetic polynucleotide
taatacgactcactatagggacacatggtttagccagcggttttagagctagaaatagcaagttaaaataaggctagtccgttatcaacttgaaaaagtggcaccgagtcggtgcttttttt

SEQ ID NO: 35 - DNA, 19 bp, Artificial Sequence
  Description of Artificial Sequence Synthetic primer
taatacgactcactatagg

SEQ ID NO: 36 - DNA, 25 bp, Artificial Sequence
  Description of Artificial Sequence Synthetic primer
aaaaaaagcaccgactcggtgccac

SEQ ID NO: 37 - DNA, 22 bp, Artificial Sequence
  Description of Artificial Sequence Synthetic primer
caacacgaacgtcatgatactc

SEQ ID NO: 38 - DNA, 120 bp, Artificial Sequence
  Description of Artificial Sequence Synthetic polynucleotide
ctaaatcacccattcagtacatcgatatcggtaattatacagtttcctgtttaccttttacaattaattgccaggaacctaaattgggtagtcttgtagtgcgttgttcgttctatgaag

SEQ ID NO: 39 - DNA, 34 bp, Artificial Sequence; identical to SEQ ID NO: 4
  Description of Artificial Sequence Synthetic primer

SEQ ID NO: 40 - DNA, 122 bp, Artificial Sequence
  Description of Artificial Sequence Synthetic polynucleotide
taatacgactcactataggattaattgtaaaaggtgaacgttttagagctagaaatagcaagttaaaataaggctagtccgttatcaacttgaaaaagtggcaccgagtcggtgcttttttt

SEQ ID NO: 41 - DNA, 122 bp, Artificial Sequence
  Description of Artificial Sequence Synthetic polynucleotide
taatacgactcactataggattaattgtaaaaggtaaacgttttagagctagaaatagcaagttaaaataaggctagtccgttatcaacttgaaaaagtggcaccgagtcggtgcttttttt

SEQ ID NO: 42 - DNA, 47 bp, Artificial Sequence
  Description of Artificial Sequence Synthetic oligonucleotide
taatacgactcactataggtaacaaacatgctgattttgacacatgg

SEQ ID NO: 43 - DNA, 20 bp, Artificial Sequence
  Description of Artificial Sequence Synthetic primer
atatcgccgcgctcgtcgtc

SEQ ID NO: 44 - DNA, 21 bp, Artificial Sequence
  Description of Artificial Sequence Synthetic primer
atgtcgtcccagttggtgacg

SEQ ID NO: 45 - DNA, 21 bp, Artificial Sequence
  Description of Artificial Sequence Synthetic primer
atgtcgtcccagttggtgacg

SEQ ID NO: 46 - DNA, 20 bp, Artificial Sequence
  Description of Artificial Sequence Synthetic primer
atatcgccgcgctcgtcgtc

SEQ ID NO: 47 - DNA, 20 bp, Artificial Sequence
  Description of Artificial Sequence Synthetic primer
atatcgccgcgctcgtcgtc

SEQ ID NO: 48 - DNA, 21 bp, Artificial Sequence
  Description of Artificial Sequence Synthetic primer
atgtcgtcccagttggtgacg

SEQ ID NO: 49 - DNA, 44 bp, Artificial Sequence
  Description of Artificial Sequence Synthetic oligonucleotide
taatacgactcactatagggtcactcgtgacatagcatctacag

SEQ ID NO: 50 - DNA, 42 bp, Artificial Sequence
  Description of Artificial Sequence Synthetic oligonucleotide
taatacgactcactatagggaattgtgcgtggatgaggctgg

SEQ ID NO: 51 - RNA, 20 nt, Artificial Sequence
  Description of Artificial Sequence Synthetic oligonucleotide
cucaccgugcccagugagcg

SEQ ID NO: 52 - RNA, 20 nt, Artificial Sequence
  Description of Artificial Sequence Synthetic oligonucleotide
aauucgagcagcugaaacug

SEQ ID NO: 53 - RNA, 20 nt, Artificial Sequence
  Description of Artificial Sequence Synthetic oligonucleotide
uugaacagcagcuguguaug

SEQ ID NO: 54 - RNA, 20 nt, Artificial Sequence
  Description of Artificial Sequence Synthetic oligonucleotide
cacuggcugucgcuucucaa

SEQ ID NO: 55 - DNA, 24 bp, Artificial Sequence
  Description of Artificial Sequence Synthetic oligonucleotide
ttagatgaaatctaaaacaacacg

SEQ ID NO: 56 - DNA, 176 bp, Artificial Sequence
  Description of Artificial Sequence Synthetic SARS-CoV-2 PCR template containing a cytosine SNP at position 28144
gaattgtgcgtggatgaggctggttctaaatcacccattcagtacatcgatatcggtaattatacagtttcctgtttcaccttttacaattaattgccaggaacctaaattgggtagtcttgtagtgcgttgttcgttctatgaagactttttagagtatcatgacgttcgtgttg

SEQ ID NO: 57 - DNA, 23 bp, Artificial Sequence
  Description of Artificial Sequence Forward primer for Influenza A amplification from synthetic DNA
gcattytggacaaavcgtctacg

SEQ ID NO: 58 - DNA, 23 bp, Artificial Sequence
  Description of Artificial Sequence Reverse primer for Influenza A amplification from synthetic DNA
gcattytggacaaavcgtctacg

SEQ ID NO: 59 - DNA, 160 bp, Artificial Sequence
  Description of Artificial Sequence Synthetic PCR template to generate Influenza A DNA amplicons
gctctcatggaatggctaaagacaagaccaatcttgtcacctctgactaaggggattttaggatttgtgttcacgctcaccgtgcccagtgagcgaggactgcagcgtagacgctttgtccaaaatgccctaaatgggaatggggacccgaacaacatgg

SEQ ID NO: 60 - DNA, 23 bp, Artificial Sequence
  Description of Artificial Sequence Forward primer for Influenza B amplification from synthetic DNA
tcctcaaytcactcttcgaggcg

SEQ ID NO: 61 - DNA, 21 bp, Artificial Sequence
  Description of Artificial Sequence Reverse primer for Influenza B amplification from synthetic DNA
cggtgctcttgaccaaattgg

SEQ ID NO: 62 - DNA, 160 bp, Artificial Sequence
  Description of Artificial Sequence Synthetic PCR template to generate Influenza B DNA amplicons
tacagtggaggatgaagaagatggccatcggatcctcaattcactcttcgagcgtcttaatgaaggacattcaaagccaattcgagcagctgaaactgcggtgggagtcttatcccaatttggtcaagagcaccgactatcaccagaagagggagacaat

SEQ ID NO: 63 - DNA, 24 bp, Artificial Sequence
  Description of Artificial Sequence Forward primer for RSV amplification from synthetic DNA
ggcaaatatggaaacatacgtgaa

SEQ ID NO: 64 - DNA, 20 bp, Artificial Sequence
  Description of Artificial Sequence Reverse primer for RSV amplification from synthetic DNA
catgggcacccatattgtaa

SEQ ID NO: 65 - DNA, 130 bp, Artificial Sequence
  Description of Artificial Sequence Synthetic PCR template to generate RSV DNA amplicons
ggcaaatatggaaacatacgtgaacaagcttcacgaaggctccacatacacagctgctgttcaatacaatgtcctagaaaaagacgatgaccctgcatcacttacaatatgggtgcccatgttccaatca

SEQ ID NO: 66 - DNA, 32 bp, Artificial Sequence
  Description of Artificial Sequence FAM fluorescent probe with Iowa Black quencher that recognizes SARS- CoV-2 with a thymine at position 28144
tcctgtttaccttttacaattaattgccagga

SEQ ID NO: 67 - DNA, 32 bp, Artificial Sequence
  Description of Artificial Sequence FAM fluorescent probe with Iowa Black quencher that recognizes SARS- CoV-2 with a cytosine at position 28144
tcctgttcaccttttacaattaattgccagga

SEQ ID NO: 68 - DNA, 32 bp, Artificial Sequence
  Description of Artificial Sequence Texas Red fluorescent probe with Iowa Black quencher that recognizes Influenza A sequence
cagtcctcgctcactgggcacggtgagcgtga

SEQ ID NO: 69 - DNA, 32 bp, Artificial Sequence
  Description of Artificial Sequence Yakima Yellow fluorescent probe with Iowa Black quencher that recognizes Influenza B sequence
tcccaccgcagtttcagctgctcgaattggct

SEQ ID NO: 70 - DNA, 32 bp, Artificial Sequence
  Description of Artificial Sequence Cy3 fluorescent probe with Iowa Black quencher that recognizes RSV sequence
gctccacatacacagctgctgttcaatacaat

SEQ ID NO: 71 - DNA, 32 bp, Artificial Sequence; identical to SEQ ID NO: 70
  Description of Artificial Sequence TAMRA fluorescent probe with Iowa Black quencher that recognizes RSV sequence

(19) United States
(12) Patent Application Publication
(10) Pub. No.: US 2023/0250495 A1
(43) Pub. Date: Aug. 10, 2023

(54) CAS9-BASED DIAGNOSTIC ASSAY AND METHODS OF USING

(71) Applicants: REGENTS OF THE UNIVERSITY OF MINNESOTA, Minneapolis, MA (US); PRESIDENT AND FELLOWS OF HARVARD COLLEGE, Cambridge, MA (US)

(72) Inventors: Mark Osborn, Minneapolis, MN (US); Gregory A. Newby, Cambridge, MA (US)

(21) Appl. No.: 17/922,633

(22) PCT Filed: May 1, 2021
(86) PCT No.: PCT/US2021/030370

§ 371 Date: Nov. 1, 2022

Related U.S. Application Data
(60) Provisional application No. 63/018,933, filed on May 1, 2020.

(51) Int. Cl. C12Q 1/70; C12Q 1/6844

(52) U.S. Cl. CPC C12Q 1/701; C12Q 1/6844; C12Q 1/6818

(57) ABSTRACT

This disclosure describes a rapid, high throughput, facile testing platform. Amplified DNA and CRISPR/Cas9-bound products are analyzed via a lateral flow assay (LFA), and the assay does not require specialized infrastructure. In some embodiments, the testing platform may be used to detect SARS-CoV-2, including, for example, as a test for COVID-19.

### CONTINUING APPLICATION DATA

This application claims the benefit of U.S. Provisional Application Ser. No. 63/018,933, filed May 1, 2020, which is incorporated by reference herein.

### SEQUENCE LISTING

The instant application contains a Sequence Listing which has been submitted electronically in ASCII format and is hereby incorporated by reference in its entirety. Said ASCII copy, created on Apr. 20, 2023, is named 0110_000653WO01_SL.txt and is 18.8 kilobytes in size.

### BACKGROUND

The coronavirus disease 2019 (COVID-19) is caused by a novel coronavirus named “Severe Acute Respiratory Syndrome CoronaVirus 2” (SARS-CoV-2). As of the end of April 2021, the COVID-19 pandemic has infected more than 32 million in the US, caused more than 575,000 deaths, and resulted in massive community disruptions across the globe.

At the time of the invention (April 2020), COVID-19 testing required labor-intensive RNA isolation, specialized instrumentation for real time RT-PCR, and results were taking more than 24 hours to obtain. Thus, a critical need existed for a rapid, high throughput, easy-to-perform COVID-19 point-of-care test.

### SUMMARY OF THE INVENTION

This disclosure describes a rapid, high throughput, facile testing platform. Amplified DNA and CRISPR/Cas9-bound products may be analyzed via a lateral flow assay (LFA), the technology commonly used for rapid testing, such as home pregnancy tests, which does not require specialized infrastructure. In some embodiments, the testing platform may be used to detect SARS-CoV-2, including, for example, as a test for COVID-19.

In one aspect, this disclosure describes a method that includes amplifying a nucleotide to form a target polynucleotide. The amplification includes using a first primer and a second primer, and the first primer includes a label. The method further includes exposing the target nucleotide to a Cas protein from a clustered regularly interspaced palindromic repeat (CRISPR) system such as Streptococcus pyogenes Cas9 and a gRNA, wherein the Cas9 includes a label, to form a target nucleotide-Cas9 complex. Finally, the method includes detecting the target nucleotide-Cas9 complex including, for example, in a lateral flow assay, a fluorometric assay, or a colorimetric assay.

In some embodiments, the method includes detecting SARS-CoV-2.

In another aspect, this disclosure describes a composition including a target nucleotide, a labeled Cas9 protein (e.g., Cas9), and a gRNA. The target nucleotide includes a label. Exemplary labels for the target nucleotide include FITC, a fluorescein amidite (FAM), or digoxigenin (DIG). An exemplary label for the Cas9 includes biotin.

In some embodiments, the target nucleotide includes a SARS-CoV-2-specific sequence.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

Herein, “up to” a number (for example, up to 50) includes the number (for example, 50).

The term “in the range” or “within a range” (and similar statements) includes the endpoints of the stated range.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

As used herein “nucleotide” and/or grammatical equivalents thereof can refer to a single nucleotide, a single nucleotide analog, any number of linked nucleotides, any number of linked nucleotide analogs, or combinations thereof. For example, “nucleotide” can refer to a single nucleotide or a strand of linked nucleotides that can also be referred to as a polynucleotide.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1A shows a schematic of the recombinase polymerase amplification (RPA) process. FIG. 1B shows a schematic of the test strip of the PCRD nucleic acid lateral flow immunoassay (Abingdon Health, York, United Kingdom). FIG. 1C shows a schematic of the test strip of the HybriDetect-Universal Lateral Flow Assay Kit (Milenia Hybritech, Gießen, Germany).

FIG. 2A-FIG. 2B show schematics of DNA and RNA synthetic fragments (Ultramers) and primers used for amplifying COVID-19 sequences from experimental or patient samples. FIG. 2C shows the results of a lateral flow assay (LFA) performed as described in Example 1.

FIG. 3A-FIG. 3B show the result of RT-PCR of SARS-CoV-2 RNA Ultramers for sequences from ORF8 of SARS-Co-V2 at the indicated dilutions (2 μM RNA template was input in to RT reaction), performed as described in Example 2.

FIG. 4A shows dilutions of primers run on an agarose gel, as further described in Example 3. FIG. 4B shows LFA results with PCR products, performed as described in Example 3.

FIG. 5 shows LFA results with PCR products, performed as described in Example 4.

FIG. 6 shows LFA results with PCR products, performed as described in Example 5.

FIG. 7 shows LFA results with PCR products, performed as described in Example 6.

FIG. 8 shows LFA results with PCR products, performed as described in Example 7.

FIG. 9 shows LFA results with RPA products, performed as described in Example 8.

FIG. 10 shows LFA results with PCR products, performed as described in Example 9.

FIG. 11A to 11E show results from Example 12. Recombinase polymerase amplification (RPA) and nucleic acid detection by lateral flow assay. (FIG. 11A) Recombinase polymerase amplification. Recombinase proteins complex with primers and facilitate strand displacement and binding to homologous target sequence(s) without requiring temperature alterations (i.e., cycling). The DNA polymerase initiates synthesis from the primers and amplifies target DNA exponentially. (FIGS. 11B to 11E) Analyte detection by lateral flow assay (LFA). (FIG. 11B) Reagent components for detection via LFA (Au-NP=gold nanoparticle, Ab=antibody, FITC=fluorescein isothiocyanate). (FIG. 11C) Embedded in the flow assay device conjugate pad are gold nanoparticles decorated with rabbit anti-FITC antibodies. The test and assay control bands are coated with a biotin ligand or anti-rabbit antibodies, respectively. A red arrow indicates the direction of sample flow through the conjugation pad. In the absence of a molecule that is labeled by both FITC and biotin, the Au-NPs flow to and accumulate at the assay control band, where they are bound by anti-rabbit antibodies. Dual FITC:biotin-labeled substrates are bound first by the anti-FITC Au-NPs and then accumulate at the test band via capture of the biotin label by its ligand. Because Au-NPs are in excess, Au-NPs containing only the rabbit-anti-FITC Ab also flow to the assay control band. Accumulated Au-NPs are observed, and results are interpreted as (FIG. 11D) negative when only an assay control band is present or (FIG. 11E) positive with both test and assay control bands are visible.

FIG. 12A to 12G show results from Example 12. Biotinylated ‘dead’ Cas9 (bdCas9) and PAM-rich competitor soak DNA allows for the detection of target nucleic acids. (FIG. 12A) SARS-CoV-2 genome and detection strategy. The viral genes are shown as well as the D614G and N501Y amino acid mutations in the S gene and the L84S alteration in ORF8a. The locations of the WHO and CDC genes analyzed in their respective diagnostic assays are indicated with red arrows. The sequence in the ORF8a gene targeted for CRISPR/Cas9 sgRNA detection in the present study is shown with the L84S polymorphic nucleotide at position 28144 of SARS-Co-V2 in the ORF8 gene (indicated in red) and the CRISPR/Cas9 protospacer adjacent motif (highlighted in green). (FIG. 12B, FIG. 12C) Dual-labeled PCR primer strategy results in false positives. Amplification was performed with a FITC-labeled forward and biotinylated reverse primer in the absence (−) or presence (+) of a template, and products were analyzed via LFA (FIG. 12C). ‘Blank’ refers to an LFA test strip loaded with no amplicon. (FIG. 12D, FIG. 12E) Uncoupling the FITC and biotin labeling of DNA using a FITC primer and bdCas9 allows for nucleic acid detection. Amplification was performed with a FITC-labeled forward and unlabeled reverse primer. The amplicon was then interrogated with a SARS-CoV-2-specific or mismatched control sgRNA. (FIG. 12E) The presence of test bands in samples with either sgRNA demonstrates non-specific dCas9 binding of target DNA. (FIG. 12F, FIG. 12G) A competing PAM-rich soak double-stranded oligonucleotide prevents non-specific bdCas9/mismatched sgRNA binding. (FIG. 12F) A double-stranded oligonucleotide rich in GGG trinucleotide PAMs was included in the reaction mixture containing FITC-labeled COVID DNA amplicons and a COVID sgRNA or an unmatched control sgRNA. (FIG. 12G) LFA of COVID-19 amplicons with COVID-19 or control/mismatched sgRNA LFA detection shows specificity in the presence of the competitor soak DNA. Blue and black arrows in (FIG. 12C), (FIG. 12E), and (FIG. 12G) designate the assay control and test bands on the LFA, respectively. Results are representative of at least four independent experiments.

FIG. 13A to 13C show results from Example 12. Tuning primer concentrations for detection of LFA signals. (FIG. 13A) PCR primers labeled with FITC (forward) or biotin (reverse) were designed for amplification of SARS-CoV-2 DNA. (FIG. 13B) Amplification was performed with the indicated concentrations of primer in the absence or presence of template and analyzed by LFA. (FIG. 13C) Agarose gel analysis of primer concentrations employed in (FIG. 13B).

FIGS. 14A to 14C show results from Example 12. Nucleic acid sequences for sgRNAs and soak DNA. (FIG. 14A) The SARS-Co-V2 sgRNA sequences are shown (5′-3′) with the L84S SNP target bases corresponding to SARS-CoV-2 nucleotide position 28144 (shown in red). (FIG. 14B) Soak DNA sequences. The PAM-rich soak DNA is rich in GGG trinucleotide PAM sequences. The ORF8a S84 C and L84 T SNP soak sequences are shown with the sgRNA binding site underlined and the respective SNP indicated in red. (FIG. 14C). Irrelevant DNA is not bound by COVID gRNA. An irrelevant DNA labeled with FITC was incubated with a perfectly matched sgRNA or the COVID-19 sgRNA.

FIGS. 15A and 15B show results from Example 12. Optimization of rapid SARS-Co-V2 nucleic acid detection. (FIG. 15A) Simultaneous RPA and bdCas9 detection results in false positives. Room temperature RPA of a SARS-CoV-2 template was performed in the presence of bdCas9 plus 10 or 20 μM competitor (i.e., PAM) soak DNA with a COVID-19 or mismatched sgRNA and analyzed by LFA. (FIG. 15B) Sequential RPA and Cas9 detection. RPA was performed at room temperature followed by amplicon incubation with bdCas9. RPA products were incubated with the indicated concentrations of competitor PAM soak DNA for 20, 40, or 60 min with either a COVID-19 (C19) or mismatched (MM) irrelevant sgRNA, followed by LFA. Blue arrows show the assay control band and black arrows indicate a test band. Images are representative of three independent experiments. LFA-labeled RPA in FIG. 15A represents a no DNA template control.

FIGS. 16A and 16B show results from Example 12. Correlative genome analysis of SARS-Co-V2 variants. (FIG. 16A) The correlation between the D614G (nucleotide 1842) strain of SARS-Co-V2 and strain with L84S due to a single-nucleotide polymorphism at SARS Co-V2 genome nucleotide coordinate 28114 was assessed for the Midwestern state of Minnesota (USA). A pie graph for 1610 patients shows the relationship between the amino acid aspartic acid (D) or glycine (G) at amino acid position 614 and cytosine (C) or thymine (T) nucleotide at 28114. (FIG. 16B) The relationship between the ORF8a L84 or S84 and N501Y is shown. The genomes were analyzed from 1816 patients in the United Kingdom.

FIGS. 17A and 17B show results from Example 12. Fluorescence-based CRISPR/Cas9 nuclease detection of SARS-CoV-2 DNA. (FIG. 17A) Design of a CRISPR/Cas9 nuclease fluorescence detection assay. SARS-CoV-2 DNA amplicons were mixed with DNA probes containing a 5′ fluorescein fluorophore and 3′ Iowa black quencher. The probe and amplicons were denatured and renatured, resulting in a heteroduplex of amplicon:probe DNA that was incubated with CRISPR/Cas9 nuclease complexed with COVID-19, or control sgRNAs. Cas9 nucleolytic activity uncouples FAM from the quencher, resulting in a fluorescent signal that can be measured with a fluorometer. (FIG. 17B) CRISPR/Cas9 nuclease fluorescent signal detection of SARS-CoV-2 DNA. A time course was performed to evaluate fluorescence signal intensity generated by Cas9 nuclease conjugated with a COVID-19 sgRNA, two separate unmatched control sgRNAs, or hybridized PCR:probe duplexes (DNA/probe) alone. Shown is the mean relative fluorescent value (RFU) and standard deviation from four independent experiments. p-value (one-way analysis of variance (ANOVA) and Tukey's multiple comparisons test) of ****<0.0001 is shown with asterisks.

FIGS. 18A to 18C show results from Example 12. Multiplex CRISPR/Cas9 fluorescence detection of viral respiratory pathogens. (FIG. 18A) CRISPR/Cas9 shows high specificity in distinguishing respiratory pathogen targets. Individual DNA amplicons and fluorescent probes for SARS-CoV-2 (FAM-labeled), influenza A (Flu A; TxRed®-labeled) or B (Flu B; Yakima Yellow®-labeled), or respiratory syncytial virus (RSV; TAMRA-labeled) were annealed and interrogated with Cas9 and sgRNAs for each virus DNA. The DNA target amplicons are indicated at the top of each graph and the sgRNAs are labeled vertically on the y-axis. The heat maps represent the mean fluorescent values from three independent experiments performed in duplicate. The horizontal color bars at the top of each heat map are the values normalized to the highest fluorescent signal obtained. (FIG. 18B) Fluorescence values are plotted as the mean of the three independent experiments performed in duplicate from (FIG. 18A). Statistical evaluation of these data was done using one-way ANOVA and Tukey's multiple comparison to show fluorescence signal above other analytes. *, ***, *** indicates p=<0.05, p=<0.001, and p=<0.0001 respectively, for the appropriate matched sgRNA versus the highest corresponding fluorescent value for an unmatched sgRNA. The y-axis is relative fluorescent units (RFU), the x-axis represents the DNA target, and the label at the top represents the sgRNA for COVID, Influenza A or B, RSV, and template:probe hybrids alone (ODN). (FIG. 18C) Multiplex detection of four respiratory viral pathogen sequences. Annealed probe:DNA complexes for each target were pooled and interrogated simultaneously with all four sgRNAs complexed with Cas9. Background subtracted fluorescent units (BFU) obtained by subtracting the fluorescence of controls (annealed DNA/Probe without Cas9) are shown as the mean and standard deviation of three experiments. The color-coded bars in the graph correspond to the color-coded identifiers at the left. The fluorescent values were acquired using a real-time PCR instrument that measured fluorescence signal every 30 s (1 cycle=30 s for 121 cycles). The x-axes for the graphs in (FIG. 18B) and (FIG. 18C) are labeled as ‘cycles’ which represents the time at which fluorescence images were captured during the one hour 37° C. isothermal assay (1 cycle=30 s).

FIGS. 19A to 19D show results from Example 12. SARS-CoV-2 genomic RNA detection by LFA and fluorescence. (FIG. 19A) Viral RNA was diluted serially from 1:10 to 1:10,000 and reverse transcribed and analyzed by qRT-PCR using the CDC N1 and N2 primer:probe sets. Shown are triplicate samples of the mean±standard deviation (SD) cycle threshold (Ct). (FIG. 19B) Viral cDNA was amplified as in (FIG. 19A) using ORF8a primers and analyzed by agarose gel electrophoresis. Mw=molecular weight and NTC=no template control. (FIG. 19C) Fluorescence detection of SARS-CoV-2 DNA targets. PCR products from serially diluted viral cDNA was hybridized with a COVID-19 probe and incubated with a Cas9:control sgRNA or COVID sgRNA and analyzed for fluorescence generation under isothermal (37° C.) conditions for one hour. Heat maps are shown of the mean of four experiments with the dilution series indicated at the top. Raw fluorescence value scales are shown at the top and the y-axis is the sample identification of DNA:probe alone (ODN) and DNA:probe hybrids interrogated with a control or COVID sgRNA. The x-axis represents the thirty second time points at which fluorescence was measured over the course of one hour (121 total measurements). (FIG. 19D) Copy number limit of detection (LOD). DNA obtained via PCR of SARS-CoV-2 reverse transcribed RNA was quantified and serially diluted to test the limit of detection using Cas9 nuclease generated fluorescence products. DNA:probe without Cas9 fluorescence values were subtracted and the y-axis represents these values as background subtracted fluorescence units (BFU). Values were measured every thirty seconds as in (FIG. 19C). Data are the mean values from three experiments. (FIG. 19E) Serially diluted ORF8a amplicons from viral cDNA were analyzed by LFA.

FIGS. 20A and 20B show results from Example 12. Quantitative reverse transcriptase PCR of coronaviral genomic RNA. RNA from the USA-WA1/2020 strain was diluted from 1:10 to 1:10,000, reverse transcribed, and analyzed using the CDCN gene primer:probe sets. Data are duplicates representative of three analyses and (FIG. 20A) is the N1 probe and (FIG. 20B) is the N2 probe. The y-axis shows Rn that is the reporter fluorescent signal normalized to ROX and the x-axis shows cycle number.

FIG. 21 shows results from Example 12. Optimization of LFA for SARS-Co-V2 genomic RNA. DNA amplicons from reverse transcribed RNA, and analyzed using the Centers for Disease Control and Prevention (CDC) N gene primer:probe sets. Data are duplicates representative of three analyses and (A) is the N1 probe and (B) is the N2 probe. The y-axis shows (cycle threshold) Rn that is the reporter fluorescent signal normalized to ROX and the x-axis shows cycle number.

FIGS. 22A to 22E show results from Example 12. Single-nucleotide resolution of a SARS-Co-V2 variant. (FIG. 22A, FIG. 22B) Comparison of wild-type Cas9 (FIG. 22A) and SpyFi™ Cas9 (FIG. 22B) for single-nucleotide recognition COVID-19 DNA amplicons with a thymine at position 28114 were interrogated with a sgRNA either perfectly matched (T sgRNA) or mismatched by a single base pair (C sgRNA). The fluorescence values for three independent experiments for (FIG. 22A) wild-type Cas9 and (FIG. 22B) SpyFi™ Cas9 are shown as mean and standard deviation. Controls were an unmatched sgRNA with the indicated Cas9 and probe:DNA hybridization products with no addition of Cas9. Three independent experiments in duplicate were performed with both Cas9 and SpyFi™ included on the same 96-well assay plate. (FIG. 22C-FIG. 22E) Single nucleotide detection via LFA. (FIG. 22C) Experimental schema. SARS-Co-V2 DNA amplicons with a thymine (T SNP) or cytosine (C SNP) at ORF8a position 28114 were amplified using a FITC-labeled primer. bdCas9 was complexed with a perfectly matched or single base pair mismatched sgRNA. Decoy soak DNA was included as a PAM-rich soak or was mismatched by a single base pair to the specific sgRNA complexed with bdCas9. Dashed lines indicate the blockade of Cas9 by soak DNA. COVID target DNA with a thymine (FIG. 22D) or cytosine (FIG. 22E) were interrogated with the sgRNAs shown under the LFA test strips. Soak refers to whether the reaction contained a decoy DNA with a cytosine (FIG. 22D), thymine (FIG. 22E), or the PAM-rich soak DNA. Data are representative of five independent experiments and the blue and black arrows represent assay control and test bands, respectively.

### DETAILED DESCRIPTION OF ILLUSTRATIVE EMBODIMENTS

This disclosure describes a rapid, high throughput, facile testing platform for user defined sequence(s) of interest including but not limited to viral, bacterial, plantae, or mammalian nucleic acids.

In one aspect, amplified DNA and CRISPR/Cas9-bound products are analyzed via a lateral flow assay (LFA), the technology commonly used for rapid testing, such as home pregnancy tests, which does not require specialized infrastructure. In some embodiments, the testing platform may be used to detect SARS-CoV-2 including, for example, as a test for COVID-19.

In another aspect, amplified DNA (e.g. SARS-Co-V2, influenza, respiratory syncytial virus etc) and CRISPR/Cas9-bound products may be analyzed by the detection of a detectable signal such as fluorescence or a colorimetric value.

### SARS-CoV-2

SARS-CoV-2, the coronavirus that causes COVID-19, has a genome that varies from 29.8 kb to 29.9 kb. The genome includes ORF1ab (encoding orf1ab polyproteins), which makes up more than two-third of the genome; genes encoding structural proteins including surface (S), envelope (E), membrane (M), and nucleocapsid N proteins; and 6 accessory proteins, encoded by the ORF3a, ORF6, ORF7a, ORF7b, and ORF8 genes. (Khailanya et al. Gene Reports 2020: 100682.)

### SARS-CoV-2 Type I and Type II

SARS-CoV-2 may be classified into two major genotypes, Type I and Type II. (See, e.g., Wang et al., https://www.medrxiv.org/content/10.1101/2020.02.25.20027953v2). These types suggest two, possibly three, major outbreak sources. Although both types are found in humans, Type II is believed to be more contagious (transmissible) than Type I.

### Primer Dimers May Cause False Positives

As shown in Examples 1-3, amplification with primers labeled with DIG/FAM/FITC paired with a biotinylated primer can cause a positive test band in the absence of template RNA or DNA. This false positive test band was due to free primers or primer dimers, consistent with a previous report in the literature (see Li et al., Analyst, 2019; 144:31-67).

Others have alleviated false positives caused by free primers or primer dimers using specialized chemistry (Hoshika et al. Nucleic Acids Symp. Ser., 2008; 129-130) or by using of biotin conjugated nanoparticles that require specialized synthesis of the nanoparticles and the Cas9 associated sgRNA (Wang et al, https://www.biorxiv.org/content/10.1101/702209v1.full).

### Biotinylated Cas9-Based Lateral Flow Assay Using an Unlabeled Primer

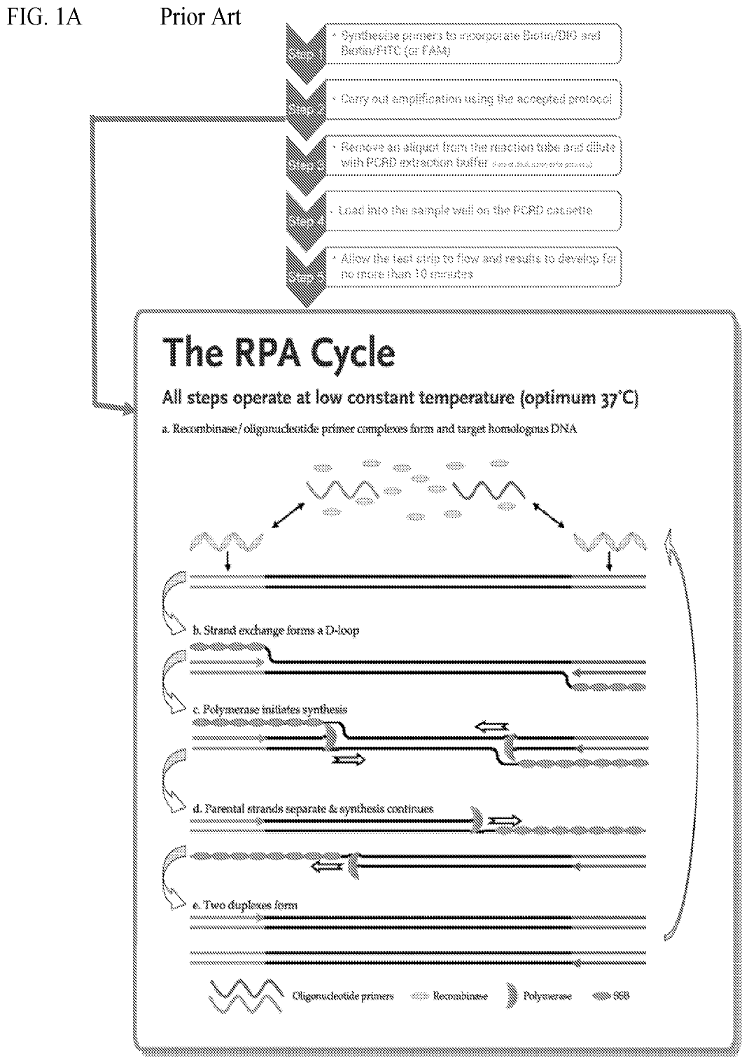

It was hypothesized that the amplification and detection steps of LFA could be uncoupled by using one labeled primer (e.g., with FITC) and one unlabeled primer and that this uncoupling might decrease or eliminate the false positives associated with free primers or primer dimers. The resulting amplification product would not yield a detectable band because, in the absence of biotin, the LFA strip would not allow for binding/visualization to the amplification product (see FIG. 1C). It was further hypothesized that detection of the amplification product (or other analyte) could be restored by employing a biotinylated version of Cas9 (for example, the biotinylated Cas9 available from Sigma Aldrich, www.sigmaaldrich.com/catalog/product/sigma/dcas9prot?lang=en&region=US) and a guide RNA (gRNA) (or more specifically a single guide RNA (sgRNA)) specific for the amplification product (or other analyte).

As further described in Example 4-9, amplification (by PCR or RPA) and detection (by LFA) may be uncoupled by using an unlabeled primer. Moreover, detection may be restored by providing a biotinylated version of Cas9 and a guide RNA (gRNA) specific for the amplification product. Without wishing to be bound by theory, it is believed that the biotinylated Cas9 binds the amplification product, amplified with DIG/FAM/FITC on one primer and no label on the other primer, mediated by the gRNA, resulting in a compound detectable by lateral flow assay (LFA) as the Cas9 biotin allows for visualization whilst the DIG or FAM is captured at the test band on the LFA strip.

Although Examples 4-9 describe the detection of SARS-CoV-2 and detection using LFA, the detection assay as described herein may be used for diagnostics, detection, and analysis of any desired amplification product or analyte, provided a suitable gRNA is provided to mediate binding of a labeled Cas9 to a labeled target nucleotide.

### Cas9-Based Diagnostic Assay

In one aspect, this disclosure describes a Cas-based diagnostic assay and compositions included in performing that assay. In some embodiments, the assay may be a lateral flow-based assay. In some embodiments, the assay may be a fluorometric- or colorimetric-based assay.

In some embodiments, the assay includes amplifying a target nucleotide to form a polynucleotide, exposing the nucleotide to labeled Cas9 and a gRNA to form a target nucleotide-Cas9 complex, and detecting the target nucleotide-Cas9 complex. In some embodiments, the target nucleotide-Cas9 complex may be detected in a lateral flow assay. In some embodiments, the target nucleotide-Cas9 complex may be detected in a fluorometric- or colorimetric-based assay.

The amplification of the nucleotides includes using a first primer and a second primer, wherein the first primer includes a label.

In some embodiments, the assay may include detecting SARS-CoV-2 including, for example, as a test for COVID-19.

In some embodiments, the assay may include detecting influenza or respiratory syncytial virus.

In other embodiments, the assay may include detecting user defined sequences from any particle or organism that is nucleic acid based.

As used herein, a label refers to a molecule that may be detected. Exemplary labels include fluorescent markers (such as fluorescein isothiocyanate (FITC) and a fluorescein amidite (FAM)), digoxigenin (DIG), biotin, a gold nanoparticle, colored latex, quantum dots, a ruthenium complex, a paramagnetic label, an enzyme label, a carbon nanoparticle, etc. (See Koczula et al. Essays Biochem, 2016; 60(1): 111-120.) Exemplary enzyme labels include alkaline phosphatase, peroxidase, and β-galactosidase.

In some embodiments, the label is preferably detectable in a lateral flow assay. In some embodiments, the labels may preferably include fluorescein isothiocyanate (FITC), a fluorescein amidite (FAM), digoxigenin (DIG), or biotin, or a combination thereof because of the ability of commercially available lateral flow assay strips to detect these labels.

In some embodiments, the label may be detectable in a fluorometric- or colorimetric-based assay. (See, e.g., Li et al., Trends in Biotechnology 2019; 37(7):730-743; Seamon et al. Anal. Chem. 2018; 90(11): 6913-6921; Chang et al., Microchimica Acta 2019; 186: 1-8.) In some embodiments, when the label is detectable in a fluorometric- or colorimetric-based assay, the label may include an enzyme label.

### Primers

As noted above, the first primer includes a label.

In some embodiments, the second primer is unlabeled. In some embodiments, the second primer is not labeled with biotin. In some embodiments, even if the second primer is labeled, the second primer is not labeled with a label that pairs with the label of the first primer for detection in a lateral flow assay.

The primers may include any suitable primers that amplify a nucleotide from any organism or nucleic acid to form a target nucleotide.

In some embodiments, the primers may be selected to amplify a portion of SARS-CoV-2. In some embodiments, the primers may be selected to amplify a nucleotide sequence from ORF1ab or ORF8.

In an exemplary embodiment, the primers may include taacaaacat gctgattttg acacatgg (SEQ ID NO: 1) (ORF1ab forward primer) and/or ccaggcacgacaaaacccac (SEQ ID NO: 2) (ORF1ab reverse primer).

In another exemplary embodiment, the primers may include ctaaatcacccattcagtacatcgatatcg (SEQ ID NO: 3) (ORF8 forward primer) and/or cttcatagaacgaacaacgcactacaagactacc (SEQ ID NO: 4) (ORF8 reverse primer).

In yet another exemplary embodiment, the primers may include gaattgtgcgtggatgaggctgg (SEQ ID NO: 5) (28112 forward primer) and/or caacacgaacgtcatgatactc (SEQ ID NO: 6) (28112 reverse primer).

In a further exemplary embodiment, the primers may include ctaaatcacccattcagtacatcgatatcg (SEQ ID NO: 3) (28112 forward primer) and/or caacacgaacgtcatgatactc (SEQ ID NO: 6) (28112 reverse primer).

### Cas

In some embodiments, the labeled Cas9 includes biotinylated Cas9. In an exemplary embodiment, the biotinylated Cas9 includes dCas9-3×FLAG-Biotin Protein (Sigma Aldrich, St. Louis, Mo.

The labeled Cas9 may include any suitable Cas9. For example, the Cas9 may include, a Cas9 from Neisseria meningitidis, Treponema denticola, Streptococcus thermophilus, Streptococcus pyogenes, Staphylococcus aureus, Francisella novicida, or Campylobacter jejuni, or a variant thereof, or a combination thereof. In some embodiments, the variant may preferably increase specificity; for example, SpyFi Cas9 (Aldevron, Fargo, N. Dak.). In an exemplary embodiment, the Cas9 includes Streptococcus pyogenes Cas9 (SpCas9). In another exemplary embodiment, the Cas9 includes a variant of Streptococcus pyogenes Cas9 (SpCas9) including, for example, SpyFi Cas9 (Aldevron, Fargo, N. Dak.).

In some embodiments, the Cas9 includes proteins from Cas12 or Cas13 or other CRISPR systems.

In some embodiments, an endonuclease such as a restriction enzyme may be used.
gRNA

The gRNA may be any suitable gRNA that enables a Cas protein such as Cas9 to form a complex with the target nucleotide sequence. The gRNA may, in some embodiments and under certain conditions, exhibit the ability to distinguish between target nucleotide sequences at single base resolution. Guide RNAs can also be referred to as single guide (sgRNA).

In addition to a sequence that allows binding to the target nucleotide sequence, the gRNA or sgRNA may include a “scaffold” sequence that allows Cas9 interaction and binding with a protospacer adjacent motif (PAM).

The PAM may be a PAM from Neisseria meningitidis, Treponema denticola, Streptococcus thermophilus, Streptococcus pyogenes, Staphylococcus aureus, Francisella novicida, or Campylobacter jejuni, or a Cas variant thereof, or a combination thereof including Cas12 and Cas13.

In some embodiments, the gRNA may be specific to a coronavirus. In some embodiments, the gRNA may include a SARS-CoV-2-specific sequence. In some embodiments, the gRNA may be specific to SARS-CoV-2. In some embodiments, the gRNA can include a sequence from ORF1ab of SARS-CoV-2. In some embodiments, the gRNA can include a sequence from ORF8 of SARS-CoV-2. In some embodiments, the gRNA can include a sequence from influenza. In some embodiments, the gRNA can include a sequence from respiratory syncytial virus. In some embodiments, the gRNA can include a sequence from Newcastle disease. In some embodiments, the gRNA can include a sequence from free nucleic acid or those found in organisms from bacteria, archaea, viruses, plantae, chordates such as reptiles, mammals etc.

In an exemplary embodiment, the gRNA may include AUUAAUUGUAAAAGGUGAAC (SEQ ID NO: 7) (COVID 28112C) or AUUAAUUGUAAAAGGUAAAC (SEQ ID NO: 8) (COVID 28112T).

In some embodiments, the gRNA may be selected such it specifically binds to one subtype of SARS-CoV-2 (for example, SARS-CoV-2 Type I or SARS-CoV-2 Type II as characterized by single nucleotide polymorphisms (SNPs) in the OPRF1ab gene at 28112 C>T).

In some embodiments, the gRNA may be selected such it specifically binds to a target nucleotide including a single nucleotide difference from another nucleotide. For example, the gRNA may be selected such it specifically binds to a particular SARS-CoV-2 variant (see https://nextstrain.org/ncov/global and the 1717 COVID genome entries in GenBank, available online at www.ncbi.nlm.nih.gov/genbank/sars-cov-2-seqs/).

In some embodiments, the gRNA may be specific to a respiratory syncytial virus (RSV). In some embodiments, the gRNA may be specific to an influenza RSV.

In some embodiments, the gRNA may be specific to an influenza virus. In some embodiments, the gRNA may include an Influenza A-specific sequence. In some embodiments, the gRNA may include an Influenza B-specific sequence. In some embodiments, the gRNA may be specific to Influenza A. In some embodiments, the gRNA may be specific to Influenza B. In some embodiments, the gRNA may be specific to a RSV amplicon.

In an exemplary embodiment, the sgRNA may include CUCACCGUGCCCAGUGAGCG (SEQ ID NO: 51) (Influenza A), AAUUCGAGCAGCUGAAACUG (SEQ ID NO: 52) (Influenza B), or UUGAACAGCAGCUGUGUAUG (SEQ ID NO: 53 (RSV).

### Amplifying a Nucleotide to Form a Target Nucleotide

The nucleotide to be amplified may include RNA, DNA, or cDNA.

The nucleotide may be amplified to form a target nucleotide by any suitable means including, for example, polymerase chain reaction (PCR) and recombinase polymerase amplification (RPA), loop-mediated isothermal amplification (LAMP) etc.

In some embodiments, including when amplification at a constant temperature is desired, RPA or LAMP may be preferred.

In some embodiments, the target nucleotide preferably includes a protospacer adjacent motif (PAM). In some embodiments, if the nucleotide to be amplified did not include a PAM (or does not include an effective PAM), a PAM may be introduced into the target nucleotide using specially designed primers for amplification of the nucleotide to form the target nucleotide.

The PAM may include a PAM from Neisseria meningitidis, Treponema denticola, Streptococcus thermophilus, Streptococcus pyogenes, Staphylococcus aureus, Francisella novicida, or Campylobacter jejuni, or a variant thereof, or a combination thereof. In some embodiments, the PAM in the target nucleotide preferably includes a PAM recognized by the Cas9 being used to form the target nucleotide-Cas9 complex. For example, when the Cas9 being used to form the target nucleotide-Cas9 complex includes SpCas9, the PAM in the target nucleotide may include 5′-NGG-3′, wherein N is any nucleobase. In an exemplary embodiment, the PAM may include 5′-AGG-3′.

In some embodiments, the target nucleotide may include a SARS-CoV-2-specific sequence. As used herein, a SARS-CoV-2-specific sequence includes a sequence that permits the detection of SARS-CoV-2 in a sample. For example, as described in the Examples, the target nucleotide may include a sequence from ORF1ab of SARS-CoV-2. In some embodiments, the target nucleotide may include a sequence from ORF8 of SARS-CoV-2.

In an exemplary embodiment, the target nucleotide includes ATTAATTGTAAAAGGTAAAC (SEQ ID NO: 9). In an additional exemplary embodiment, the target nucleotide includes ATTAATTGTAAAAGGTGAAC (SEQ ID NO: 10).

In another exemplary embodiment, the target nucleotide includes ATTAATTGTAAAAGGTAAACAGG (SEQ ID NO: 11). In a further exemplary embodiment, the target nucleotide includes ATTAATTGTAAAAGGTCAACAGG (SEQ ID NO: 12).

As described above, the target nucleotide is labeled as a result of amplification of a nucleotide with a labeled primer. In some embodiments, the resulting target nucleotide includes only a single label. Exemplary labels include fluorescent markers (such as fluorescein isothiocyanate (FITC) and a fluorescein amidite (FAM)), digoxigenin (DIG), biotin, a gold nanoparticle, colored latex, quantum dots, a ruthenium complex, a paramagnetic label, an enzyme label, a carbon nanoparticle, etc. (See Koczula et al. Essays Biochem, 2016; 60(1): 111-120.)

In some embodiments, the label is preferably detectable in a lateral flow assay. Exemplary labels detectable in a lateral flow assay include FITC, a fluorescein amidite (FAM), or digoxigenin (DIG) because of the ability of commercially available lateral flow assay strips to detect these labels.

When the resulting target nucleotide is intended for detection in a lateral flow assay, it may be preferably that the target nucleotide includes only a single label. In embodiments where the target nucleotide is intended for detection in a lateral flow assay and includes more than one label, the second label is preferably not a label that is detected by the lateral flow assay. In embodiments where the target polynucleotide is intended for detection in a lateral flow assay and includes more than one label, the second label is a label that is detected by the lateral flow assay. In embodiments where the target polynucleotide is intended for detection in a lateral flow assay and includes more than one label, the second label is a label that is detected by the lateral flow assay following Cas-based cleavage and uncoupling of biotin and FITC fragments that are observed at either the test or control line on an LFA.

In some embodiments, when the resulting target nucleotide is intended for detection in a fluorometric- or colorimetric-based assay, the label may be selected based on its ability to be detected in those assays. (See, e.g., Li et al., Trends in Biotechnology 2019; 37(7):730-743; Seamon et al. Anal. Chem. 2018; 90(11): 6913-6921; Chang et al., Microchimica Acta 2019; 186: 1-8.) For example, for a fluorometric- or colorimetric-based assay, the label may preferably be an enzyme/protein label.

Exposing the Target Nucleotide to a Cas Protein Such as Cas9 and a gRNA to Form a Target Nucleotide-Cas9 Complex

When the target nucleotide is exposed to Cas such as Cas9 and a gRNA, a target nucleotide-Cas9 complex may be formed.

In some embodiments, Cas9 and the gRNA may be allowed to form a (Cas9:gRNA) complex prior to the addition of the target nucleotide. In some embodiments, the gRNA and the target nucleotide may be added to the Cas9 at the same time. In some embodiments, the gRNA and the target nucleotide may be added to the Cas9 sequentially.

In some embodiments, the target nucleotide is exposed to Cas9 and the gRNA in the presence of competitor nucleic acid such as RNA, DNA, or cDNA that is single stranded, double stranded, linear, or circular. As described in Examples 6, 7, and 9, competitor DNA may be used to decrease or completely eliminate off-target/non-specific binding between the gRNA and a non-specific nucleotide.

In some embodiments, the competitor DNA is present in amount that provides a molar equivalent to the target nucleotide. In some embodiments, the competitor DNA is present in a molar excess to the target nucleotide. For example, the competitor DNA may be present in an amount of at least 1 fold, at least 2 fold, of at least 10 fold, of at least 100 fold, of at least 1,000 fold, of at least 2,000 fold, or of at least 10,000 fold the amount of the target nucleotide.

The competitor DNA preferably includes a PAM sequence. In some embodiments, the PAM may be a PAM from Neisseria meningitidis, Treponema denticola, Streptococcus thermophilus, Streptococcus pyogenes, Staphylococcus aureus, Francisella novicida, or Campylobacter jejuni, or Cas variants thereof, or a combination thereof (e.g., Cas 12, Cas13 etc). In some embodiments, the PAM in the competitor DNA is preferably a PAM recognized by the Cas9 being used to form the target nucleotide-Cas9 complex. For example, when the Cas9 being used to form the target nucleotide-Cas9 complex includes SpCas9, the PAM may include 5′-NGG-3′, wherein N is any nucleobase. In an exemplary embodiment, the PAM may include 5′-AGG-3′.

In an exemplary embodiment, the competitor DNA may include genomic DNA or an mCherry plasmid and/or a plasmid having a plasmid backbone with pcDNA3.1 being an example. mCherry includes both GGG or NGG PAM sequences.

In another exemplary embodiment, the competitor DNA may include an oligonucleotide including, for example, one or both of the oligonucleotides shown in Table 8A. In some embodiments, the oligonucleotide may lack homology to the target DNA. In some embodiments, the oligonucleotide preferably includes a PAM sequence.

In a further exemplary embodiment, the competitor DNA may include an oligonucleotide with homology (either partial or complete) to the target DNA, as described in an exemplary embodiment in Example 11.

In some embodiments, the target nucleotide may be exposed to Cas9 and gRNA in the presence of a binding salt such as NaCl. Without wishing to be bound by theory, it is believed that optimizing the salt concentration will decrease non-specific binding and promote more specific detection of the target nucleotide. Such specific detection could allow for target nucleotides having single base pair differences from the gRNA to be distinguished from target nucleotides having no base pair differences from the gRNA (that is, SNP detection).

In some embodiments, a concentration of at least of 0.1 M, at least 0.5 M, at least 1 M, or at least 2 M binding salt will be included. In some embodiments, a concentration of up to 0.5 M, up to 1 M, up to 2 M, up to 3 M, up to 4 M, or up to 5 M binding salt will be included.

### Detecting the Target Nucleotide-Cas9 Complex

The target nucleotide-Cas9 complex may be detected by any suitable means.

In some embodiments, detection of the nucleotide-Cas9 complex using a lateral flow assay (LFA) may be preferred. LFA is commonly used for rapid testing, such as home pregnancy tests, and does not require specialized infrastructure. To be used, the lateral flow assay must, however, be sensitive to the label included on the first primer and the label included on the Cas9.

In an exemplary embodiment, the LFA includes detecting an analyte labeled with biotin. For example, when the Cas9 is labeled with biotin, the LFA detects the biotin on the Cas9 in the target nucleotide-Cas9 complex.

In another exemplary embodiments, the LFA includes detecting an analyte labeled with fluorescein isothiocyanate (FITC), a fluorescein amidite (FAM), or digoxigenin (DIG). For example, when the first primer is labeled with FITC, FAM, or DIG, the LFA detects the FITC, FAM, or DIG on the target nucleotide in the target nucleotide-Cas9 complex.

Exemplary lateral flow assays include a PCRD nucleic acid lateral flow immunoassay (Abingdon Health, York, United Kingdom) and HybriDetect-Universal Lateral Flow Assay Kit (Milenia Hybritech, Gießen, Germany).

In some embodiments, the target nucleotide-Cas9 complex may be detected a fluorometric assay or a colorimetric assay. (See, e.g., Li et al., Trends in Biotechnology 2019; 37(7):730-743; Seamon et al. Anal. Chem. 2018; 90(11): 6913-6921; Chang et al., Microchimica Acta 2019; 186: 1-8.)

### Labeled Cas9 Composition Aspects

In another aspect, this disclosure describes a composition for use in a Cas9-based diagnostic assay.

The composition includes a labeled target nucleotide, a labeled Cas9 and a gRNA. In some embodiments, the composition includes a complex including the target nucleotide and the biotinylated Cas9. In some embodiments, the composition includes a complex including the target nucleotide, the biotinylated Cas9, and the gRNA.

When the composition is intended for detection in a lateral flow assay (LFA), the labels for the target nucleotide and the Cas9 may be selected based on the labels detectable by the LFA. In such embodiments, the Cas9 is preferably labeled with one of the labels used by an LFA test band and the target nucleotide is preferably labeled with the other of the labels used by an LFA test band. In such embodiments, the target nucleotide is preferably not labeled with the same label as the Cas9. In some embodiments, the target nucleotide includes only a single label.

In an exemplary embodiment, the labeled target nucleotide includes FITC, a fluorescein amidite (FAM), or digoxigenin (DIG), and the labeled Cas9 includes biotinylated Cas9. In another exemplary embodiment, the labeled target nucleotide includes biotin, a fluorescein amidite (FAM), or digoxigenin (DIG), and the labeled Cas9 includes FITC.

In some embodiments, the target nucleotide may include more than one target nucleotide. In some embodiments, different target nucleotides may be labeled with different labels.

In some embodiments, the composition may further include competitor DNA, as further described herein. The competitor DNA may be present in amount that provides a molar equivalent to the target nucleotide or in a molar excess to the target nucleotide. For example, the competitor DNA may present in an amount of at least 1 fold, at least 2 fold, at least 10 fold, at least 100 fold, at least 1,000 fold, at least 2,000 fold, or at least 10,000 fold the amount of the target nucleotide.

The labeled Cas9 may include any suitable Cas9. For example, the Cas9 may include, a Cas9 from Neisseria meningitidis, Treponema denticola, Streptococcus thermophilus, Streptococcus pyogenes, Staphylococcus aureus, Francisella novicida, or Campylobacter jejuni, or a variant thereof, or a combination thereof. In some embodiments, the variant may preferably increase specificity; for example, SpyFi Cas9 (Aldevron, Fargo, N. Dak.). In an exemplary embodiment, the Cas9 includes Streptococcus pyogenes Cas9 (SpCas9). In another exemplary embodiment, the Cas9 includes a variant of Streptococcus pyogenes Cas9 (SpCas9) including, for example, SpyFi Cas9 (Aldevron, Fargo, N. Dak.).

In some embodiments, the target nucleotide preferably includes a protospacer adjacent motif (PAM). In some embodiments, if the nucleotide to be amplified did not include a PAM (or does not include an effective PAM), a PAM may be introduced into the target nucleotide using specially designed primers for amplification of the nucleotide to form the target nucleotide.

The PAM may include a PAM from Neisseria meningitidis, Treponema denticola, Streptococcus thermophilus, Streptococcus pyogenes, Staphylococcus aureus, Francisella novicida, or Campylobacter jejuni, or a variant thereof, or a combination thereof. In some embodiments, the PAM in the target nucleotide preferably includes a PAM recognized by the Cas9 being used to form the target nucleotide-Cas9 complex. For example, when the Cas9 being used to form the target nucleotide-Cas9 complex includes SpCas9, the PAM in the target nucleotide may include 5′-NGG-3′, wherein N is any nucleobase. In an exemplary embodiment, the PAM may include 5′-AGG-3′.

### Single and Multiplexed Targets

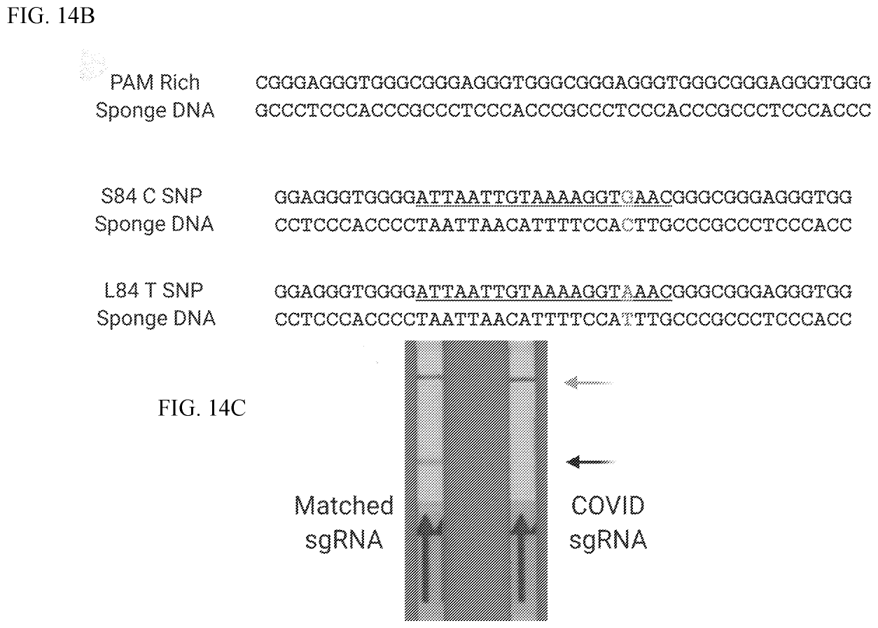

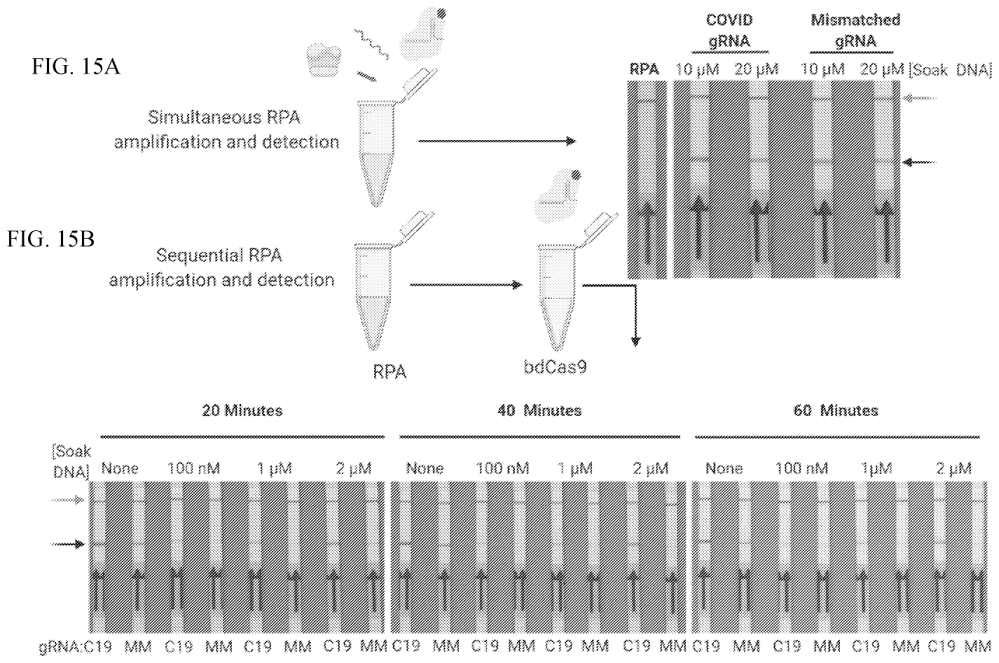

The nuclease properties of Cas9 can also be used to serve as a diagnostic platform by cleaving a fluorescent probe in a sequence-specific manner (FIG. 14A). COVID-19-specific probes labeled with a fluorescent marker and quencher can be utilized, that when hybridized with SARS-CoV-2 (for example) amplicons, can be cleaved by Cas9 nuclease together with a COVID-19 sgRNA (FIG. 14B).

The ability to multiplex Cas9 with multiple sgRNAs also allowed simultaneous detection of viruses with overlapping symptomology. First, DNA probes were designed and built with distinct fluorophores for SARS-CoV-2, influenza A and B, and RSV, respectively. These probes were tested and showed specificity of fluorescent signaling only for matched sgR-NAs (FIG. 15A,B). Additionally, or optionally, the four distinct viral detection components could be combined in a single reaction mixture and analyzed simultaneously under isothermal (37° C.) conditions in a single tube using real-time fluorometry with a standard quantitative PCR instrument in a 96-well format (FIG. 15C). Distinct fluorescence signals from cleavage of the pathogen-specific probes by the disease-specific sgRNAs were observed. These results showed that all four viral pathogens could be detected in a multiplex fashion. In some embodiments, steps to detect other targets, additional targets, or combinations thereof could also be included in disclosed methods.

Disclosed methods can offer limits of detection (LOD) that represents not more than 5 copies of an original starting material.

### Single Nucleotide Specificity

Soak DNA can be utilized in some embodiments. Soak DNA, which can also be described as bait DNA can include a sequence comprised of PAM-rich sequences that are designed for inclusion in an assay in order to sequester non-specific binding events. When such soak DNA sequences are utilized, positive results may be observed when the target is present, but not similar sequences. In some embodiments, the amount of time that the soak DNA is incubated in contact with the target can affect the resolution. For example, the soak DNA can be incubated in contact with the target for at least 10 minutes, at least 15 minutes, at least 20 minutes, at least 25 minutes, at least 30 minutes, or at least 35 minutes. For example, the soak DNA can be incubated in contact with the target for not more than 90 minutes, not more than 75 minutes, not more than 70 minutes, not more than 65 minutes, not more than 60 minutes, not more than 55 minutes, or not more than 50 minutes.

In some embodiments where nuclease-dependent fluorescence is utilized, soak DNA may not be necessary for higher degrees of specificity.

### Additional Considerations

In some embodiments, a human (for example) control gene can be differentially labeled to allow for viral target and control genes to be analyzed using a single LFA, for example.

In some embodiments, methods disclosed herein could be utilized to obtain information on viral polymorphisms. Such information could be utilized to track and/or monitor the spread, infectivity, or both of viral strains (e.g., COVID strains).

### Exemplary Labeled Cas9 Diagnostic Method Aspects

1. A method comprising

amplifying a nucleotide to form a target nucleotide, wherein the amplification comprises using a first primer and a second primer, wherein the first primer comprises a label;

exposing the target nucleotide to Cas9 and a gRNA, wherein the Cas9 comprises a label, to form a target nucleotide-Cas9 complex; and

detecting the target nucleotide-Cas9 complex in a lateral flow assay, a fluorometric assay, or a colorimetric assay.

2. The method of Aspect 1, wherein the second primer is not labeled with biotin.
3. The method of Aspect 1 or Aspect 2, wherein the second primer is not labeled.
4. The method of any one of the preceding Aspects, wherein the Cas9 comprises biotinylated Cas9.
5. The method of any one of the preceding Aspects, wherein amplifying the nucleotide comprises recombinase polymerase amplification (RPA).
6. The method of any one of the preceding Aspects, wherein the label of the first primer comprises FITC, a fluorescein amidite (FAM), or digoxigenin (DIG).
7. The method of any one of the preceding Aspects, wherein the method further comprises exposing the target nucleotide to Cas9 and a gRNA in the presence of competitor DNA.
8. The method of Aspect 7, wherein the competitor DNA comprises mCherry or an oligonucleotide.
9. The method of Aspect 7 or Aspect 8, wherein the competitor DNA comprises a protospacer adjacent motif (PAM).
10. The method of any one of Aspects 7 to 9, wherein the competitor DNA is present in a molar excess to the target nucleotide or wherein the competitor DNA is present in amount that provides a molar equivalent to the target nucleotide.
11. The method of Aspect 10, wherein the competitor DNA is present in an amount of at least 1 fold, at least 2 fold, at least 10 fold, at least 100 fold, at least 1,000 fold, at least 2,000 fold, or at least 10,000 fold the amount of the target nucleotide.
12. The method of any one of the preceding Aspects, the method further comprises forming a Cas9:gRNA complex prior to exposing the target nucleotide to Cas9 and the gRNA.
13. The method of any one of the preceding Aspects, wherein method comprises detecting the target nucleotide-Cas9 complex in a lateral flow assay, wherein the lateral flow assay comprises detecting an analyte labeled with biotin and FITC, a fluorescein amidite (FAM), or digoxigenin (DIG).
14. The method of any one of the preceding Aspects, wherein the nucleotide to be amplified comprises RNA, DNA, or cDNA.
15. The method of any one of the preceding aspects wherein the Cas9 comprises Cas9 from Neisseria meningitidis, Treponema denticola, Streptococcus thermophilus, Streptococcus pyogenes, Staphylococcus aureus, Francisella novicida, or Campylobacter jejuni, or a variant thereof.
16. The method of any one of the preceding aspects wherein the Cas9 comprises Streptococcus pyogenes Cas9 (SpCas9).
17. The method of any one of the preceding Aspects, wherein the target nucleotide comprises a protospacer adjacent motif (PAM).
18. The method of Aspect 17, wherein the protospacer adjacent motif (PAM) comprises 5′-NGG-3′, wherein N is any nucleobase.
19. The method of Aspect 18, wherein the PAM comprises 5′-AGG-3′.
20. The method of any one of the preceding Aspects, wherein the method comprises detecting Influenza.
21. The method of any one of the preceding Aspects, wherein the method comprises detecting RSV.
22. The method of any one of the preceding Aspects, wherein the method comprises distinguishing a single nucleotide polymorphism from another.
23. The method of any of Aspects 7 to 11, wherein the method comprises distinguishing a single nucleotide polymorphism from another.

### Exemplary SARS-CoV-2-Specific Method Aspects

1. The method of any one of the Labeled Cas9 Diagnostic Method Aspects, wherein the method comprises detecting SARS-CoV-2.
2. The method of Aspect 1, wherein the target nucleotide comprises a SARS-CoV-2-specific sequence.
3. The method of Aspect 1 or 2, wherein the gRNA comprises a SARS-CoV-2-specific sequence.
4. The method of any one of the preceding Aspects, wherein the target nucleotide comprises a sequence from ORF1ab or ORF8.
5. The method of any one of the preceding Aspects, wherein the target nucleotide comprises

(SEQ ID NO: 9)
ATTAATTGTAAAAGGTAAAC,
(SEQ ID NO: 10),
ATTAATTGTAAAAGGTGAAC,
(SEQ ID NO: 11)
ATTAATTGTAAAAGGTAAACAGG,
or
(SEQ ID NO: 12)
ATTAATTGTAAAAGGTGAACAGG.

6. The method of any one of the preceding Aspects, wherein the gRNA comprises a sequence from ORF1ab or ORF8.
7. The method of any one of the preceding Aspects, wherein the gRNA comprises

(SEQ ID NO: 7)
AUUAAUUGUAAAAGGUGAAC (COVID 28112C),
or
(SEQ ID NO: 8)
AUUAAUUGUAAAAGGUAAAC (COVID 28112T).

8. The method of any one of the preceding Aspects, wherein the first primer comprises taacaaacatgctgattttgacacatgg (SEQ ID NO: 1) (ORF1ab forward primer) and/or the second primer comprises ccaggcacgacaaaacccac (SEQ ID NO: 2) (ORF1ab reverse primer).
9. The method of any one of the preceding Aspects, wherein the first primer comprises ctaaatcacccattcagtacatcgatatcg (SEQ ID NO: 3) (ORF8 forward primer) and/or the second primer comprises cttcatagaacgaacaacgcactacaagactacc (SEQ ID NO: 4) (ORF8 reverse primer).
10. The method of any one of the preceding Aspects, wherein the first primer comprises gaattgtgcgtggatgaggctgg (SEQ ID NO: 5) (28112 forward primer) and/or the second primer comprises caacacgaacgtcatgatactc (SEQ ID NO: 6) (28112 reverse primer).
11. The method of any one of the preceding Aspects, wherein the first primer comprises ctaaatcacccattcagtacatcgatatcg (SEQ ID NO: 3) (28112 forward primer) and/or the second primer comprises caacacgaacgtcatgatactc (SEQ ID NO: 6) (28112 reverse primer).
12. The method of any one of the preceding Aspects, wherein the method comprises detecting Influenza.
13. The method of any one of the preceding Aspects, wherein the method comprises detecting RSV.
14. The method of any one of the preceding Aspects, wherein the method comprises distinguishing a single nucleotide polymorphism from another.

### Exemplary Influenza-Specific Method Aspects

1. The method of any one of the Labeled Cas9 Diagnostic Method Aspects, wherein the method comprises detecting an Influenza virus.
2. The method of Aspect 1, wherein the target nucleotide comprises an Influenza A-specific sequence.
3. The method of Aspect 1 or 2, wherein the gRNA comprises an Influenza B-specific sequence.
4. The method of any one of the preceding Aspects, wherein the target nucleotide comprises a sequence from ORF1ab or ORFS.
5. The method of any one of the preceding Aspects, wherein the target nucleotide comprises

(SEQ ID NO: 9)
ATTAATTGTAAAAGGTAAAC,
(SEQ ID NO: 10),
ATTAATTGTAAAAGGTGAAC,
(SEQ ID NO: 11)
ATTAATTGTAAAAGGTAAACAGG,
or
(SEQ ID NO: 12)
ATTAATTGTAAAAGGTGAACAGG.

6. The method of any one of the preceding Aspects, wherein the gRNA comprises a sequence from ORF1ab or ORF8.
7. The method of any one of the preceding Aspects, wherein the gRNA comprises

(SEQ ID NO: 7)
AUUAAUUGUAAAAGGUGAAC (COVID 28112C),
or
(SEQ ID NO: 8)
AUUAAUUGUAAAAGGUAAAC (COVID 28112T).

8. The method of any one of the preceding Aspects, wherein the first primer comprises taacaaacatgctgattttgacacatgg (SEQ ID NO: 1) (ORF1ab forward primer) and/or the second primer comprises ccaggcacgacaaaacccac (SEQ ID NO: 2) (ORF1ab reverse primer).
9. The method of any one of the preceding Aspects, wherein the first primer comprises ctaaatcacccattcagtacatcgatatcg (SEQ ID NO: 3) (ORF8 forward primer) and/or the second primer comprises cttcatagaacgaacaacgcactacaagactacc (SEQ ID NO: 4) (ORF8 reverse primer).
10. The method of any one of the preceding Aspects, wherein the first primer comprises gaattgtgcgtggatgaggctgg (SEQ ID NO: 5) (28112 forward primer) and/or the second primer comprises caacacgaacgtcatgatactc (SEQ ID NO: 6) (28112 reverse primer).
11. The method of any one of the preceding Aspects, wherein the first primer comprises ctaaatcacccattcagtacatcgatatcg (SEQ ID NO: 3) (28112 forward primer) and/or the second primer comprises caacacgaacgtcatgatactc (SEQ ID NO: 6) (28112 reverse primer).
12. The method of any one of the preceding Aspects, wherein the method comprises detecting Influenza.
13. The method of any one of the preceding Aspects, wherein the method comprises detecting RSV.
14. The method of any one of the preceding Aspects, wherein the method comprises distinguishing a single nucleotide polymorphism from another.

### Exemplary Labeled Cas9 Composition Aspects

1. A composition comprising

a target nucleotide, wherein the target nucleotide comprises a label;

a labeled Cas9, a nuclease Cas9, a nickase Cas9, or any combination thereof;

and a gRNA.

2. The composition of Aspect 1, wherein the label of the target nucleotide comprises FITC, a fluorescein amidite (FAM), or digoxigenin (DIG), and wherein the labeled Cas9 comprises biotinylated Cas9.
3. The composition of Aspect 1 or Aspect 2, wherein the target nucleotide comprises more than one target nucleotide.
4. The composition of any one of the preceding Aspects, wherein the target nucleotide comprises a single label.
5. The composition of any one of the preceding Aspects, wherein the composition further comprises competitor DNA.
6. The composition of Aspect 5, wherein the competitor DNA comprises mCherry or an oligonucleotide.
7. The composition of Aspect 4 or Aspect 5, wherein the competitor DNA is present in amount that provides a molar equivalent to the target nucleotide or wherein the competitor DNA is present in a molar excess to the target nucleotide.
8. The composition of Aspect 7, wherein the competitor DNA is present in an amount of at least 1 fold, at least 2 fold, at least 10 fold, at least 100 fold, at least 1,000 fold, at least 2,000 fold, or at least 10,000 fold the amount of the target nucleotide.
9. The composition of any one of the preceding Aspects wherein the labeled Cas9 comprises Cas9 from Neisseria meningitidis, Treponema denticola, Streptococcus thermophilus, Streptococcus pyogenes, Staphylococcus aureus, Francisella novicida, or Campylobacter jejuni, or a variant thereof.
10. The composition of any one of the preceding aspects wherein the labeled Cas9 comprises Streptococcus pyogenes Cas9 (SpCas9).
11. The composition of any one of the preceding Aspects, wherein the target nucleotide comprises a protospacer adjacent motif (PAM).
12. The composition of Aspect 11, wherein the protospacer adjacent motif (PAM) comprises 5′-NGG-3′, wherein N is any nucleobase.
13. The composition of any one of the preceding Aspects, wherein the composition comprises a complex comprising the target nucleotide and the labeled Cas9.
14. The composition of Aspect 13, the complex further comprising the gRNA.
15. The composition of any one of the preceding Aspects, wherein the target nucleotide comprises an Influenza specific sequence.
16. The composition of any one of the preceding Aspects, wherein the target nucleotide comprises a RSV specific sequence.
17. The composition of any one of the preceding Aspects, wherein there is at least a single nucleotide difference from one target nucleotide and another target nucleotide.
18. The composition of any of Aspects 5 to 8, wherein there is at least a single nucleotide difference from one target nucleotide and another target nucleotide.

### Exemplary Methods of Using the Composition Aspects

1. A method comprising detecting the complex of Aspect 13 or 14 of the Exemplary Labeled Cas9 Composition Aspects.
2. The method of Aspect 1, wherein the method comprises detecting the complex using a lateral flow assay.
3. The method of Aspect 1, wherein the method comprises detecting a complex using a fluorometric or colorimetric assay.

### Exemplary SARS-CoV-2-Specific Composition Aspects:

1. The composition of any one of the Exemplary Labeled Cas9 Composition Aspects, wherein the target nucleotide comprises a SARS-CoV-2-specific sequence.
2. The composition of Aspect 1, wherein the gRNA comprises a SARS-CoV-2-specific sequence.
3. The composition of Aspect 1 or 2, wherein the target nucleotide comprises a sequence from ORF1ab or ORF8.
4. The composition of any one of the preceding Aspects, wherein the target nucleotide comprises

(SEQ ID NO: 9)
ATTAATTGTAAAAGGTAAAC,
(SEQ ID NO: 10),
ATTAATTGTAAAAGGTGAAC,
(SEQ ID NO: 11)
ATTAATTGTAAAAGGTAAACAGG,
or
(SEQ ID NO: 12)
ATTAATTGTAAAAGGTGAACAGG.

5. The composition of any one of the preceding Aspects, wherein the gRNA comprises a sequence from ORF1ab or ORF8.
6. The composition of any one of the preceding Aspects, wherein the gRNA comprises

(SEQ ID NO: 7)
AUUAAUUGUAAAAGGUGAAC (COVID 28112C),
or
(SEQ ID NO: 8)
AUUAAUUGUAAAAGGUAAAC (COVID 28112T).

7. The composition of any one of the preceding Aspects, wherein the first primer comprises taacaaacatgctgattttgacacatgg (SEQ ID NO: 1) (ORF1ab forward primer) and/or the second primer comprises ccaggcacgacaaaacccac (SEQ ID NO: 2) (ORF1ab reverse primer).
8. The composition of any one of the preceding Aspects, wherein the first primer comprises ctaaatcacccattcagtacatcgatatcg (SEQ ID NO: 3) (ORF8 forward primer) and/or the second primer comprises cttcatagaacgaacaacgcactacaagactacc (SEQ ID NO: 4) (ORF8 reverse primer).
9. The composition of any one of the preceding Aspects, wherein the first primer comprises gaattgtgcgtggatgaggctgg (SEQ ID NO: 5) (28112 forward primer) and/or the second primer comprises caacacgaacgtcatgatactc (SEQ ID NO: 6) (28112 reverse primer).
10. The composition of any one of the preceding Aspects, wherein the first primer comprises ctaaatcacccattcagtacatcgatatcg (SEQ ID NO: 3) (28112 forward primer) and/or the second primer comprises caacacgaacgtcatgatactc (SEQ ID NO: 6) (28112 reverse primer).

The present invention is illustrated by the following examples. It is to be understood that the particular examples, materials, amounts, and procedures are to be interpreted broadly in accordance with the scope and spirit of the invention as set forth herein.

### EXAMPLES

All reagents, starting materials, and solvents used in the following examples were purchased from commercial suppliers (such as Sigma Aldrich, St. Louis, Mo.) and were used without further purification unless otherwise indicated.

### Example 1

This Example describes the amplification of SARS-CoV-2 DNA or reverse transcribed RNA Ultramers. The RNA or DNA Ultramers included nucleotide sequences from Open Reading Frame 1 (ORF1ab) or Open Reading Frame 8 (ORF8), as shown in Table 1A.

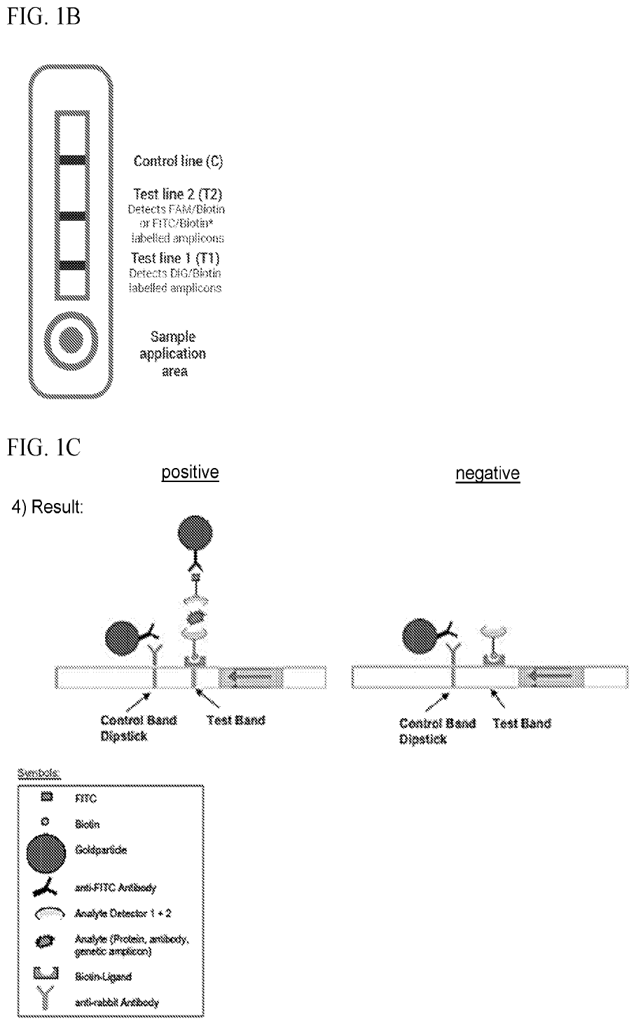

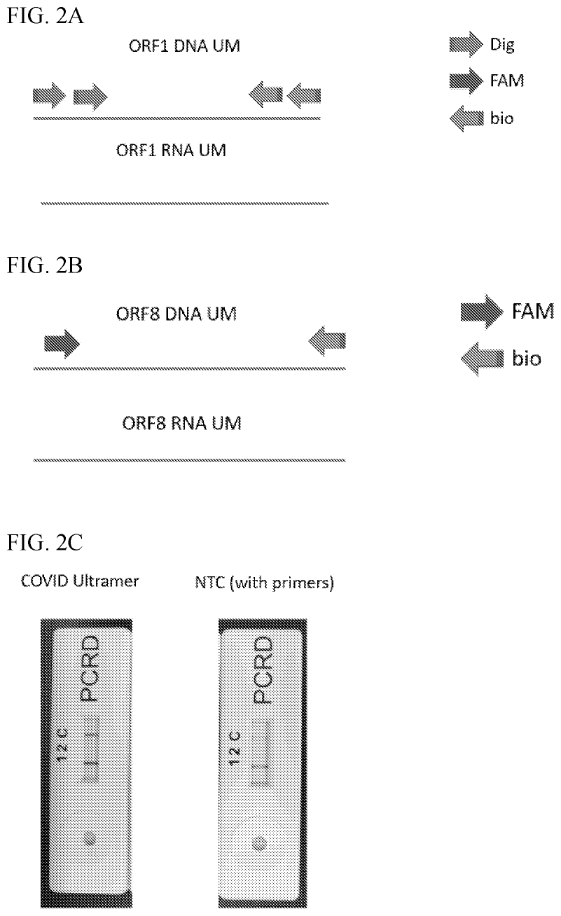

The Ultramers were amplified with a DIG-conjugated forward primer (FIG. 2A, blue arrow; ORF1ab amplicon) or FAM/FITC-conjugated forward primer for the ORF8 amplicon (FIG. 2B, purple arrow) and a biotinylated reverse primer (FIG. 2A and FIG. 2B, red arrow) using end point PCR with Phusion polymerase (ThermoFisher Scientific, Waltham, Mass.) or TwistDx's isothermal nucleic acid amplification technology, recombinase polymerase amplification (RPA) (TwistDx, Cambridge, United Kingdom).

Schematics of the Ultramers from Integrated DNA Technologies (IDT, Coralville, Iowa) are shown in FIG. 2A and FIG. 2B. Sequences of the Ultramers are shown in Table 1A; sequences of the primers are shown in Table 1B.

COVID-19 Ultramers (see Table 1A) were amplified with a FAM/FITC (ORFS) or DIG (ORF1ab) conjugated forward primer (see Table 1B) and a biotinylated reverse primer (see Table 1B) using TWISTDx RPA. The results were run on a PCRD nucleic acid lateral flow immunoassay (Abingdon Health, York, United Kingdom) (see FIG. 1B). Results are shown in the left panel of FIG. 2C.

A no-template control (NTC) was also prepared. In the NTC, the RPA mixture did not include a DNA or RNA Ultramer template—but primers were included. Results are shown in the right panel of FIG. 2C. The presence of a band at test line 1 is a false positive in the no-template control.

TABLE 1A

| | | | SEQ |
|---|---|---|---|
| Ultramer | | | ID |
| (Template) | Sequence (Red indicates reported polymorphism) | nt | NO: |
| ORF1ab | gtcactcgtgacatagcatctacagatacttgttttgctaacaaacatgctgattttgacac | 184 | 13 |
| DNA | atggtttagCcagcgtggtggtagttatactaatgacaaagcttgcccattgattgctgca | | |
| Ultramer | gtcataacaagagaagtgggttttgtcgtgcctggtttgcctggcacgatattacgcacaa | | |
| ORF1ab | UAACAAACAUGCUGAUUUUGACACAUGGUUUAGCCAGC | 120 | 14 |
| RNA | GUGGUGGUAGUUAUACUAAUGACAAAGCUUGCCCAUUG | | |
| Ultramer | AUUGCUGCAGUCAUAACAAGAGAAGUGGGUUUUGUCGU | | |
| | GCCUGG | | |
| ORF8 DNA | gaattgtgcgtggatgaggctggttctaaatcacccattcagtacatcgatatcggtaatta | 175 | 15 |
| Ultramer | tacagtttcctgttTaccttttacaattaattgccaggaacctaaattgggtagtcttgtagt | | |
| | gcgttgttcgttctatgaagactttttagagtatcatgacgttcgtgttg | | |
| ORF8 RNA | CUAAAUCACCCAUUCAGUACAUCGAUAUCGGUAAUUAU | 120 | 16 |
| Ultramer | ACAGUUUCCUGUUUACCUUUUACAAUUAAUUGCCAGGA | | |
| | ACCUAAAUUGGGUAGUCUUGUAGUGCGUUGUUCGUUCU | | |
| | AUGAAG | | |

TABLE 1B

| Primer | Sequence | SEQ ID NO: |
|---|---|---|
| DIG-conjugated Forward | /5DigN/taacaaacatgctgattttgacacatgg | 17 |
| Primer (ORF1ab) | | |
| Biotin-conjugated | 5Biosg/ccaggcacgacaaaacccac | 18 |
| Reverse Primer | | |
| (ORF1ab) | | |
| FAM-conjugated | /56-FAM/ctaaatcacccattcagtacatcgatatcg | 19 |
| Forward Primer (ORF8) | | |
| Biotin-conjugated | 5Biosg/cttcatagaacgaacaacgcactacaagactacc | 20 |
| Reverse Primer (ORF8 | | |
| RNA) | | |

### Example 2

This Example describes RT-PCR of a SARS-CoV-2 RNA Ultramer or PCR of a DNA Ultramer (as described in Example 1) to determine if primers were forming dimers.

The RNA Ultramer ORF1 or ORF8 was spiked (at a 2 μM concentration) into 18 (10 ng/ul) of human HEK 293 cDNA and then amplified via endpoint PCR using RT 8750 RNA Dig RT F; RT 8750 RNA RT REV or RT 28112 RNA RT FAM F; RT 28112 RNA RT REV using Phusion polymerase with GC buffer (ThermoFisher Scientific, Waltham, Mass.): 98 C×30 s; 98×10 s; 62×30 s; 72×15 s×35 cycles.

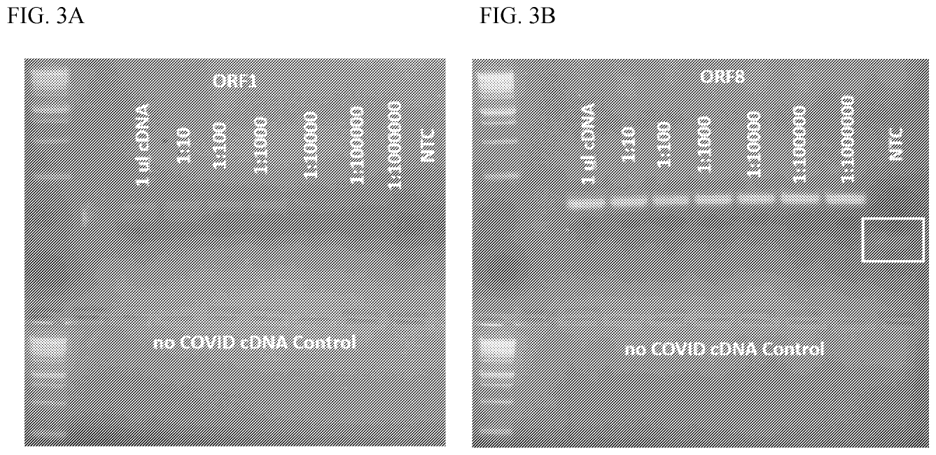

Serial dilutions were prepared and resolved on an agarose gel. Results are shown in FIG. 3A (ORF1ab) and FIG. 3B (ORF8). The right most lane shows the no template control. The bottom gel in each panel shows human HEK 293 cDNA without COVID ultramer.

The yellow box in FIG. 3B indicates the presence of primer dimers.

### Example 3

This Example describes dilution of primers to minimize primer dimerization.

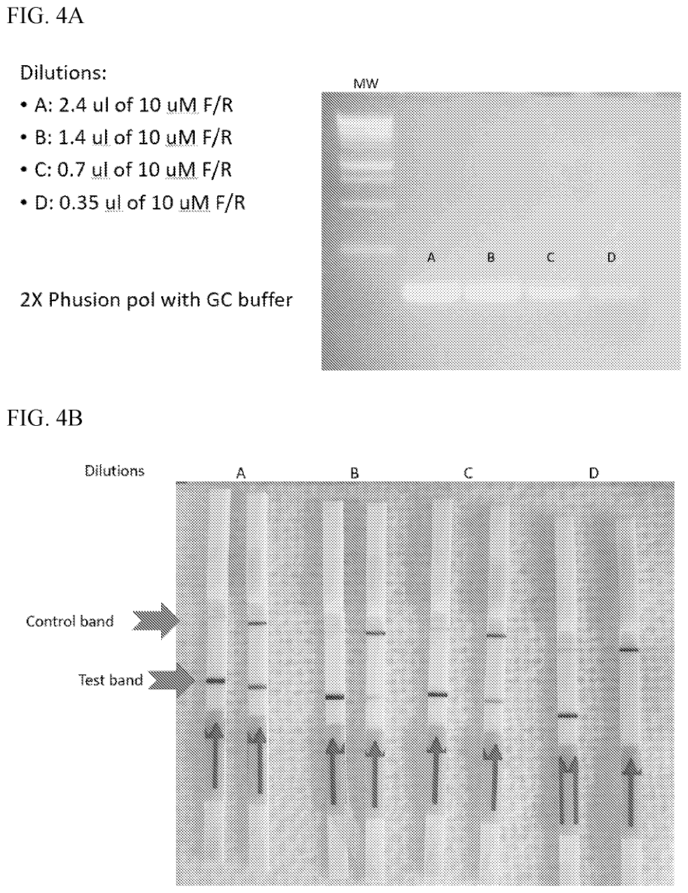

The 28112 primers RT 28112 RNA RT FAM F; RT 28112 RNA RT REV (sequences in Table 2) were diluted serially at the indicated concentrations and then used in an endpoint PCR with Phusion GC as described in Example 2 and resolved on an agarose gel. Results are shown in FIG. 4A.

The PCR products (22 μL) were mixed with 100 μl of flow buffer (MILENIA01, Milenia Hybritech, Gießen, Germany) and resolved via LFA (HybriDetect-Universal Lateral Flow Assay Kit, Milenia Hybritech, Gießen, Germany) (see FIG. 1C).

Results are shown in FIG. 4B. For each dilution A-D, the left strip shows PCR with template (Ultramer) and the right strip shows the no template (Ultramer) control. In FIG. 4B, at dilutions A-C, the strips with no template showed a product at the test band (lower arrow) —indicating a false positive. At the lowest primer concentration (dilution D), the strip with no template did not show a product at the test band (lower arrow).

TABLE 2

| | |
|---|---|
| RT 28112 RNA RT FAM F | /56-FAM/ctaaatcacccattcagtacatcgatatcg (SEQ ID NO: 19) |
| RT 28112 RNA RT REV (bio) | 5Biosg/cttcatagaacgaacaacgcactacaagactacc (SEQ ID NO: 20) |

### Example 4

This example describes the result of an LFA assay for SARS-CoV-2 in which the amplification step used one labeled primer and one unlabeled primer. Without the use of a second primer, the amplification product would normally be undetectable via an LFA devices in which DIG or FAM is captured and biotin is used for visualization. Detection of the amplification product was restored by including a biotinylated Cas9 and a guide RNA (gRNA) specific for the amplification product or with one base pair variation from the amplification product.

Target DNA from a synthetic COVID-19 Ultramer was amplified using end-point PCR with Phusion GC as described in Example 2. The sequence of the Ultramer is shown in Table 3A. The PCR forward primer was labeled with FAM and the reverse primer was unlabeled. The sequences of the primers are shown in Table 3B.

The PCR product was excised and quantified (37 ng/μL). The sequence of the target DNA—that is, the sequence of COVID-19 that is targeted by the sgRNA—is shown in Table 3C (with the canonical protospacer adjacent motif (PAM) of Streptococcus pyogenes Cas9 (SpCas9), 5′-NGG-3′, shown in bold). The PCR product was ˜150 bp and 23 bp of that is targeted/bound by Cas9.

3.5 μL of the PCR product was then mixed with biotinylated Cas9 (dCas9-3×FLAG-Biotin Protein, Sigma Aldrich, St. Louis, Mo.) and a gRNA, or with water, or with a dilution buffer provided with the biotinylated Cas9, as further described in Table 3E. Sequences of the gRNAs (ORF8 gRNAs: 28112C or 28112T) are shown in Table 3D. Reactions were incubated at 37° C. for one hour.

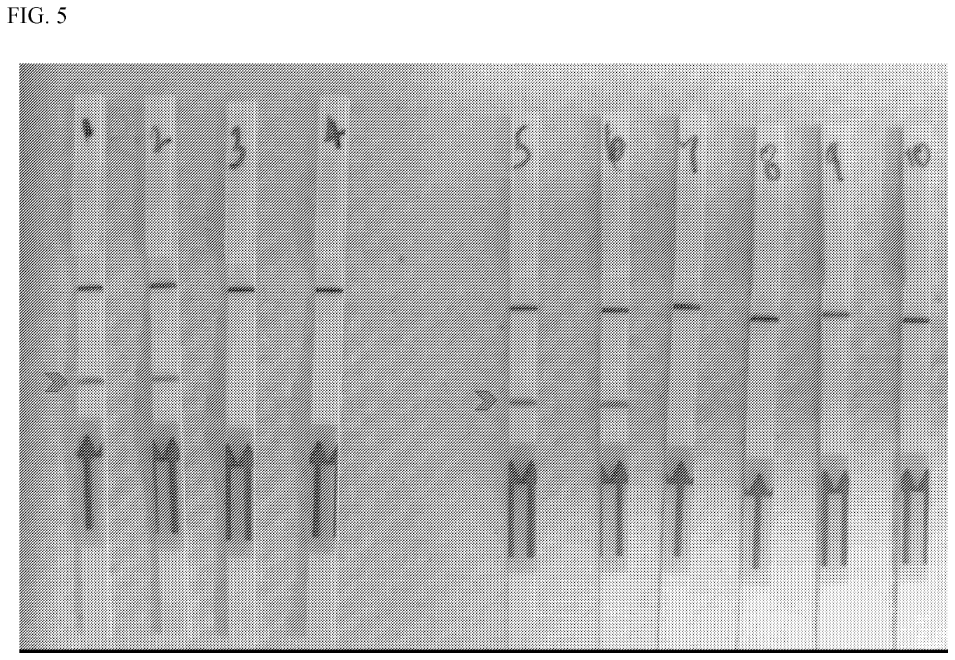

Reaction components were then resolved by LFA (HybriDetect-Universal Lateral Flow Assay Kit, Milenia Hybritech, Gießen, Germany) (see FIG. 1C). Results are shown in FIG. 5.

Conditions 1 and 2 (see Table 3E) which included PCR product, COVID gRNA 28112C (which exhibited a 1 base mismatch with the PCR product (target), and biotinylated Cas9, exhibited positive test bands, indicated by the blue arrows in FIG. 5.

Conditions 3 and 4 (see Table 3E) which included COVID gRNA 28112C, and biotinylated Cas9, but no PCR product did not exhibit positive test bands.

Conditions 5 and 6 (see Table 3E) which included PCR product, COVID gRNA 28112T (which exhibited a perfect match with the PCR product (target)), and biotinylated Cas9, exhibited positive test bands, indicated by the blue arrows in FIG. 5.

Conditions 7 and 8 (see Table 3E) which included COVID gRNA 28112T, and biotinylated Cas9, but no PCR product did not exhibit positive test bands.

Condition 8 which includes PCR product and water did not exhibit a positive test band. Condition 9 which includes PCR product and dilution buffer did not exhibit a positive test band.

TABLE 3A

| DNA Ultramer | | SEQ ID |
|---|---|---|
| (Template) | Sequence | NO: |
| 28112 DNA | gaattgtgcgtggatgaggctggttctaaatcacccattcagtacatcgatatcggtaattatacagttt | 15 |
| UM | cctgttTaccttttacaattaattgccaggaacctaaattgggtagtcttgtagtgcgttgttcgttctat | |
| | gaagactttttagagtatcatgacgttcgtgttg | |
| RNA Ultramer | | |
| (Template) | | |
| 28112 RNA UM | CUAAAUCACCCAUUCAGUACAUCGAUAUCGGUAAUUAUA | 16 |
| | CAGUUUCCUGUUUACCUUUUACAAUUAAUUGCCAGGAAC | |
| | CUAAAUUGGGUAGUCUUGUAGUGCGUUGUUCGUUCUAUG | |
| | AAG | |

TABLE 3B

| Primer (DNA) | Sequence | SEQ ID NO: |
|---|---|---|
| 28112 FAM-conjugated | /56-FAM/Gaattgtgcgtggatgaggctgg | 21 |
| Forward Primer | | |
| 28112 Unconjugated Reverse | caacacgaacgtcatgatactc | 6 |
| Primer | | |
| Primer (RNA/RT-PCR) | | |
| 28112 FAM-conjugated | /56-FAM/ctaaatcacccattcagtacatcgatatcg | 19 |
| Forward Primer | | |
| 28112 Unconjugated Reverse | caacacgaacgtcatgatactc | 6 |
| Primer | | |

TABLE 3C

| | |
|---|---|
| ″Target″ | ATTAATTGTAAAAGGTAAAC AGG |
| (anti-sense strand of COVID ORF1ab) | (SEQ ID NO: 11) |

TABLE 3D

| gRNA | Sequence | SEQ ID NO: |
|---|---|---|
| COVID 28112C | AUUAAUUGUAAAAGGUGAAC | 7 |
| COVID 28112T | AUUAAUUGUAAAAGGUAAAC | 8 |

TABLE 3E
Table 3E discloses ″AUUAAUUGUAAAAGGUGAAC″ as SEQ ID NO: 7 and
″AUUAAUUGUAAAAGGUAAAC″ as SEQ ID NO: 8.
1. 3.5 ul DNA + 1 ul Cas9/gRNA COVID 28112 C. AUUAAUUGUAAAAGGUGAAC
2. 3.5 ul DNA + 1 ul Cas9/gRNA COVID 28112 C. AUUAAUUGUAAAAGGUGAAC + 5.5 ul Dilution
buffer
3. 1 ul Cas9/gRNA COVID 28112 C. AUUAAUUGUAAAAGGUGAAC + 9 ul water
4. 1 ul Cas9/gRNA COVID 28112 C. AUUAAUUGUAAAAGGUGAAC + 9 Dilution buffer
5. 3.5 ul DNA + 1 ul Cas9/gRNA COVID 28112 T. AUUAAUUGUAAAAGGUAAAC
6. 3.5 ul DNA + 1 ul Cas9/gRNA COVID 28112 T. AUUAAUUGUAAAAGGUAAAC + 5.5 ul Dilution
buffer
7. 1 ul Cas9/gRNA COVID 28112 T. AUUAAUUGUAAAAGGUAAAC + 9 ul water
8. 1 ul Cas9/gRNA COVID 28112 T. AUUAAUUGUAAAAGGUAAAC + 9 Dilution buffer
9. 3.5 ul DNA + 6.5 ul HOH
10. 3.5 ul DNA + 6.5 ul Dilution buffer

### Example 5

This Example describes amplification of a target DNA from a synthetic COVID-19 Ultramer, as described in Example 4; and additionally, demonstrates that a shorter RNA product could be detected in the same manner following reverse transcription. RNA Ultramers can only be synthesized at a smaller length than DNA due to technological hurdles inherent to current synthetic nucleic acid platforms; so it was desired to validate that a smaller nucleic acid (i.e., RNA) target amplifies as efficiently as the longer DNA target and can be detected at the same level.

Target DNA from a synthetic COVID-19 Ultramer was amplified using end-point PCR with Phusion GC as described in Example 2. The sequence of the Ultramer is shown in Table 3A. The PCR forward primer was labeled with FAM and the reverse primer was unlabeled. The sequences of the primers are shown in Table 3B.

The PCR product (Target DNA) was excised and quantified (47 ng/μL). The sequence of the target DNA is shown in Table 3C.

3.5 μL of the PCR product was mixed with biotinylated Cas9 (dCas9-3×FLAG-Biotin Protein, Sigma Aldrich, St. Louis, Mo.) and a gRNA, as further described in Table 4 (Conditions 7 and 8), to provide a positive control. 3.5 μL of the PCR product was mixed with biotinylated Cas9 (no guide RNA) and dilution buffer to serve as a negative control, as further described in Table 4 (Condition 9).

Sequences of the gRNAs (28112C or 28112T) are shown in Table 3D.

Reactions were incubated at 37° C. for one hour.

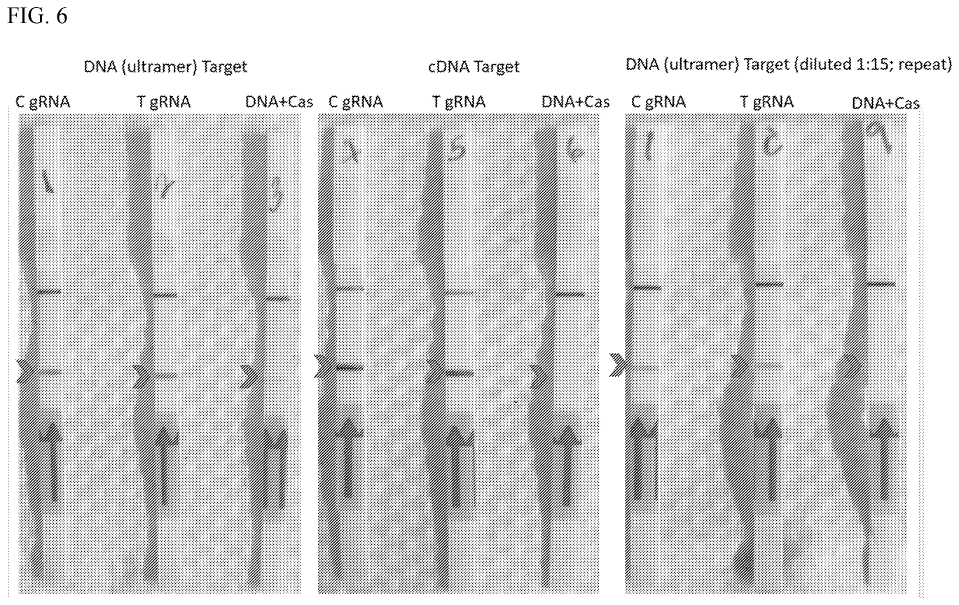

Reaction components were then resolved by LFA (HybriDetect-Universal Lateral Flow Assay Kit, Milenia Hybritech, Gießen, Germany) (see FIG. 1C). Results are shown in FIG. 6.

TABLE 4
Table 4 discloses ″AUUAAUUGUAAAAGGUGAAC″ as SEQ ID NO: 7
and ″AUUAAUUGUAAAAGGUAAAC″ as SEQ ID NO: 8.
1. 9 ul UM DNA + 1 ul Cas9/gRNA COVID 28112 C AUUAAUUGUAAAAGGUGAAC
2. 9 ul UM DNA + 1 ul Cas9/gRNA COVID 28112 T AUUAAUUGUAAAAGGUAAAC
3. 9 ul UM DNA + 1 ul Cas9
4. 2.5 ul cDNA + 1 ul Cas9/gRNA COVID 28112 C AUUAAUUGUAAAAGGUGAAC + 6.5 ul
Dilution buffer
5. 2.5 ul cDNA + 1 ul Cas9/gRNA COVID 28112 T AUUAAUUGUAAAAGGUAAAC + 6.5 ul
Dilution buffer
6. 2.5 ul cDNA + 1 ul Cas9 + 6.5 ul Dilution buffer
7. Positive control:. 3.5 ul DNA + 1 ul Cas9/gRNA COVID 28112 C AUUAAUUGUAAAAGGUGAAC +
5.5 ul Dilution buffer
8. Positive control:. 3.5 ul DNA + 1 ul Cas9/gRNA COVID 28112 7 AUUAAUUGUAAAAGGUAAAC +
5.5 ul Dilution buffer
9. 3.5 ul DNA + 1 ul Cas9 + 6.5 ul Dilution buffer

### Example 6

This Example describes an experiment intended to determine whether labeling and detection is gRNA sequence-dependent.

An irrelevant gRNA for the human NR3C1 gene (that is, a sequence with no relation to SARS-CoV-2) termed BE2 GR was used to interrogate a SARS-CoV-2 DNA sequence. This assay was performed in the background of competitor mCherry plasmid DNA containing a 250 nucleotide GGG and a 911 nucleotide GG sequences. It was expected that biotinylated SpCas9 would bind the GGG or NGG PAM sequences in mCherry as part of its scanning function, preventing off-target/non-specific binding.

Amplification and detection with PCR or LFA were performed as described in Examples 4 and 5 with the following modifications. Different concentrations of target DNA and mCherry (at a 3-10 fold molar excess of mCherry plasmid) were added (as described in Table 5B) when the target DNA was mixed with biotinylated Cas9. Additionally, in conditions 1-3, an irrelevant gRNA, BE2 GR, was added instead of a COVID gRNA. The sequence of BE2 GR is described in Table 5A.

Results are shown in FIG. 7. Unexpectedly, non-specific binding was observed with the irrelevant gRNA; competitor DNA (mCherry) at the concentrations employed inhibited but did not eliminate off-target/non-specific binding (see Conditions 1-3).

TABLE 5A
Irrelevant (i.e., non-COVID-19) gRNA control

| gRNA | Sequence | SEQ ID NO: |
|---|---|---|
| BE2 GR | UCAGAAcAGCAACAUUUGAA | 22 |

TABLE 5B
Table 5B discloses ″AUUAAUUGUAAAAGGUGAAC″ as SEQ ID NO: 7
and ″AUUAAUUGUAAAAGGUAAAC″ as SEQ ID NO: 8.
1.5 ul of cDNA = 1582 nM
3 ul of BE gRNA with 10 ul of Cas
1. 1.5 ul cDNA + 1 ul Cas9/gRNA BE2 GR + 7.5 ul Dilution buffer
2. 1.5 ul cDNA + 1 ul Cas9/gRNA BE2 GR + 7.5 ul mCherry plasmid
3. 1.5 ul cDNA + 1 ul Cas9/gRNA BE2 GR + 15 UL mCherry plasmid
4. 1.5 ul cDNA + 1 ul Cas9/gRNA COVID 28112 C AUUAAUUGUAAAAGGUGAAC + 7.5 ul MCHerry
plasmid DNA
5. 1.5 ul cDNA + 1 ul Cas9/gRNA COVID 28112 C AUUAAUUGUAAAAGGUGAAC + 15 ul mCHerry
plasmid DNA
6. 1.5 ul cDNA + 1 ul Cas9/gRNA COVID 28112 T AUUAAUUGUAAAAGGUAAAC + 7.5 ul mCHerry
plasmid DNA
7. 1.5 ul cDMA + 1 ul Cas9/gRNA COVID 28112 T AUUAAUUGUAAAAGGUAAAC + 15 ul mCHerry
plasmid DNA
8. Positive control:. 1.5 ul DNA + 1 ul Cas9/gRNA COVID 28112 T AUUAAUUGUAAAAGGUAAAC +
7.5 ul Dilution buffer
9. 1.5 ul DNA + 1 ul Cas9 + 6.5 ul Dilution buffer

### Example 7

This Example describes an experiment intended to test whether increasing the amount of competitor plasmid DNA could prevent non sequence-dependent detection.

Amplification and detection with LFA were performed as described in Example 6 expect that increasing amounts of mCherry (>10 fold molar excess) were used, a described in Table 6.

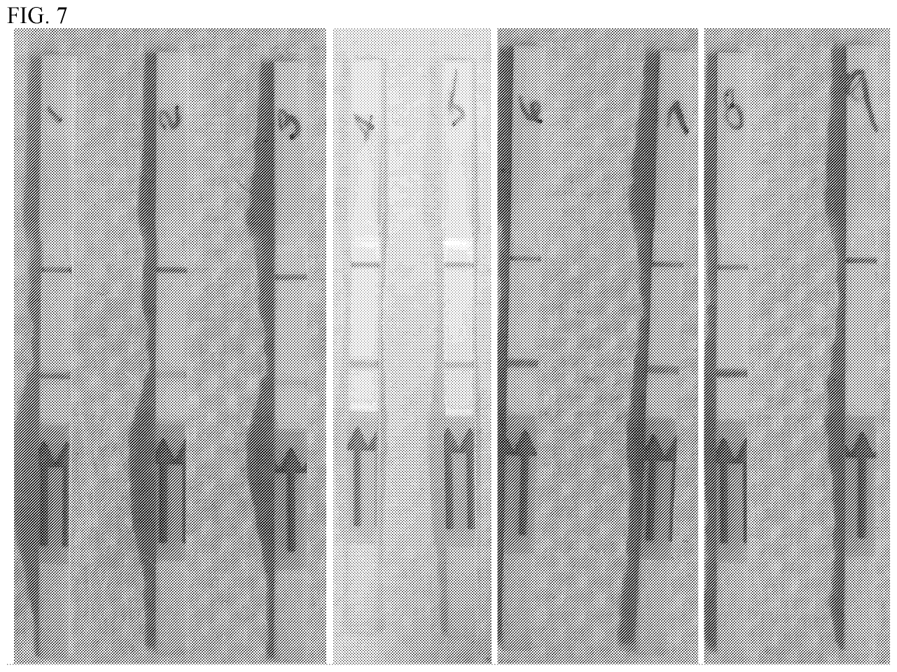

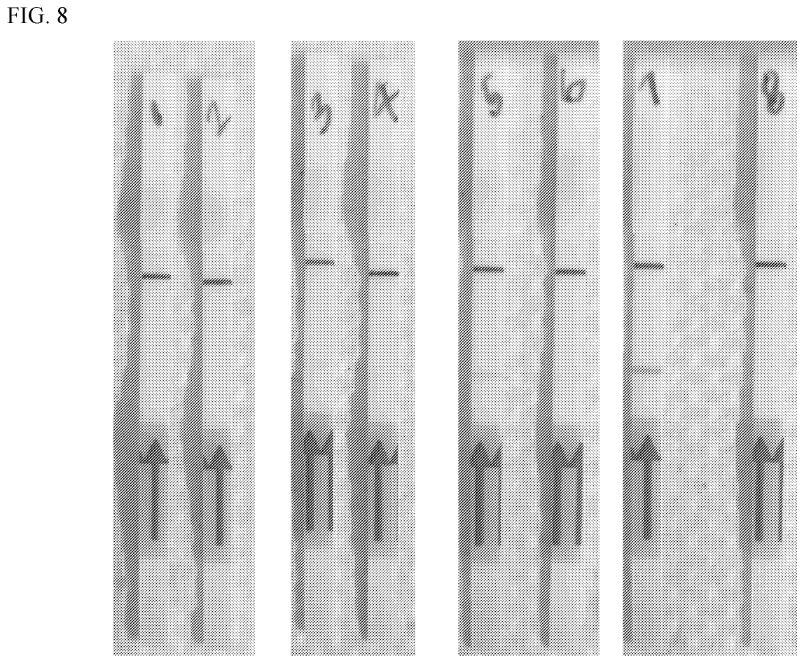

Results are shown in FIG. 8. Competitor DNA eliminated non-specific binding (Conditions 1 and 2) and allowed for COVID sequence detection (Conditions 3-6).

TABLE 6
Table 6 discloses ″AUUAAUUGUAAAAGGUGAAC″ as SEQ ID NO: 7
and ″AUUAAUUGUAAAAGGUAAAC″ as SEQ ID NO: 8.
1.5 ul of cDNA = 1582 nM
3 ul of BE gRNA with 10 ul of Cas
DNA is pooled and is at 0.5 ng/ul, Cas and gRNA frozen at -20 for 1 h
1. 2 ul DNA + 1 ul Cas9/gRNA BE2 GR + 30 ul mCherry plasmid DNA
2. 2 ul DNA + 1 ul Cas9/gRNA BE2 GR + 60 ul mCherry plasmid DNA
3. 2 ul DNA + 1 ul Cas9/gRNA COVID 28112 C AUUAAUUGUAAAAGGUGAAC + 30 ul mCherry
plasmid DNA
4. 2 ul DNA + 1 ul Cas9/gRNA COVID 28112 C AUUAAUUGUAAAAGGUGAAC + 60 ul mCherry
plasmid DNA
5. 2 ul DNA + 1 ul Cas9/gRNA COVID 28112 T AUUAAUUGUAAAAGGUAAAC + 30 ul mCherry
plasmid DNA
6. 2 ul DNA + 1 ul Cas9/gRNA COVID 28112 T AUUAAUUGUAAAAGGUAAAC + 60 ul mCherry
plasmid DNA
7. Positive control:. 2 ul DNA + 1 ul Cas9/gRNA COVID 28112 T AUUAAUUGUAAAAGGUAAAC +
60 ul Dilution buffer
8. 2 ul DNA DNA + 60 ul Dilution buffer

### Example 8

In the previous examples, the Ultramer had been amplified using PCR. This Example describes an experiment that tested if a SARS-CoV-2 sequence amplified by RPA with a FAM-labeled forward primer and an unlabeled reverse primer could be detected with LFA.

RPA was performed according to package instructions (TwistAmp® Basic; TABAS03KIT, TwistDx, Cambridge, United Kingdom) using the following components:

10 uM FAM RT 28112 F: 12 ul

10 uM Unlabeled REV: 12 ul

Hybridization soln: 147.5

HOH: 61

At the same time, Cas9 was complexed with gRNA, as follows:

Cas9 set up. Resuspend Cas9 in 30 ul and add 1 ul of 28112 gRNA or 3 ul of Gr2 gRNA

25 ul of mCherry plasmid (953 ng/ul)

73 ul dilution buffer

2 ul of Cas9:gRNA

Each incubated at 37 C×30 minutes.

5 μL of the RPA product was added to 20 μL of the Cas9:gRNA complex, and the combination was incubated to 30 minutes at 37° C.

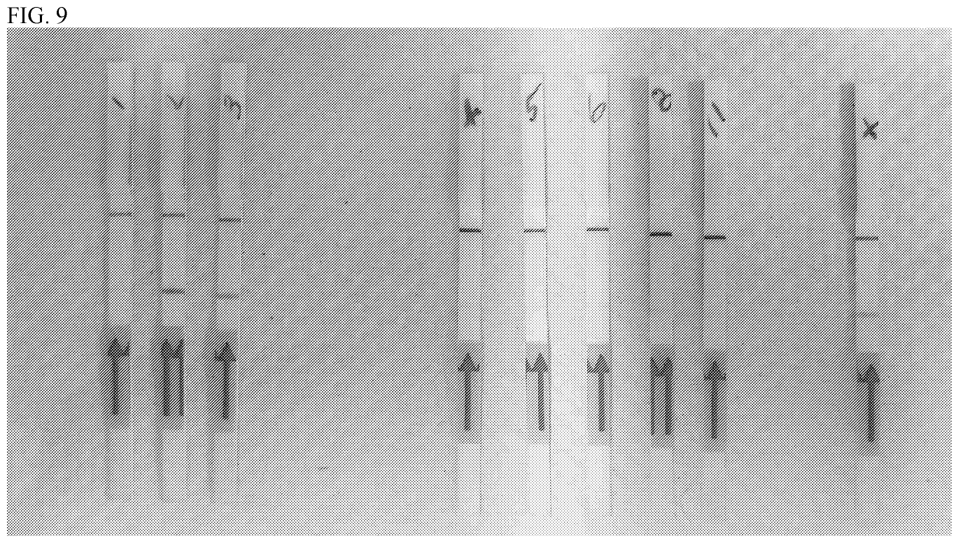

LFA was performed as described in Example 6. Results are shown in FIG. 9.

The results of conditions 1-3 indicate that non-specific binding of the sgRNA:Cas9 complex did not occur (condition 1) but COVID-19 could be detected (conditions 2 and 3).

TABLE 7
(HOH = water)
Table 7 discloses ″AUUAAUUGUAAAAGGUGAAC″ as SEQ ID NO: 7 and
″AUUAAUUGUAAAAGGUAAAC″ as SEQ ID NO: 8.
1. UM + gDNA + BE2 gRNA
2. UM + gDNA + Cas9/gRNA COVID 28112 T AUUAAUUGUAAAAGGUAAAC
3. UM + gDNA + COVID 28112 C AUUAAUUGUAAAAGGUGAAC
4. Jurkat gDNA + Cas9/BE2 gRNA
S. Jurkat gDNA Cas9/gRNA COVID 28112 T AUUAAUUGUAAAAGGUAAAC
6. Jurkat gDNA + COVID 28112 C AUUAAUUGUAAAAGGUGAAC
7. NTC + Primers + Cas9/BE2 gRNA
8. NTC + Primers Cas9/gRNA COVID 28112 T AUUAAUUGUAAAAGGUAAAC
9. NTC + Primers + COVID 28112 C AUUAAUUGUAAAAGGUGAAC
10. HOH + Cas9/BE2 gRNA
11. HOH + Cas9/gRNA COVID 28112 T AUUAAUUGUAAAAGGUAAAC
12. HOH + COVID 28112 C AUUAAUUGUAAAAGGUGAAG
13. + Control (amplicon of 28112 + T gRNA + Cas)

### Example 9

This Example describes testing to determine if a SARS-CoV-2 sequence amplified by PCR with a FAM-labeled forward primer and an unlabeled reverse primer could be detected in the presence of a molar excess of a small double stranded oligo nucleotide sequence (see Table 8a) as the competitor DNA instead of mCherry, as described in Example 7.

TABLE 8A
Competitor oligonucleotide

| DNA Sequence | | SEQ ID |
|---|---|---|
| (5′-3′) | Sequence | NO: |
| Soak Top | CGGGAGGGTGGGCGGGAGGGTGGGCGGGAGGGTGGGCGGGA | 23 |
| | GGGTGGG | |
| Soak Bottom | CCCACCCTCCCGCCCACCCTCCCGCCCACCCTCCCGCCCACCC | 24 |
| | TCCCG | |

TABLE 8B

| gRNA | Sequence | SEQ ID NO: |
|---|---|---|
| COVID 28112 T | AUUAAUUGUAAAAGGUAAAC | 8 |
| BE2 | UCAGAAcAGCAACAUUUGAA | 22 |

TABLE 8C

| | Fold Excess of | | Dilution |
|---|---|---|---|
| Condition | Competitor DNA | Components | Buffer |
| 1 | 10:1 | 0.1 μL soak oligo + 1 μL of DNA + 1 μL Cas9/gRNA COVID 28112 T | 20 μL |
| 2 | 10:1 | 0.1 μL soak oligo + 1 μL of DNA + 1 μL Cas9/gRNA Cas9/BE2 gRNA | 20 μL |
| 3 | 100:1 | 1 μL soak oligo + 1 μL of DNA + 1 μL Cas9/gRNA COVID 28112 T | 19 μ |
| 4 | 100:1 | 1 μL soak oligo + 1 μL of DNA + 1 μL Cas9/gRNA Cas9/BE2 gRNA | 19 μ |
| 5 | 1000:1 | 10 μL soak oligo + 1 μL of DNA + 1 μL Cas9/gRNA COVID 28112 T | 10 μL |
| 6 | 1000:1 | 10 μL soak oligo + 1 μL of DNA + 1 μL Cas9/gRNA Cas9/BE2 gRNA | 10 μL |
| 7 | 2,000:1 | 20 μL soak oligo + 1 μL of DNA + 1 μL Cas9/gRNA COVID 28112 T | n/a |
| 8 | 2,000:1 | 20 μL soak oligo + 1 μL of DNA + 1 μL Cas9/gRNA Cas9/BE2 gRNA | n/a |

Endpoint PCR was done as described in Example 2, and a molar excess of the competitor (‘soak oligo’) DNA (see Table 8A) of 10-2000 fold (relative to the target oligonucleotide) was included.

Results are shown in FIG. 10.

The results of conditions 1-4 indicate that non-specific binding of the sgRNA:Cas9 complex did occur, as indicated by the lines indicated by the arrow in conditions 2 and 4, but non-specific binding of the sgRNA:Cas9 complex did not occur in conditions 6 and 8 allowing for unambiguous detection of COVID-19 at those dilutions when COVID-19 gRNA present (conditions 5 and 7).

### Example 10

To diminish non-specific binding and to promote single base pair/SNP detection (for example, distinguishing between gRNAs differ from one another by 1 base pair (bp) in the targeting portion), binding salt (NaCl) concentrations of 0 to 5 M will be tested.

### Example 11

To further promote SNP detection/single base pair resolution, the effect of including ‘soak oligos’ (when the gRNA is exposed to the target nucleotide) will be tested, the soak oligos having the sequences shown in Table 9A. Exemplary test conditions are shown in Table 9B.

TABLE 9A
Perfectly complementary to: COVID 28112 C gRNA: aUUaaUUgUaaaaggUGaac (SEQ ID NO: 7)
C Soak Forward (5′-3′): GGAGGGTGGGGATTAATTGTAAAAGGTGAACGGGCGGGAGGGTGG (SEQ ID
NO: 25)
C Soak Reverse: CCACCCTCCCGCCCGTTCACCTTTTACAATTAATCCCCACCCTCC (SEQ ID NO: 26)
Perfectly complementary to: COVID 28112 T gRNA: aUUaaUUgUaaaaggUAaac (SEQ ID NO: 8)
T Soak Forward: GGAGGGTGGGGATTAATTGTAAAAGGTAAACGGGCGGGAGGGTGG (SEQ ID NO: 27)
T Soak Reverse: CCACCCTCCCGCCCGTTTACCTTTTACAATTAATCCCCACCCTCC (SEQ ID NO: 28)

TABLE 9B
1. T soak oligo (with 1 bp mismatch to gRNA) + C target nucleotide
(perfect match to gRNA) + Cas9/ COVID 28112C gRNA
2. C soak oligo (with 1 bp mismatch to gRNA) + T target nucleotide
(1 bp mismatch to gRNA) + Cas9/ COVID 28112C gRNA
3. non-homologous soak oligo + T target nucleotide (with 1 bp mismatch
to gRNA) + Cas9/ COVID 28112C gRNA
4. non-homologous soak oligo + T target nucleotide (perfect match to
gRNA) + Cas9/ COVID 28112T gRNA

By using a gRNA that is perfectly complimentary to a target nucleotide sequence (for example, COVID 28112 C) in combination with competing DNA with a mutation (for example, soak oligos of the T variety (T Soak forward/reverse)), it is expected that the perfectly complimentary gRNA will bind to the target nucleotide (for example, the COVID 28112 C sequence) at single base pair resolution.

Without wishing to be bound by theory, it is believed that the competing (“soak”) DNA which is unlabeled and has a 1 bp mismatch to the gRNA will compete with the target nucleotide (which is labeled) for binding to the Cas9 and gRNA. When the Cas9 and gRNA are perfectly complimentary to the (labeled) target nucleotide, and when the target nucleotide is bound in a Cas9-gRNA complex, the complex may be detected via LFA. In contrast, when the soak DNA is bound, the complex will not be detected. However, as shown in Example 4, when the Cas9 and gRNA are not perfectly complimentary to the (labeled) target nucleotide, they still bind to the target nucleotide likely because Cas9 exhibits transient DNA binding whilst scanning for the requisite PAM and perfect complementarity.

The following results to exemplary test conditions shown in Table 9B are expected:

1. positive test band on LFA.
2. negative test band on LFA (because binding otherwise seen between target nucleotide having 1 bp mismatch from gRNA and gRNA-Cas9 is interrupted by the gRNA-Cas9 binding to the perfectly matched soak).
3. positive test band on LFA (as shown in the previous Examples, although a non-homologous soak can prevent binding of a target nucleotide having limited/no homology with the gRNA, binding is observed between target nucleotide having 1 bp mismatch from the gRNA and gRNA-Cas9).
4. positive test band on LFA (positive control).

TABLE 10

| 3 | Primer | SEQ | SEQ ID NO: |
|---|---|---|---|
| 5 | 8750 Dig UM F | /5DigN/gtcactcgtgacatagcatctacag | 29 |
| 6 | 8750 UM R | ttgtgcgtaatatcgtgccagg | 30 |
| 7 | 8750 UM R bio | 5Biosg/ttgtgcgtaatatcgtgccagg | 31 |
| 9 | 8750 T > C DNA UM | gtcactcgtgacatagcatctacagatacttgtttt | 13 |
| | | gctaacaaacatgctgatttt gacacatggtttagCcagcg tgg | |
| | | tggtagttatact | |
| | | aatgacaaagcttgcccattgattgctgcagtcataacaagagaagtgg | |
| | | gttttgtcgtgcctggtttgcctggcacgatattacgcacaa | |
| 11 | 8750 T>C RNA UM | taacaaacatgctgattttgacacatgg tttagCcagcg tgg | 32 |
| | | tggtagttatact aatgacaaagcttgcccattgattgctgcagtcata | |
| | | acaagagaa gtgggttttgtcgtgcctgg | |
| 12 | RT 8750 RNA RT F | taacaaacatgctgattttgacacatgg | 1 |
| 13 | RT 8750 RNA Dig RT | /5DigN/taacaaacatgctgattttgacacatgg | 17 |
| | F | | |
| 14 | RT 8750 RNA RT REV | ccaggcacgacaaaacccac | 2 |
| 15 | RT 8750 RNA BIO RT | 5Biosg/ccaggcacgacaaaacccac | 18 |
| | REV | | |
| 17 | 8750 T gRNA | TAATACGACTCACTATAGG | 33 |
| | | gacacatggtttagTcagcg GTTT T | |
| | | AGAGCTAGAAATAGCAAGTTA | |
| | | AAATAAGGCTAGTCCGTTATCAACTTGAAA | |
| | | AAGTGGCACCGAGTCGGTGC TTTT TTT | |
| 19 | 8750 C gRNA | TAATACGACTCACTATAGG | 34 |
| | | gacacatggtttagCcagcg GTTT T | |
| | | AGAGCTAGAAATAGCAAGTTA | |
| | | AAATAAGGCTAGTCCGTTATCAACTTGAAA | |
| | | AA GTGGCACCGAGTCGGTGC TTTT TTT | |
| 21 | T7 F | TAATACGACTCACTATAGG | 35 |
| 22 | PT REV | caacacgaac gtcatgatac tc | 36 |
| 24 | 28112 FAM UM F | /56-FAM/Gaattgtgcgtggatgaggctgg | 21 |
| 25 | 28112 UM REV | caacacgaacgtcatgatactc | 6 |
| 26 | 28112 UMM BIO REV | /5Biosg/caacacgaacgtcatgatactc | 37 |
| 28 | 28112 UM | Gaattgtgcgtggatgaggctggttctaaatcacccattcagtacatcg | 15 |
| | | atatcggtaattatacagtttcctgttTaccttttacaattaattg | |
| | | ccaggaacctaaattgggtagtcttgtagtgcgttgttcgttctatgaaga | |
| | | ctttttagagtatcatgacgttcgtgttg | |
| 30 | 28112 C > T RNA UM | ctaaatcacccattcagtacatcgatatcggtaattatacagtttcctgt | 38 |
| | | tTaccttttacaattaattgccaggaacctaaattgggtagtcttgtag | |
| | | tgcgttgttcgttctatgaag | |
| 32 | RT 28112 RNA RT F | ctaaatcacccattcagtacatcgatatcg | 3 |
| 34 | RT 28112 RNA RT FAM | /56-FAM/ctaaatcacccattcagtacatcgatatcg | 19 |
| | F | | |
| 36 | RT 28112 RNA RT REV | cttcatagaacgaacaacgcactacaagactacc | 4 |
| 38 | RT 28112 RNA bio RT | 5Biosg/cttcatagaacgaacaacgcactacaagactacc | 20 |
| | REV | | |
| 39 | RT 28112 RNA DIG RT | /5DigN/cttcatagaacgaacaacgcactacaagactacc | 39 |
| | REV | | |
| 40 | 8750 C gRNA | TAATACGACTCACTATAGG | 40 |
| | | attaattgtaaaaggtGaac GTTT T | |
| | | AGAGCTAGAAATAGCAA | |
| | | GTTAAAATAAGGCTAGTCCGTTATCAACTT | |
| | | GAAAAAGTGGCACCGAGTCGGTGC TTTT | |
| | | TTT | |
| 42 | 8750 T gRNA | TAATACGACTCACTATAGG | 41 |
| | | attaattgtaaaaggtAaac GTTT T | |
| | | AGAGCTAGAAATAGCAA | |
| | | GTTAAAATAAGGCTAGTCCGTTATCAACTT | |
| | | GAAAAA GTGGCACCGAGTCGGTGC TTTT | |
| | | TTT | |
| 44 | 8750 T7 for making | TAATACGACTCACTATAGG | 42 |
| | RNA of the target | taacaaacatgctgattttgacacatgg | |
| 46 | 8750 T7 REV | ccaggcacgacaaaacccac | 2 |
| 48 | b actin RT F | ATATCGCCGCGCTCGTCGTC | 43 |
| 50 | b actin RT REV | ATGTCGTCCCAGTTGGTGACG | 44 |
| 52 | b actin bio REV | 5Biosg/ATGTCGTCCCAGTTGGTGACG | 45 |
| 54 | b actin DIG F | /5DIGN/ATATCGCCGCGCTCGTCGTC | 46 |
| 56 | b actin FAM F | /56-FAM/ATATCGCCGCGCTCGTCGTC | 47 |
| 58 | b actin DIG REV | /5DIGN/ATGTCGTCCCAGTIGGTGACG | 48 |
| 61 | 8750 T7 for FL UM | TAATACGACTCACTATAGG | 49 |
| | (cell 9) | gtcactcgtgacatagcatctacag | |
| 63 | 8750 TREV for RNA | ttgtgcgtaatatcgtgccagg | 30 |
| | for FL UM (cell 9) | | |
| 65 | 28112 T7 for FL UM | TAATACGACTCACTATAGGGaattgtgcgtggatgag | 50 |
| | (cell 28) | gctgg | |
| 67 | 28112 T7 REV for FL | caacacgaacgtcatgatactc | 6 |
| | UM (cell 28) | | |

### Example 12

### Nucleic Acids

Oligonucleotide and sgRNAs are detailed by sequence and function in Tables 11-14. All primers and synthetic fragments were produced by Integrated DNA Technologies (IDT), Coralville, Iowa, and are shown 5′-3′.

TABLE 11
Primers and synthetic templates.

| | | | SEQ ID |
|---|---|---|---|
| Identification | Sequence (5′-3′) | Function | NO: |
| SARS-Co-V2 | /56- | FAM/FITC-labeled forward | 21 |
| FAM Forward | FAM/GAATTGTGCGTGGATGAGGCTGG | primer for SARS-Co-V2 | |
| | | amplification from synthetic or | |
| | | viral genome templates. | |
| SARS-Co-V2 | /5Biosg/CAACACGAACGTCATGA | Biotinylated-labeled reverse | 37 |
| Biotinylated Reverse | TACTC | primer for SARS-Co-V2 | |
| | | amplification from synthetic | |
| | | template. | |
| SARS-Co-V2 | CAACACGAACGTCATGATACTC | Reverse primer for SARS-Co-V2 | 6 |
| Unlabeled Reverse | | amplification from synthetic | |
| | | template. | |
| SARS-Co-V2 | TTAGATGAAATCTAAAACAACACG | Reverse primer for SARS-Co-V2 | 55 |
| Genome Reverse | | amplification from genomic RNA | |
| | | template. | |
| SARS-Co-V2 T SNP | GAATTGTGCGTGGATGAG | Synthetic SARS-COV-2 PCR | 28 |
| DNA PCR template. | GCTGGTTCTAAATCACCCA | template containing a thymine | |
| | TTCAGTACATCGATATCGGT | SNP at position 28144. | |
| | AATTATACAGTTTCCTGTTTA | | |
| | CCTTTTACAATTAATTGCCAG | | |
| | GAACCTAAATTGGGTAGTCTT | | |
| | GTAGTGCGTTGTTCGTTCTATG | | |
| | AAGACTTTTTAGAGTATCATG | | |
| | ACGTTCGTGTTG | | |
| SARS-Co-V2 C SNP | GAATTGTGCG TGGATGAG | Synthetic SARS-COV-2 PCR | 56 |
| DNA PCR template. | GC TGGTTCTAAA TCACCC | template containing a cytosine | |
| | ATTC AGTACATCGA TATC | SNP at position 28144. | |
| | GGTAAT TATACAGTT TCC | | |
| | TGTTCAC CTTTTACAAT TA | | |
| | ATTGCCAG GAACCTAAAT | | |
| | TGGGTAGTCT TGTAGTGCG | | |
| | T TGTTCGTTCT ATGAAGACT | | |
| | T TTTAGAGTAT CATGACGTTC | | |
| | GTGTTG | | |
| Influenza A Forward | CAAGACCAATCYTGTCACCTCTGAC | Forward primer for Influenza A | 57 |
| | | amplification from synthetic | |
| | | DNA. | |
| Influenza A Reverse | GCATTYTGGACAAAVCGTCTACG | Reverse primer for Influenza A | 58 |
| | | amplification from synthetic | |
| | | DNA. | |
| Synthetic Influenza A | GCTCTCATGGAATGGCTAAAGACA | Synthetic PCR template to | 59 |
| DNA PCR template | AGACCAATCTTGTCACCTCTGACTA | generate Influenza A DNA | |
| | AGGGGATTTTAGGATTTGTGTTCAC | amplicons | |
| | GCTCACCGTGCCCAGTGAGCGAGG | | |
| | ACTGCAGCGTAGACGCTTTGTCCAA | | |
| | AATGCCCTAAATGGGAATGGGGAC | | |
| | CCGAACAACATGG | | |
| Influenza B Forward | TCCTCAAYTCACTCTTCGAGCG | Forward primer for Influenza B | 60 |
| | | amplification from synthetic | |
| | | DNA. | |
| Influenza B Reverse | CGGTGCTCTTGACCAAATTGG | Reverse primer for Influenza B | 61 |
| | | amplification from synthetic | |
| | | DNA. | |
| Synthetic Influenza | TACAGTGGAG GATGAAGAAG ATGG | Synthetic PCR template to | 62 |
| B DNA PCR template | CCATCG GATCCTCAAT TCACTCTTC | generate Influenza B DNA | |
| | G AGCGTCTTAA TGAAGGACAT TCA | amplicons. | |
| | AAGCCAA TTCGAGCAGC TGAAACT | | |
| | GCG GTGGGAGTCT TATCCCAATT TG | | |
| | GTCAAGAG CACCGACTAT CACCAG | | |
| | AAGA GGGAGACAAT | | |
| RSV Forward | GGCAAATATG GAAACATACG TGAA | Forward primer for RSV | 63 |
| | | amplification from synthetic | |
| | | DNA. | |
| RSV Reverse | CATGGGCACC CATATTGTAA | Reverse primer for RSV from | 64 |
| | | synthetic DNA. | |
| Synthetic RSV DNA | GGCAAATATG GAAACATACG TG | Synthetic PCR template to | 65 |
| PCR template | AACAAGCT TCACGAAGGC TCCA | generate RSV DNA amplicons. | |
| | CATACA CAGCTGCTGT TCAATAC | | |
| | AAT GTCCTAGAAA AAGACGATG | | |
| | A CCCTGCATCA CTTACAATAT GG | | |
| | GTGCCCAT GTTCCAATCA | | |

FAM = 6-Carboxyfluorescein;
5Biosg = 5′biotin;
SNP = single nucleotide polymorphism.

### Amplification

DNA template fragments were amplified with the indicated forward and reverse primers.

Endpoint PCR was performed using 1 μM template and 0.2 μM final concentration of primers in a 100 μL reaction volume with Phusion High-Fidelity PCR Master Mix (Thermo Fisher, Waltham, Mass.) under the following conditions: 98° C.×2 min and 34 cycles of 98° C.×10 s, 62° C.×10 s, and 72° C.×15 s, with a final extension of 5 min.

Recombinase polymerase amplification (RPA): 1 μM of template was used with primers for recombinase polymerase amplification according to the manufacturer's instructions for the TwistDx Basic RPA Kit from TwistDx (Maidenhead, UK).

Fluorescent Probes: Probes were purchased from IDT (Coralville, Iowa, USA) and resuspended at 100 μM and contain Iowa Black quencher (3IABkFQ or 3IAbRQSp). The 5′ fluorescent labels are: FAM=6-Carboxyfluorescein, TexRd=Texas Red, YakYel=Yakima Yellow, Cy3=Cyanine 3, and TAMRA=5-Carboxytetramethylrhodamine.

TABLE 12
Fluorescent assay nucleic acids.

| Identification | Sequence (5′-3′) | Function SEQ ID NO: |
|---|---|---|
| COVID T Probe: | /56-FAM/TCCTGTTTACCTTTTA | SARS-COV-2 with a |
| | CAATTAATTGCCAGGA/3IABKFQ/ | FAM fluorescent probe 66 |
| | | with Iowa Black |
| | | quencher that recognizes |
| | | thymine at position |
| | | 28144. |
| COVID C Probe: | /56-FAM/TCCTGTTCACCTTTTA | FAM fluorescent probe 67 |
| | CAATTAATTGCCAGGA/3IABKFQ | with Iowa Black |
| | | quencher that recognizes |
| | | SARS-COV-2 with a |
| | | cytosine at position |
| | | 28144. |
| Influenza A Probe: | GGGCACGGTGAGCGTGA/3IAbRQ | Texas Red fluorescent 68 |
| | /5TexRd-XN/CAGTCCTCGCTCACT | probe with Iowa Black |
| | Sp/ | quencher that recognizes |
| | | Influenza A sequence. |
| Influenza B Probe: | /5YAKYel/TCCCACCGCAGTTTCAG | Yakima Yellow 69 |
| | CTGCTCGAATTGGCT/3IABKFQ/ | fluorescent probe with |
| | | Iowa Black quencher that |
| | | recognizes Influenza B |
| | | sequence. |
| RSV Probe: | /5Cy3/GCTCCACATACACAGCTG | Cy3 fluorescent probe 70 |
| | CTGTTCAATACAAT/3IAbRQSp/ | with Iowa Black |
| | | quencher that recognizes |
| | | RSV sequence. |
| RSV Probe: | /56- | TAMRA fluorescent 71 |
| | TAMN/GCTCCACATACACAGCTG | probe with Iowa Black |
| | CTGTTCAATACAAT/3IAbRQSp/ | quencher that recognizes |
| | | RSV sequence. |

### Soak DNA Oligonucleotides

The oligonucleotides were purchased from IDT (Coralville, Iowa, USA) and resuspended at 200 μM. Equal molar equivalents were mixed in Tris NaCl and denatured and renatured by heating for 5 min at 95° C. and cooling to room temperature at a rate of −0.1° C./s.

TABLE 13
Lateral flow assay soak oligonucleotides.

| Identification | Sequence (5′-3′) | Function | SEQ ID NO: |
|---|---|---|---|
| PAM Soak | CGGGAGGGTGGGCGGGAGGGTGGG | PAM-rich ODN that acts | 23 |
| Forward: | CGGGAGGGTGGGCGGGAGGGTGGG | as a bait for promiscuous | |
| | | Cas9 binding; | |
| | | sense/top/forward/strand. | |
| PAM Soak | CCCACCCTCCCGCCCACCCTCCCG | PAM-rich ODN that acts | 24 |
| Reverse: | CCCACCCTCCCGCCCACCCTCCCG | as a bait for promiscuous | |
| | | Cas9 binding; anti- | |
| | | sense/bottom/reverse/ | |
| | | strand. | |
| C Soak | GGAGGGTGGGGATTAATTGTAAA | Bait ODN that contains | 25 |
| Forward: | AGGTGAACGGGCGGGAGGGTGG | SARS-COV-2 sequences | |
| | | and a cytosine at position | |
| | | 28144; | |
| | | sense/top/forward/strand. | |
| | | In yellow is the anti- | |
| | | parralel guanine. | |
| C Soak | CCACCCTCCCGCCCGTTCACCTTT | Bait ODN that contains | 26 |
| Reverse: | TACAATTAATCCCCACCCTCC | SARS-COV-2 sequences | |
| | | and a cytosine at position | |
| | | 28144; anti- | |
| | | sense/bottom/reverse/ | |
| | | strand. | |
| T Soak | GGAGGGTGGGGATTAATTGTAAA | Bait ODN that contains | 27 |
| Forward: | AGGTAAACGGGCGGGAGGGTGG | SARS-COV-2 sequences | |
| | | and a thymine at position | |
| | | 28144; | |
| | | sense/top/forward/strand. | |
| | | In yellow is the anti- | |
| | | parralel adenine. | |
| T Soak | CCACCCTCCCGCCCGTTTACCTTT | Bait ODN that contains | 28 |
| Reverse: | TACAATTAATCCCCACCCTCC | SARS-COV-2 sequences | |
| | | and a thymine at position | |
| | | 28144; anti- | |
| | | sense/bottom/reverse/ | |
| | | strand. | |

### Guide RNAs

All single guide RNAs (sgRNA) were purchased from Synthego (Menlo Park, Calif., USA) and resuspended at 100 μM in Tris Ethylenediaminetetraacetic acid. Shown are the 20 bp sequences specific to the corresponding gene target. The remaining sgRNA architecture is the vendor supplied standard sequence for Streptococcus pyogenes Cas9 binding.

TABLE 14
single guide RNA sequences

| Identifi- | Sequence | | SEQ I [symbol] |
|---|---|---|---|
| cation. | (5′-3′) | Function | NO: |
| COVID-19 T | AUUAAUUGUA | Recognizes ORF8a that has a | 8 |
| sgRNA: | AAAGGUAAAC | thymine at nucelotide position | |
| | | 28144. | |
| COVID-19 C | AUUAAUUGUA | Recognizes ORF8a that has a | 7 |
| sgRNA: | AAAGGUGAAC | cytosine at nucelotide position | |
| | | 28144. | |
| Influenza A | CUCACCGUGC | Recognizes the influenza A | 51 |
| sgRNA: | CCAGUGAGCG | amplicon. | |
| Influenza B | AAUUCGAGCA | Recognizes the influenza B | 52 |
| sgRNA: | GCUGAAACUG | amplicon. | |
| RSV sgRNA: | UUGAACAGCA | Recognizes the influenza RSV | 53 |
| | GCUGUGUAUG | amplicon. | |
| Control | CACUGGCUGU | Irrelevant control sgRNA that has | 54 |
| sgRNA: | CGCUUCUCAA | no homology to viral genomes. | |

[symbol]

indicates data missing or illegible when filed

### Biotinylated Cas9 and Lateral Flow

dCas9-3×FLAG™-Biotin Protein containing the D10A and H840A mutations from Milipore Sigma (Merck KGaA, Darmstadt, Germany) was resuspended in the included Reconstitution Solution to ˜1.7 mg/mL (8 pmol/μL), and 1 μL of 100 μM sgRNA was added to give ˜1.2 M excess of sgRNA. Cas9:sgRNA complexing was allowed to occur at room temperature for five minutes.

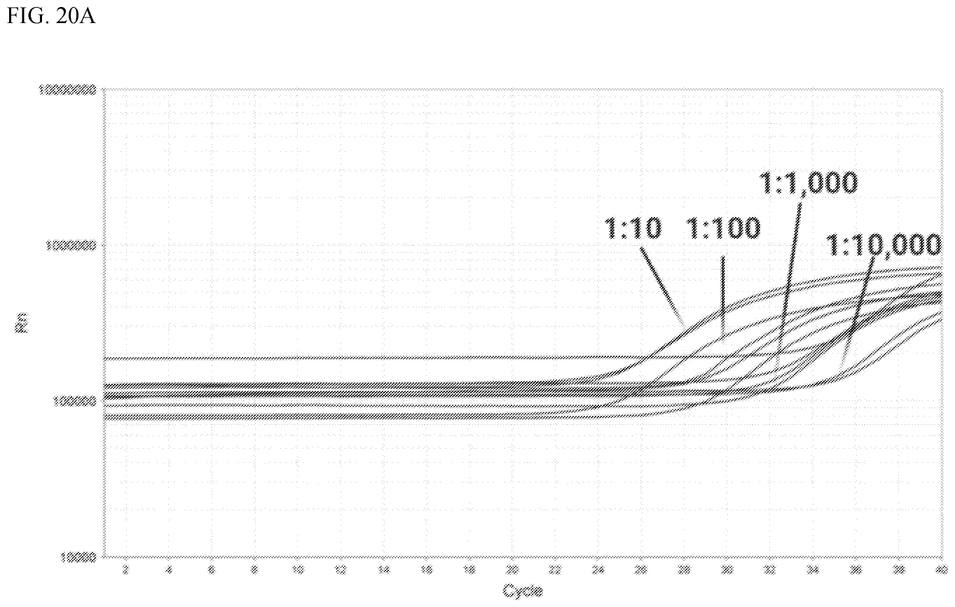

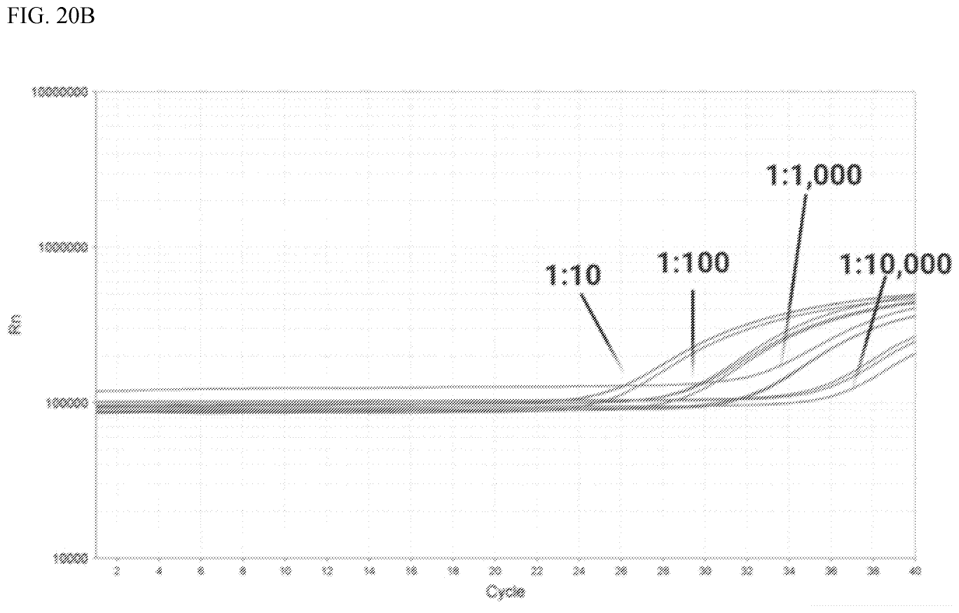

10 μL of unpurified PCR or RPA products was used with the Cas9:sgRNA complex in a 50 reaction at 37° C. Where indicated, the double-stranded soak DNAs were used at the concentrations shown in the relevant figures. 20 μL of the Cas9 reaction was used for detection using the TwistDx Milenia HybriDetect1 lateral flow assay (Maidenhead, UK) under the manufacturer's recommendation.

For the assays in which Cas9 was included in the RPA, the reaction conditions and primer concentrations remained as above. Instead of using water to achieve the final 50 μL reaction volume, the indicated concentrations of soak ODNs were used to reach the final volume. RPA then proceeded at 37° C. with use of 20 μL of the post-reaction product for LFA.

### CRISPR/Cas9 Fluorescence Assay

Equal volumes of amplification product and 100 μM disease-specific probe were mixed and denatured/renatured by heating for 5 min at 95° C. and cooling to room temperature at a rate of −0.1° C./second. 20 μL of this product was used for single or multiplex fluorescence with 10 μg of Cas9 nuclease (Aldevron, Fargo, N. Dak., USA) and 100 μM of sgRNA in 100 μL at 1× New England BioLabs buffer 3.1 (New England BioLabs, Ipswich, Mass., USA).

Single fluorescence assays were performed in a black 96-well plate (ThermoFisher, Waltham, Mass., USA) and signal was recorded using excitation: 485 nm, emission: 530 on a BioTek (Winooski, Vt.) plate reader. Multiplex fluorescence was performed in a 96-well skirted PCR plate (ThermoFisher, Waltham, Mass., USA) and fluorescence was recorded in the FAM, VIC®, TAMRA (Carboxytetramethylrhodamine), and JUN™ channels every 30 s over one hour using the QuantStudio3 Real-Time PCR System (ThermoFisher, Waltham, Mass., USA).

### RT-PCR

Genomic RNA from severe acute respiratory syndrome-related coronavirus 2 (ATCC® VR-1986D™) was acquired from the American Type Culture Collection (Baltimore, Md.) and was deposited by the Centers for Disease Control and Prevention and obtained through BEI Resources, NIAID, NIH (Biodefense and Emerging Infections Research Resources Repository, National Institute of Allergy and Infectious Diseases, National Institutes of Health) Genomic RNA from SARS-Related Coronavirus 2, Isolate USA-WA1/2020, NR-52285.

Reverse transcription was performed with SuperScript™ IV Reverse Transcriptase Master Mix (ThermoFisher, Waltham, Mass., USA).

Real-time PCR of SARS-Co-V2 cDNA was performed with the 2019-nCoV RUO Kit (IDT, Coralville, Iowa) following the manufacturer's recommendations and using the QuantStudio3 Real-Time PCR System (ThermoFisher, Waltham, Mass., USA). Human glyceraldehyde 3-phosphate dehydrogenase (GAPDH) was detected with the Hs02786624_g1 probe (ThermoFisher, Waltham, Mass., USA).

### Genome Analysis

SARS-CoV-2 genome sequence was obtained from the global initiative on sharing avian influenza data current to January 2021 (www.gisaid.org).

### Graphing and Statistics

Values were graphed using GraphPad Prism 9 (San Diego, Calif., USA) and statistical evaluation was performed using one-way analysis of variance (ANOVA) and a post hoc Tukey's multiple comparisons test.

### Images

Photography was performed with a Canon 5DIII with a Tamron 25-70 lens at the 70 mm setting. Figure images were produced with BioRender.com (Toronto, ON, Canada).

### Results

### Rapid Nucleic Amplification and Lateral Flow Detection

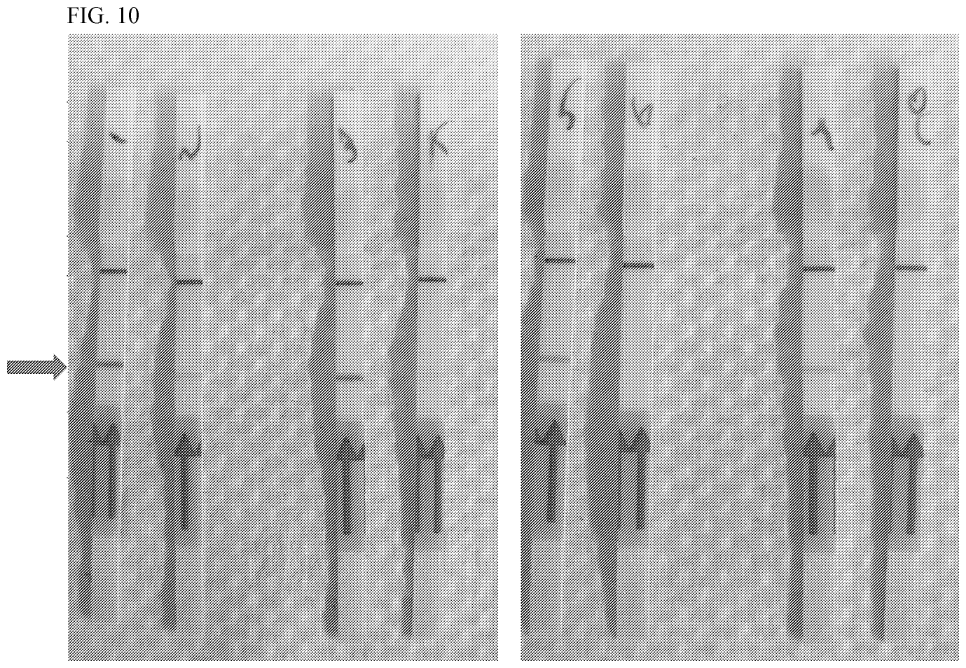

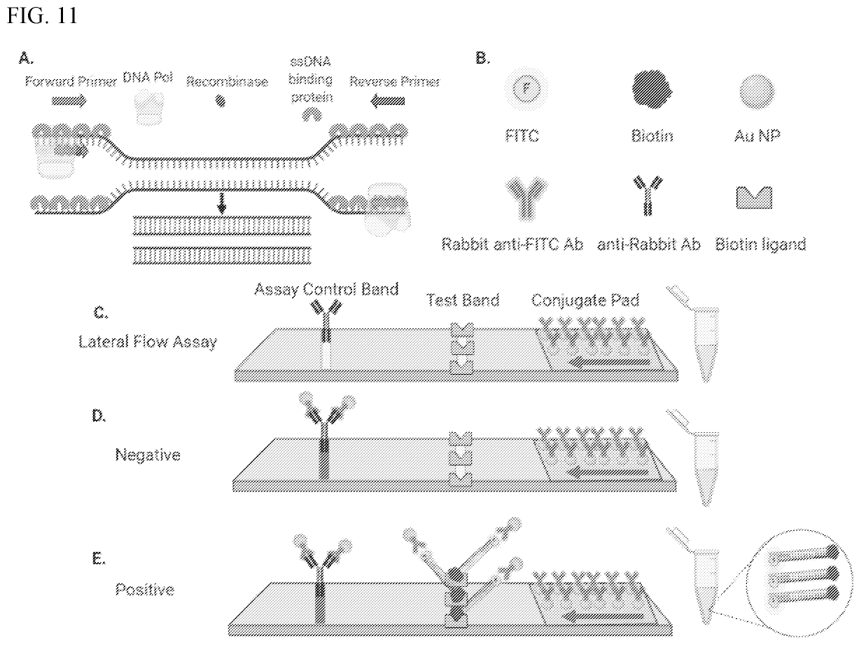

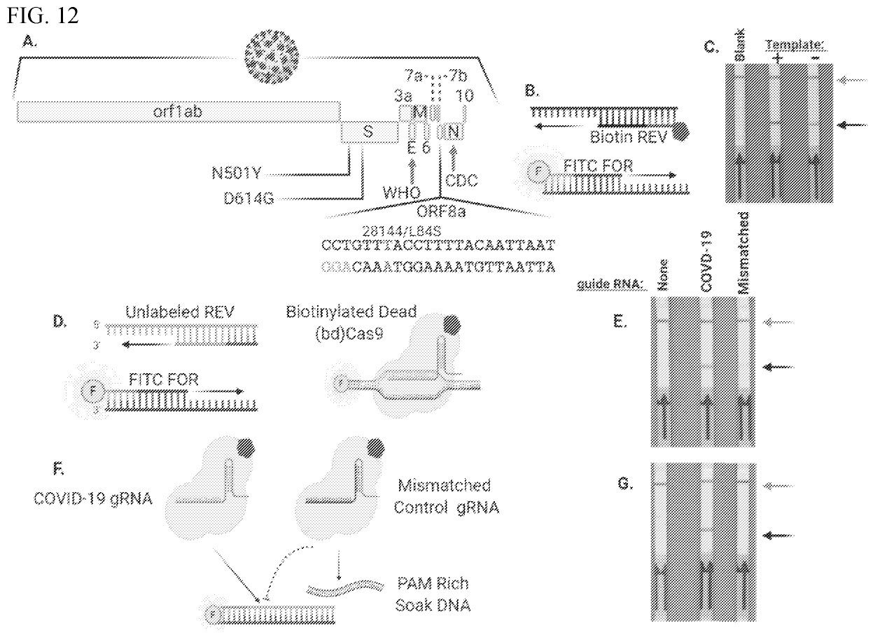

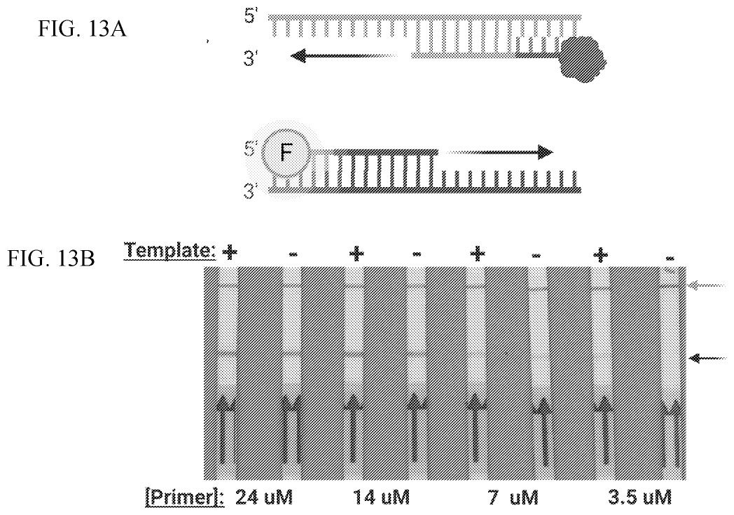

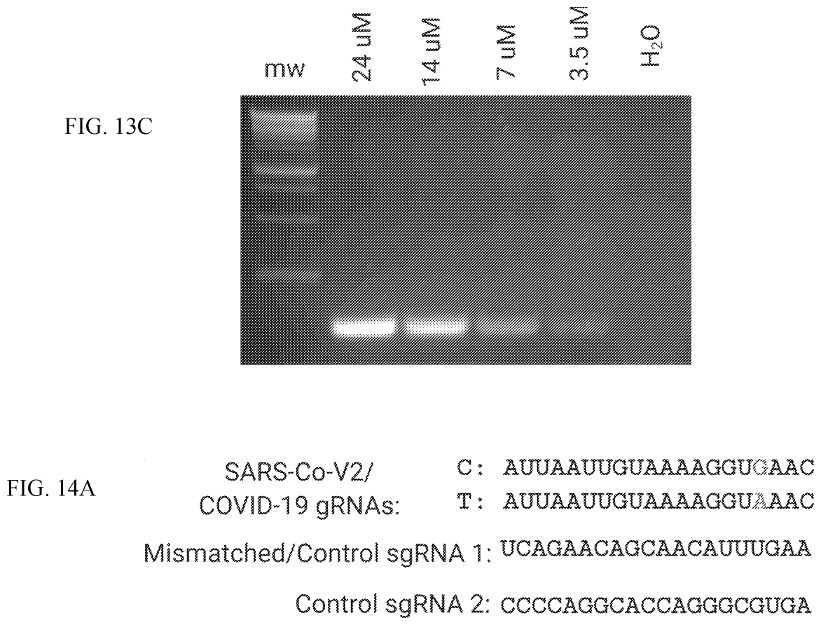

Isothermal RPA amplification and detection via LFA are a simplified approach for nucleic acid analysis that avoids the need for specialized infrastructure (FIG. 11A) [Lobato, I. M.; O'Sullivan, C. K. Recombinase polymerase amplification: Basics, applications and recent advances. Trends Anal. Chem 2018, 98, 19-35, doi:10.1016/j.trac.2017.10.015; and Koczula, K. M.; Gallotta, A. Lateral flow assays. Essays Biochem 2016, 60, 111-120, doi:10.1042/EBC20150012]. The LFA strips employed in this study require test material that is labeled with both FITC/FAM and biotin (FIG. 11B-E). FITC/FAM:biotin conjugated analytes are captured at the test band that contains a biotin ligand (FIG. 11C,E). FITC Au-NPs are in excess and a portion remain unbound and flow to the assay control band (FIG. 11D). This ensures that the LFA test strip is functional and suitable for interpretation. Forward and reverse PCR primers were designed that were labeled with FITC and biotin respectively, to generate SARS-CoV-2 ORF8a gene amplicons that flanked a single nucleotide polymorphism at genomic nucleotide position 28144 that causes a L84S amino acid substitution (FIG. 12A,B). ORF8a is a ˜100 amino acid protein with putative ER import signal sequences [Flower, T. G.; Buffalo, C. Z.; Hooy, R. M.; Allaire, M.; Ren, X.; Hurley, J. H. Structure of SARS-CoV-2 ORFS, a rapidly evolving coronavirus protein implicated in immune evasion. bioRxiv 2020, 10.1101/2020.08.27.270637, doi:10.1101/2020.08.27.270637]. While the Centers for Disease Control and Prevention (CDC) and World Health Organization (WHO) assays detect the N and E genes respectively, this assay was designed for the ORF8a region of the SARS-CoV-2 genome because it does not overlap with other viral sequences and the L84S alteration was the first mutation observed in the USA (FIG. 12A) [Wang, R.; Chen, J.; Gao, K.; Hozumi, Y.; Yin, C.; Wei, G. Characterizing SARS-CoV-2 mutations in the United States. Res. Sq 2020, 10.21203/rs.3.rs-49671/v1, doi:10.21203/rs.3.rs-49671/v1]. This mutation is proximal to a protospacer-adjacent motif (PAM) of −NGG (N=any nucleotide and G=guanine) for the Streptococcus pyogenes Cas9, making it suited for Cas9 detection (FIG. 12A). The use of dual-labeled (i.e., FAM and biotin) primers resulted in positive LFA test bands in either the presence or absence of an ORF8a template (FIG. 12C). It was hypothesized that primer dimers that resulted in a single complex containing FITC and biotin were the cause of this false positive result and it was corrected by titrating the primer concentration. It was observed that higher primer concentrations contributed to dimerization and high false positive rates and lower primer amounts diminished sensitivity (FIGS. 13A-13C). These results showed that direct amplification with labeled primers may be suboptimal for unambiguous nucleic acid detection of ORF8a by LFA.

### Cas9 Allows for Specific SARS-Co-V2 ORF8a Sequence Detection

In order to avoid prohibitively high false positive test results, the labeling of the target amplicon was next approached using a FAM/FITC forward primer and an unlabeled reverse primer (FIG. 12D). It was posited that biotinylation of this FITC-labeled PCR product could be accomplished using a biotinylated, nuclease inactivated (‘dead’) version of Cas9 (bdCas9) (FIG. 12D). FITC ORF8a amplicons generated by standard PCR were incubated with bdCas9 and an ORF8a-specific sgRNA (FIG. 12A) or a mismatched control sgRNA (FIG. 14A). Under these conditions, a readily observable test band was seen using the COVID-19 sgRNA and a faint one using the control sgRNA with no homology to SARS-Co-V2 (FIG. 12E). Densitometry has been applied to ascribe values for semi-quantitative results as well as to differentiate the test bands of experimental and controls [Huang, X.; Aguilar, Z. P.; Xu, H.; Lai, W.; Xiong, Y. Membrane-based lateral flow immunochromatographic strip with nanoparticles as reporters for detection: A review. Biosens Bioelectron 2016, 75, 166-180, doi:10.1016/j.bios.2015.08.032]. Because Cas9 is physically associated with DNA while scanning for target sites to cleave [Mekler, V.; Minakhin, L.; Severinov, K. Mechanism of duplex DNA destabilization by RNA-guided Cas9 nuclease during target interrogation. Proc. Natl Acad Sci USA 2017, 114, 5443-5448, doi:10.1073/pnas.1619926114], it was predicted that this interaction would led to non-specific DNA labeling and a faint but visible test band in the control sgRNA (FIG. 12E). Therefore, to improve definitive interpretation, a competing ‘soak’ DNA that was rich in PAM-NGG sequences was designed in order to prevent indiscriminate Cas9:DNA interactions (FIG. 12F and FIG. 14B). This allowed for specific detection using a COVID-19 sgRNA, while the control mismatched sgRNA did not show a test band, therefore false positive detection was avoided (FIG. 12G). Conversely, an irrelevant amplicon was not recognized by the COVID-19 sgRNA (FIG. 14C). These results confirmed the ability of bdCas9 to label a FITC amplicon for detection via LFA.

To merge the capabilities of RPA and our bdCas9-based detection method, the conditions for rapid SARS-Co-V2 sequence detection was investigated. First, RPA using a FITC-labeled forward and unlabeled reverse primer was performed in the presence of bdCas9. Even with a large excess of competitor soak DNA, a positive test band was observed on LFA strips with either COVID or control sgRNAs when a SARS-Co-V2 DNA template was present (FIG. 15A). The two reaction components were then separated, first by performing a 20 min RPA at room temperature and then using RPA products for bdCas9 detection. With a 20 min bdCas9 incubation, test bands were visible that became more defined at 40 and 60 min and with increasing amounts of soak DNA (FIG. 15B). Collectively, these data showed that SARS-CoV-2 DNA amplified by RPA can be detected with bdCas9 and LFA in the presence of a competitor/soak DNA.

### Cas9-Nuclease-Based Diagnostics for Single and Multiplexed Targets

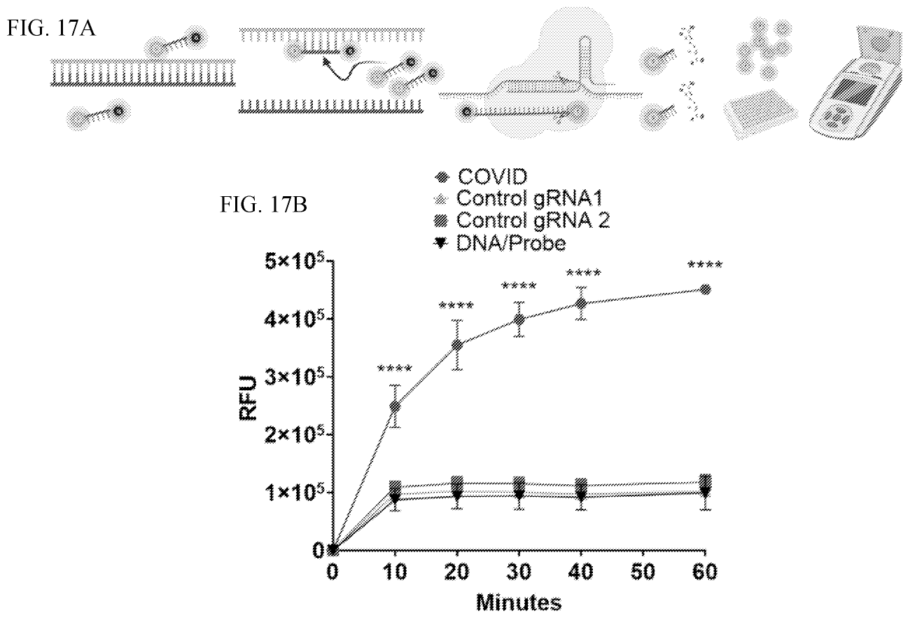

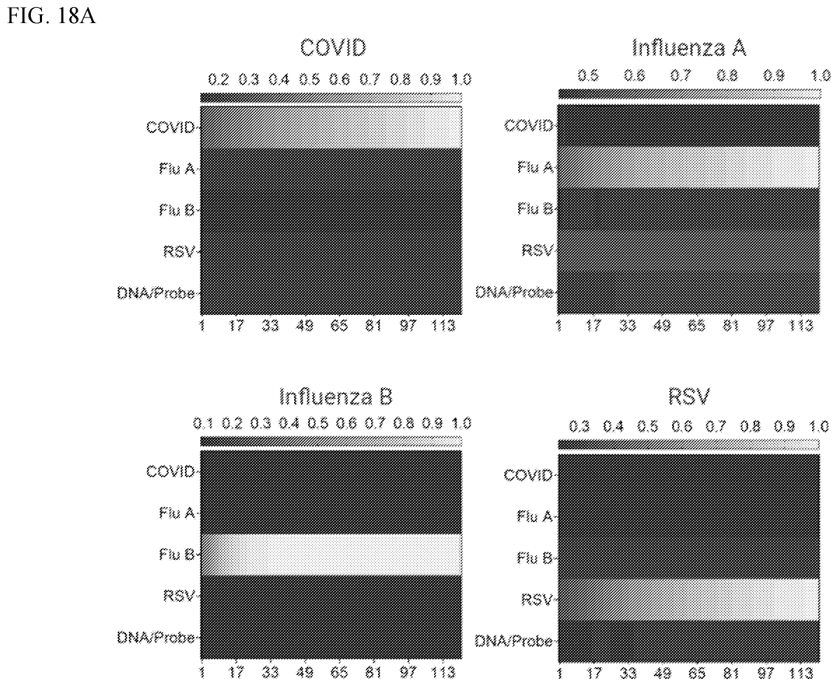

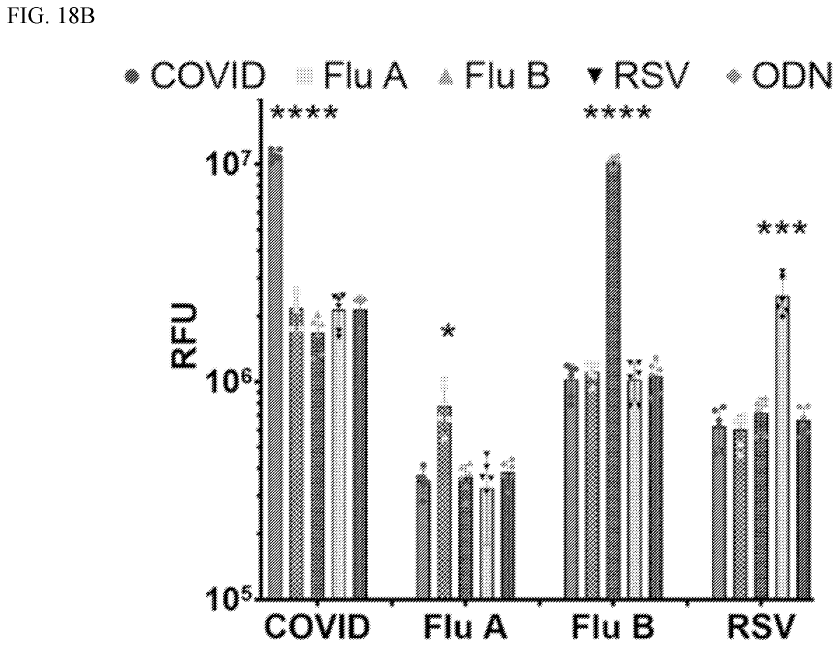

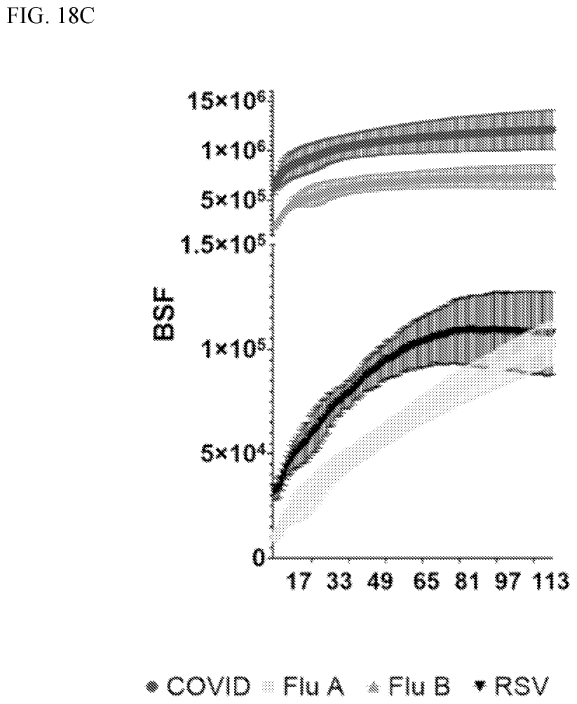

The nuclease properties of Cas9 also hold potential to serve as a diagnostic platform by cleaving a fluorescent probe in a sequence-specific manner (FIG. 17A). COVID-19-specific probes labeled with a fluorescent marker and quencher were designed, that when hybridized with SARS-CoV-2 amplicons, were cleaved by Cas9 nuclease together with a COVID-19 sgRNA (FIG. 17B). The ability to multiplex Cas9 with multiple sgRNAs also allowed us to test whether we could achieve simultaneous detection of viruses with overlapping symptomology. First, we designed, built, and tested DNA probes with distinct fluorophores for SARS-CoV-2, influenza A and B, and RSV, respectively. These were tested and showed specificity of fluorescent signaling only for matched sgRNAs (FIG. 18A, 18B). Next, the four distinct viral detection components were all combined in a single reaction mixture and analyzed simultaneously under isothermal (37° C.) conditions in a single tube using real-time fluorometry with a standard quantitative PCR instrument in a 96-well format (FIG. 18C). Distinct fluorescence signals from cleavage of the pathogen-specific probe by the disease-specific sgRNA were observed. These results showed that all four viral pathogens could be detected in a multiplex fashion.

LFA and Fluorescence Assay Validation with SARS-CoV-2 Genomic RNA

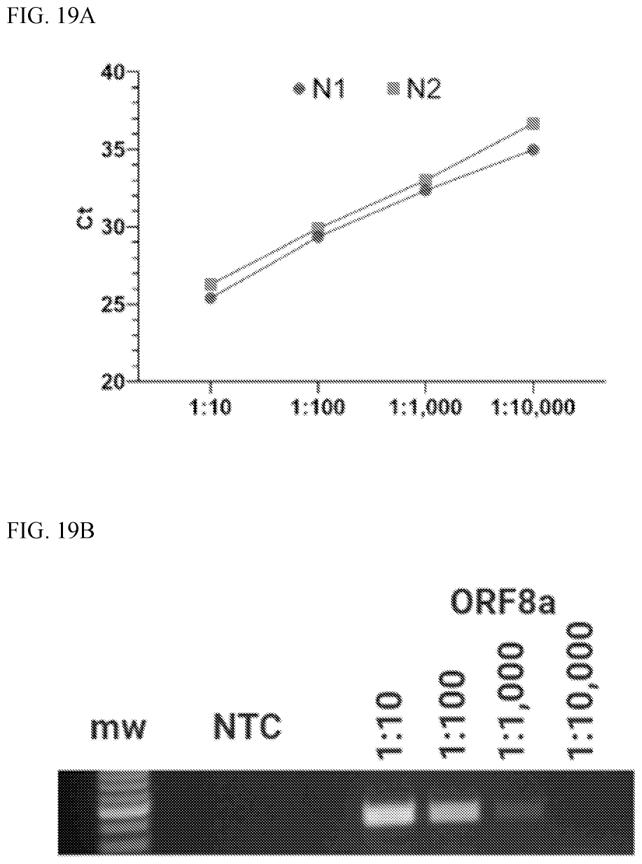

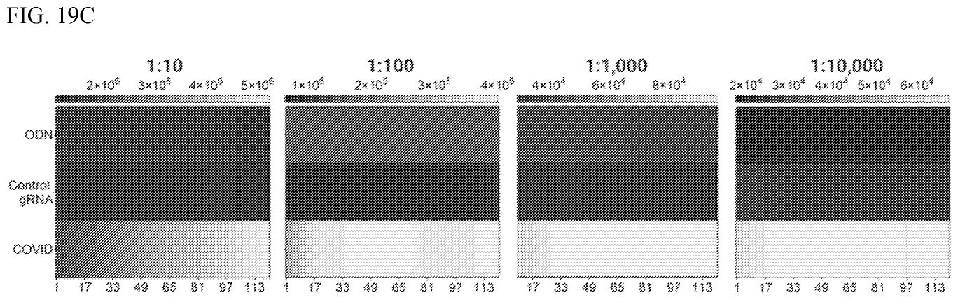

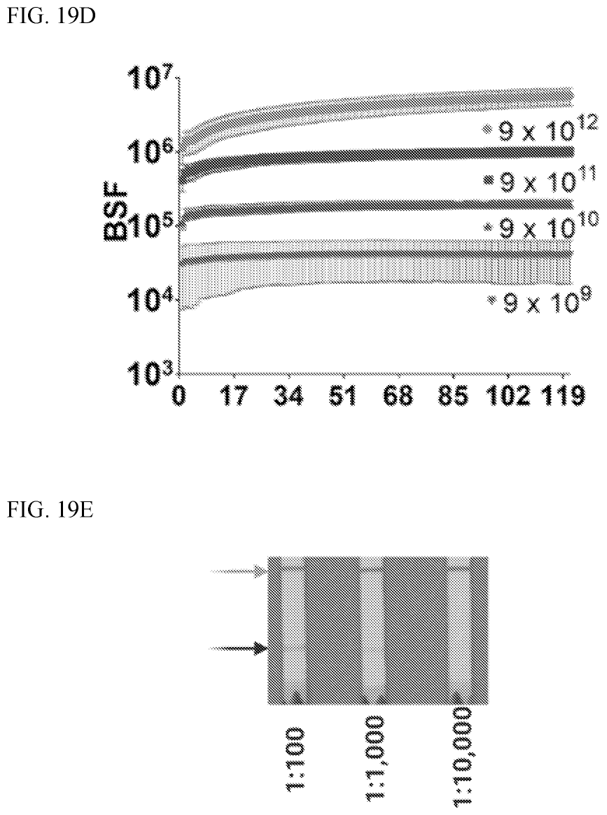

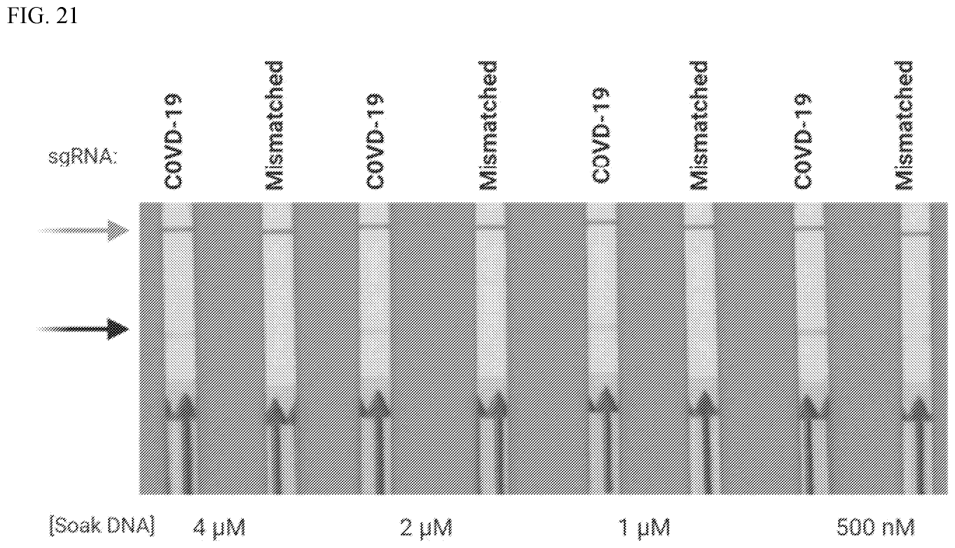

Genomic RNA from the USA-WA1/2020 isolate was diluted and reverse transcribed followed by qRT PCR with the designated primer:probes employed by the Centers for Disease Control and Prevention for the N gene (FIGS. 12A and 19A, and FIG. 20). Using ORF8a primers, the same dilution series was performed and resolved by gel electrophoresis (FIG. 19B). This amplicon was then used for establishing the limit of detection (LOD) with the fluorescence and LFAs. Fluorescence signal above background was observed for each sample, indicating that the Cas9-based assay limit of detection (LOD) was similar to that of qRT-PCR (FIGS. 19A, 19C). We further quantified ORF8a amplicons and performed a copy number LOD. Fluorescence above background was observed with 9×10⁹ copies of target DNA (FIG. 19D). Under the parameters of 35 cycle exponential PCR amplification (2³⁵), this represents <5 copies of starting material. LFA optimization with soak DNA and irrelevant sgRNA was performed (FIG. 21). Under these conditions, test bands were observable at a sensitivity that was an order of magnitude below that of qRT-PCR or Cas9 fluorescence (FIG. 19E). Together, these results show that LFA and Cas9 fluorescence can be used for SARS-CoV-2 viral nucleic acid detection.

### Cas9 Analysis of a SARS-CoV-2 Variant

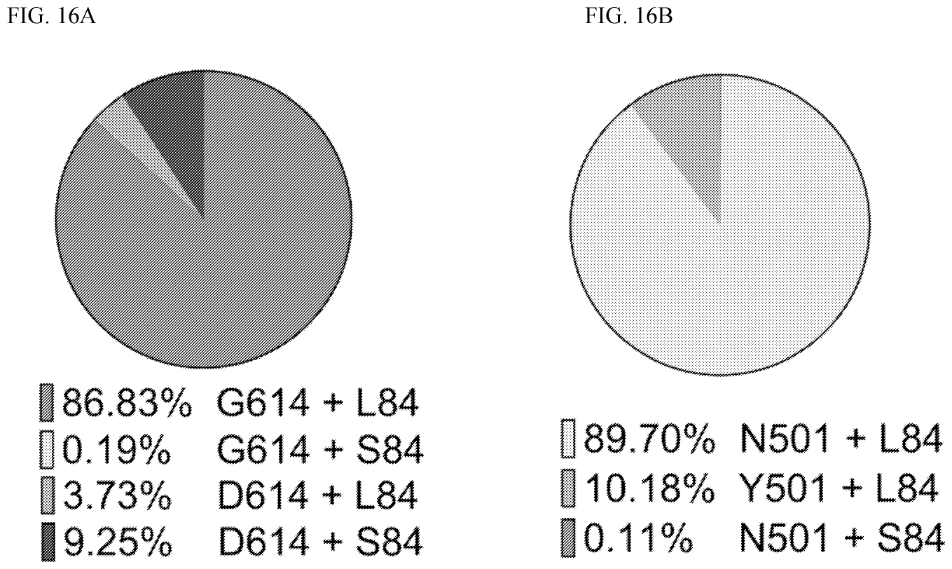

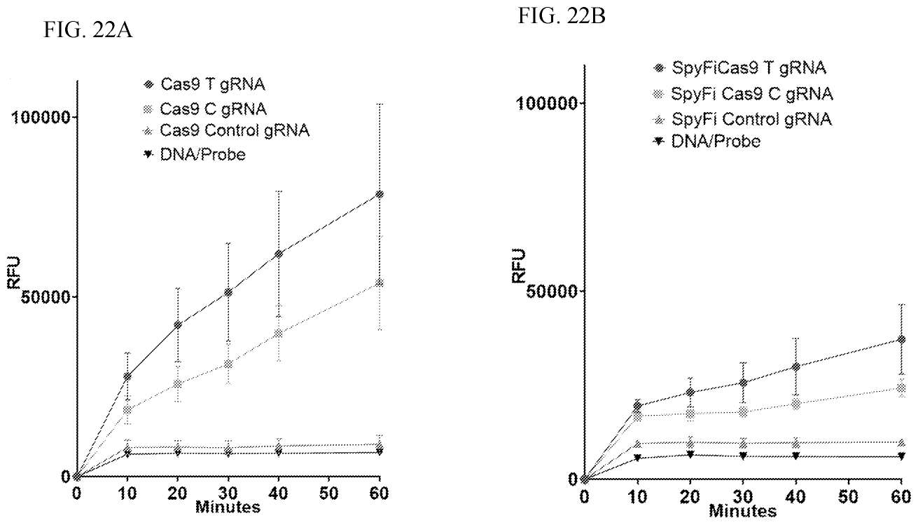

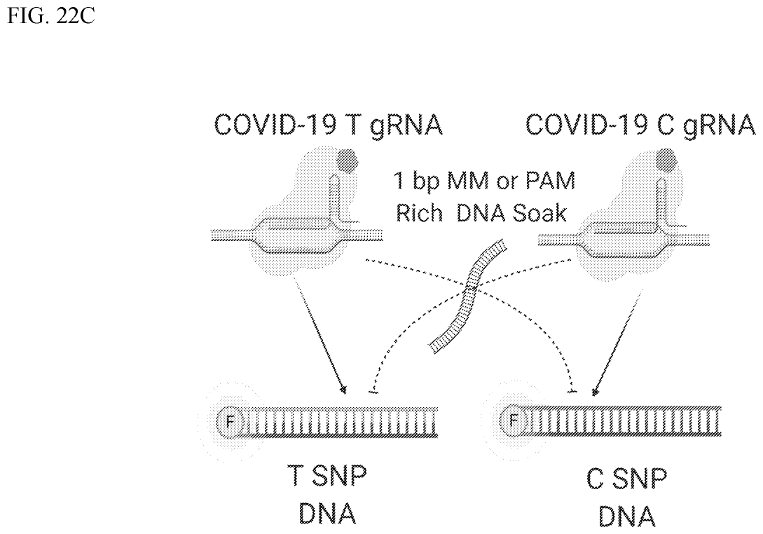

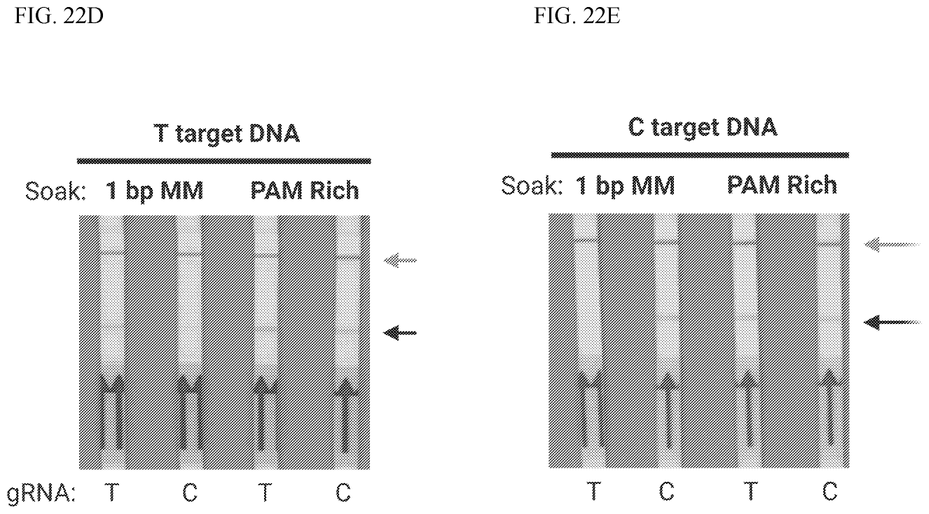

Amino acid substitutions D614G and N501Y in the S gene and L84S in ORF8a have been suggested to result in increased viral load [Plante, J. A.; Liu, Y.; Liu, J.; Xia, H.; Johnson, B. A.; Lokugamage, K. G.; Zhang, X.; Muruato, A. E.; Zou, J.; Fontes-Garfias, C. R., et al. Spike mutation D614G alters SARS-CoV-2 fitness. Nature 2020, 10.1038/s41586-020-2895-3, doi:10.1038/s41586-020-2895-3; and Liangsheng Zhang, J.-R. Y., Zhenguo Zhang, Zhenguo Lin. Genomic variations of SARS-CoV-2 suggest multiple outbreak sources of transmission. MedRxiv 2020, https://doi.org/10.1101/2020.02.25.20027953, doi:https://doi.org/10.1101/2020.02.25.20027953] S gene D614G is highly prevalent [Hou, Y. J.; Chiba, S.; Halfmann, P.; Ehre, C.; Kuroda, M.; Dinnon, K. H., 3rd; Leist, S. R.; Schafer, A.; Nakajima, N.; Takahashi, K., et al. SARS-CoV-2 D614G variant exhibits efficient replication ex vivo and transmission in vivo. Science 2020, 10.1126/science.abe8499, doi:10.1126/science.abe8499] and N501Y has led to COVID surges [Tang, J. W.; Toovey, O. T. R.; Harvey, K. N.; Hui, D. D. S. Introduction of the South African SARS-CoV-2 variant 501Y.V2 into the UK. J. Infect. 2021, 10.1016/j.jinf2021.01.007, doi:10.1016/j.jinf2021.01.007]. Being able to distinguish SARS-CoV-2 strains may aid in whether certain strains are associated with differential clinical outcomes and/or could provide rapid information to public health departments. However, because the S gene between coronaviruses are highly homologous, targeting it was avoided with CRISPR/Cas9 to avoid false positives that may occur from a coronavirus other than SARS Co-V2. Instead, L84S was targeted, caused by a SNP in the ORF8a gene that is unique to SARS-Co-V2 and delineated the relationships between S gene D614G, N501Y, and ORF8a L84S (FIG. 12A and FIGS. 16A and 16B). The ability of Cas9 nuclease and bdCas9 to distinguish between cytosine and thymine at nucleotide position 28144 in ORF8a was determined (FIG. 2A). The sequence of an ORF8a DNA amplicon was interrogated with a perfectly matched fluorescent probe and sgRNAs with matched complementarity (thymine) or a one base pair mismatch (cytosine). Either wild-type or a high-fidelity version of Cas9 (SpyFi™) [Vakulskas, C. A.; Dever, D. P.; Rettig, G. R.; Turk, R.; Jacobi, A. M.; Collingwood, M. A.; Bode, N. M.; McNeill, M. S.; Yan, S.; Camarena, J., et al. A high-fidelity Cas9 mutant delivered as a ribonucleoprotein complex enables efficient gene editing in human hematopoietic stem and progenitor cells. Nat. Med. 2018, 24, 1216-1224, doi:10.1038/s41591-018-0137-0] were employed, and each showed differential fluorescence signal between matched and mismatched; however, it was not statistically significant (FIG. 22A, 22B). The ability of bdCas9 in the LFA assay to distinguish targets at the single nucleotide level was then determined. For this, two soak DNA candidates were utilized: the PAM-rich soak DNA or a more homologous competitor soak that differed from the target and sgRNA by a single nucleotide (FIG. 14B and FIG. 22C). A test band was observed for both the perfectly matched and one base pair mismatched DNA target when the PAM-rich soak was used (FIGS. 22D and 22E). In contrast, when using soak DNA containing the single nucleotide mismatch from the target, only the sgRNA with perfect match to the target yielded a test band (FIGS. 22D and 22E). These data showed that, using an appropriate soak DNA sequence, bdCas9 and LFA could resolve DNA targets at the single nucleotide level.

### 4. Discussion

The granting of emergency use authorization of SARS-Co-V2 vaccines represents a hopeful end to a pandemic that has infected more than 100 million people worldwide and claimed greater than 2 million lives from January 2020 to January 2021. It is predicted that widespread vaccine administration will not be available until the second or third quarter of 2021, making continued testing and mitigation efforts crucial to minimize more loss of life and continued global social, economic, and in-person schooling disruptions.
We set out to leverage the ability and specificity of Streptococcus pyogenes Cas9 to interrogate and identify SARS-Co-V2 sequences to develop testing platforms for both field-based and more specialized laboratory testing. The former requires simplified methodologies and rapid readouts and would be particularly helpful in rural areas that lack laboratory facilities able to perform molecular diagnostics. Rural COVID-19 case rates are increasing [Paul, R.; Arif, A. A.; Adeyemi, O.; Ghosh, S.; Han, D. Progression of COVID-19 From Urban to Rural Areas in the United States: A Spatiotemporal Analysis of Prevalence Rates. J. Rural Health 2020, 36, 591-601, doi:10.1111/jrh.12486], rural residents are at elevated risk of COVID-19-related serious illness [Kaufman, B. G.; Whitaker, R.; Pink, G.; Holmes, G. M. Half of Rural Residents at High Risk of Serious Illness Due to COVID-19, Creating Stress on Rural Hospitals. J. Rural Health 2020, 36, 584-590, doi:10.1111/jrh.12481], medical care capacity in lowly populated areas can be quickly overwhelmed [Davoodi, N. M.; Healy, M.; Goldberg, E. M. Rural America's Hospitals are Not Prepared to Protect Older Adults From a Surge in COVID-19 Cases. Gerontol Geriatr Med. 2020, 6, 2333721420936168, doi:10.1177/2333721420936168], and testing is challenging due to a lack of local facilities and funding [Souch, J. M.; Cossman, J. S. A Commentary on Rural-Urban Disparities in COVID-19 Testing Rates per 100,000 and Risk Factors. J. Rural Health 2020, 10.1111/jrh.12450, doi:10.1111/jrh.12450]. To address these testing shortfalls, we employed Cas9 for targeting a portion of the SARS-CoV-2 ORF8a gene (FIG. 2A). Presently, the CDC and WHO test for sequences in the N and E genes, respectively (FIG. 2A). We chose ORF8a for our targeting strategies because there are seven coronaviruses that infect humans [Nickbakhsh, S.; Ho, A.; Marques, D. F. P.; McMenamin, J.; Gunson, R. N.; Murcia, P. R. Epidemiology of Seasonal Coronaviruses: Establishing the Context for the Emergence of Coronavirus Disease 2019. J. Infect. Dis 2020, 222, 17-25, doi:10.1093/infdis/jiaa185] and the ORF8a sequence is dissimilar between them, making it an ideal gene to target for SARS-CoV-2 detection without false positives occurring from these other viruses. Further, this site was ideal for designing a CRISPR/Cas9 sgRNA with an −NGG PAM (FIG. 12A). The LFA test strips employed in our study require dual labeling of a candidate molecule for detection that is based on capture of the analyte by embedded Au-NPs coated with rabbit anti-FITC antibodies (FIG. 11B, 11C). If the test material lacks a biotin label, it flows past the test band that contains a biotin ligand and accumulates at the assay control band that is coated with anti-rabbit antibodies. The presence of this band, that we termed the assay control, ensures that the LFA reagents and test stick device are functioning properly for detection (FIG. 11D). If the reaction components are labeled with both FITC and biotin, a positive result is observed following Au-NP accumulation at the test band (FIG. 11E). Using recombinase polymerase amplification (RPA) that allows for isothermal amplification of nucleic acids, we observed that the use of PCR primers labeled with FITC and biotin resulted in false positive test bands as a result of primer dimerization (FIG. 12B, 1C and FIG. 13). In order to prevent this, we employed an amplification strategy using one FITC/FAM-labeled and one unlabeled primer (FIG. 12D). To achieve the requisite FITC and biotin conjugation for LFA detection, we employed a nuclease inactive, biotinylated Cas9 with SARS-CoV-2 (COVID-19) or control sgRNAs. Under these conditions, we avoided the false positives resulting from primer dimers; however, control sgRNA with no homology to ORF8a showed the presence of a test band (FIG. 12E). Cas9 can remain stably bound to DNA as it scans for sequences of homology required to initiate DNA cleavage [Ivanov, I. E.; Wright, A. V.; Cofsky, J. C.; Aris, K. D. P.; Doudna, J. A.; Bryant, Z. Cas9 interrogates DNA in discrete steps modulated by mismatches and supercoiling. Proc. Natl Acad Sci USA 2020, 117, 5853-5860, doi:10.1073/pnas.1913445117]. In the absence of nucleolytic properties, such as with ‘dead’ Cas9, DNA is also scanned and the on/off rate is rapid and impacted by Cas9 concentration [Jones, D. L.; Leroy, P.; Unoson, C.; Fange, D.; Curic, V.; Lawson, M. J.; Elf, J. Kinetics of dCas9 target search in Escherichia coli. Science 2017, 357, 1420-1424, doi:10.1126/science.aah7084]. Therefore, the false positive test bands we observed may be due to high concentration of Cas9 that can associate and dissociate with the former, leading to test bands irrespective of the sgRNA. To remedy this, a bait or “soak” DNA sequence comprised of PAM-rich sequences was designed for inclusion in the assay in order to sequester non-specific binding events (FIG. 12 and FIG. 14). Under these new conditions, LFA test bands were observed using the COVID-19 but not the mismatched control sgRNA (FIG. 12G).

To further define the conditions for field-based testing, we explored the optimal settings for rapid amplification and detection via LFA. Previous studies using Cas enzymes have employed LAMP PCR and Cas in a ‘one pot’ approach for simultaneous amplification and detection [Joung, J.; Ladha, A.; Saito, M.; Kim, N. G.; Woolley, A. E.; Segel, M.; Barretto, R. P. J.; Ranu, A.; Macrae, R. K.; Faure, G., et al. Detection of SARS-CoV-2 with SHERLOCK One-Pot Testing. N Engl J. Med. 2020, 383, 1492-1494, doi:10.1056/NEJMc202617; and Woo, C. H.; Jang, S.; Shin, G.; Jung, G. Y.; Lee, J. W. Sensitive fluorescence detection of SARS-CoV-2 RNA in clinical samples via one-pot isothermal ligation and transcription. Nat. Biomed. Eng. 2020, 4, 1168-1179, doi:10.1038/s41551-020-00617-5]. In our system, this strategy yielded high levels of false positives even in the presence of large amounts of competitor soak DNA (FIG. 15A). In order to prevent this, isothermal RPA was performed followed by biotinylated Cas9 interrogation. Increased amounts of soak DNA and longer dbCas9:sgRNA:DNA incubation times from 20 to 60 min improved the resolution of detection (FIG. 15B). Centralized reference and public health diagnostic sites can still face backlogs, leading to increased turnaround time for results. Therefore, we sought to apply CRISPR/Cas9 toward developing a higher throughput methodology. The design was based on the nuclease properties of Cas9 to cleave a fluorescent probe hybridized to a target amplicon (FIG. 17A). Following a 20 min isothermal room temperature RPA, a fluorescently labeled SARS-CoV-2 probe was annealed to the reaction product and incubated with Cas9 peptide and an ORF8a sgRNA. This resulted in rapid fluorescence generation with a statistically significant difference between Cas9 with COVID-19 sgRNA and control and DNA:probe reactions observable in as little as 10 minutes (FIG. 17B).

COVID-19 symptoms can mirror those of influenza, which is most prevalent during the winter months in the northern hemisphere, and it is possible for one infection to be confused for another or for co-infection with both agents to occur [Wu, X.; Cai, Y.; Huang, X.; Yu, X.; Zhao, L.; Wang, F.; Li, Q.; Gu, S.; Xu, T.; Li, Y., et al. Co-infection with SARS-CoV-2 and Influenza A Virus in Patient with Pneumonia, China. Emerg Infect. Dis 2020, 26, 1324-1326, doi:10.3201/eid2606.200299]. Therefore, the CDC has designed multiplex qRT-PCR assays capable of detecting multiple viral pathogens in the same sample [Waggoner, J. J.; Stittleburg, V.; Pond, R.; Saklawi, Y.; Sahoo, M. K.; Babiker, A.; Hussaini, L.; Kraft, C. S.; Pinsky, B. A.; Anderson, E. J., et al. Triplex Real-Time RT-PCR for Severe Acute Respiratory Syndrome Coronavirus 2. Emerg Infect. Dis 2020, 26, 1633-1635, doi:10.3201/eid2607.201285]. We likewise assessed the multiplex capability of Cas9 to detect and distinguish SARS-CoV-2, influenza A and B, and further designed and added components for detecting RSV. Individual sgRNAs were first tested against specific pathogen DNA:probe hybrids to assess whether there was any cross-reactivity. FIG. 18A shows that only the Cas9:sgRNA complex matched to the target DNA:probe generated a distinct fluorescent signal. These data and those of FIG. 2C,E are complementary and support the mechanism of Cas that interacts with and scans DNA in a broad manner but cleaves DNA in a sequence-specific fashion [Knight, S. C.; Xie, L.; Deng, W.; Guglielmi, B.; Witkowsky, L. B.; Bosanac, L.; Zhang, E. T.; El Beheiry, M.; Masson, J. B.; Dahan, M., et al. Dynamics of CRISPR-Cas9 genome interrogation in living cells. Science 2015, 350, 823-826, doi:10.1126/science.aac6572]. As such, in the bdCas9 binding assay, a competitor soak DNA is required to inhibit this process in order to achieve specificity by LFA, while the nuclease-dependent fluorescent approach is capable of specificity without competitor soak DNA (FIGS. 12 and 18A). The detection kinetics for each target were distinguishable from mismatched sgRNA sample fluorescence signal (FIG. 18B). Because no promiscuous activity was observed between any sgRNA, the amplicon:probe hybrids were then pooled for single-well multiplex analysis. Rapid fluorescence was generated that was readily distinguishable from controls (FIG. 18C), showing the potential of CRISPR/Cas9 nuclease-based diagnostics. Importantly, this method can be performed on a quantitative PCR instrument, and while this represents specialized equipment, it is also standard equipment in many research laboratories and most public health facilities. The speed of detection, capability to multiplex, and the ability to perform reactions in a 96-well format (or greater) makes this a scalable platform for high-throughput analytics.

With proof-of-principle established for our methodology using synthetic fragments, we then validated both LFA and fluorescence detection using the USA-WA1/2020 coronaviral isolate. A dilution series was performed using the CDC qRT PCR assay with the N1 and N2 primer:probe set and the 1:10,000 dilution showed a Ct of 34.9±0.78 for N1 and 36.7±0.55 for N2 (FIG. 19A and FIG. 20). Our CRISPR/Cas9 fluorescence assay was also able to detect the 1:10,000 dilution above background (FIG. 19C). CDC guidelines are for positive tests to have a Ct<40 [Lu, X.; Wang, L.; Sakthivel, S. K.; Whitaker, B.; Murray, J.; Kamili, S.; Lynch, B.; Malapati, L.; Burke, S. A.; Harcourt, J., et al. US CDC Real-Time Reverse Transcription PCR Panel for Detection of Severe Acute Respiratory Syndrome Coronavirus 2. Emerg Infect. Dis 2020, 26, doi:10.3201/eid2608.201246] and other studies show reduced ability to isolate virus when Ct values exceed 35 [Singanayagam, A.; Patel, M.; Charlett, A.; Lopez Bernal, J.; Saliba, V.; Ellis, J.; Ladhani, S.; Zambon, M.; Gopal, R. Duration of infectiousness and correlation with RT-PCR cycle threshold values in cases of COVID-19, England, January to May 2020. Euro Surveill 2020, 25, doi:10.2807/1560-7917.ES.2020.25.32.2001483]. Thus, our fluorescence assay is comparable to qRT-PCR with a LOD that correlates to a Ct of ˜35. In contrast, the LOD of LFA was an order of magnitude lower (FIG. 19E), which is in keeping with its detection by visualization vs fluorescence, making it less sensitive, and may result in an inability to detect patients with low viral titers and makes follow-on confirmation of rapid tests important. To facilitate streamlined confirmation of LFAs, part of our design strategy was to employ a reverse transcription step using oligo dT/random hexamer priming. This allows for the same sample to be tested by LFA, fluorescence, and/or qRT-PCR. This is differential to some ‘one-pot’ approaches, such as RT LAMP; however, using gene-specific priming in this manner, we observed unacceptably high false priming events during PCR (data not shown). Moreover, the use of oligo dT/hexamer priming allows for standard control gene analysis during confirmation testing using nucleic acid amplification tests in accordance with CDC and WHO guidelines. Additionally, our RT strategy will support whole-genome sequencing that will provide further knowledge on coronaviral strain distribution and prevalence [McNamara, R. P.; Caro-Vegas, C.; Landis, J. T.; Moorad, R.; Pluta, L. J.; Eason, A. B.; Thompson, C.; Bailey, A.; Villamor, F. C. S.; Lange, P. T., et al. High-Density Amplicon Sequencing Identifies Community Spread and Ongoing Evolution of SARS-CoV-2 in the Southern United States. Cell Rep. 2020, 33, 108352, doi:10.1016/j.celrep.2020.108352]. Follow-on confirmation testing is critical for any field-based assay, particularly for our LFA that does not generate/evaluate a human control gene. Rather, the observed assay control band shows proper function of the LFA strips. Future improvements to our approach will include a human control gene that is differentially labeled such that viral target and control genes can be analyzed on the same LFA.

Coronaviruses have genetic proofreading systems [Sevajol, M.; Subissi, L.; Decroly, E.; Canard, B.; Imbert, I. Insights into RNA synthesis, capping, and proofreading mechanisms of SARS-coronavirus. Virus Res. 2014, 194, 90-99, doi:10.1016/j.virusres.2014.10.008]; however, mutations occur with potential to confer favorable properties, including increased infectivity or to diminish diagnostic capabilities [Pereira, F. SARS-CoV-2 variants lacking a functional ORF8 may reduce accuracy of serological testing. J. Immunol Methods 2021, 488, 112906, doi:10.1016/j.jim.2020.112906]. S gene mutations such as D614G or N501Y and ORF8a L84S are suggested to have higher rates of infectivity [Plante, J. A.; Liu, Y.; Liu, J.; Xia, H.; Johnson, B. A.; Lokugamage, K. G.; Zhang, X.; Muruato, A. E.; Zou, J.; Fontes-Garfias, C. R., et al. Spike mutation D614G alters SARS-CoV-2 fitness. Nature 2020, 10.1038/s41586-020-2895-3, doi:10.1038/s41586-020-2895-3; Liangsheng Zhang, J.-R. Y., Zhenguo Zhang, Zhenguo Lin. Genomic variations of SARS-CoV-2 suggest multiple outbreak sources of transmission. MedRxiv 2020, https://doi.org/10.1101/2020.02.25.20027953, doi:https://doi.org/10.1101/2020.02.25.20027953; and Hou, Y. J.; Chiba, S.; Halfmann, P.; Ehre, C.; Kuroda, M.; Dinnon, K. H., 3rd; Leist, S. R.; Schafer, A.; Nakajima, N.; Takahashi, K., et al. SARS-CoV-2 D614G variant exhibits efficient replication ex vivo and transmission in vivo. Science 2020, 10.1126/science.abe8499, doi:10.1126/science.abe8499]. The S genes in which the D614 and N501Y residues reside are homologous between SARS-CoV and SARS-CoV-2 [Gralinski, L. E.; Menachery, V. D. Return of the Coronavirus: 2019-nCoV. Viruses 2020, 12, doi:10.3390/v12020135]. In contrast, the ORF8a gene shows little homology between coronaviruses and the L846 polymorphism is in the seed sequence of the sgRNA using the CRISPR/Cas9 enzyme from Streptococcus pyogenes (FIG. 12A). The seed sequence is the first ˜10 bp proximal to the PAM and dictates specificity to a higher degree than PAM distal sequences [Wu, X.; Scott, D. A.; Kriz, A. J.; Chiu, A. C.; Hsu, P. D.; Dadon, D. B.; Cheng, A. W.; Trevino, A. E.; Konermann, S.; Chen, S., et al. Genome-wide binding of the CRISPR endonuclease Cas9 in mammalian cells. Nat. Biotechnol 2014, 32, 670-676, doi:10.1038/nbt.2889; Jinek, M.; Chylinski, K.; Fonfara, I.; Hauer, M.; Doudna, J. A.; Charpentier, E. A programmable dual-RNA-guided DNA endonuclease in adaptive bacterial immunity. Science 2012, 337, 816-821, doi:10.1126/science.1225829; Sternberg, S. H.; Redding, S.; Jinek, M.; Greene, E. C.; Doudna, J. A. DNA interrogation by the CRISPR RNA-guided endonuclease Cas9. Nature 2014, 507, 62-67, doi:10.1038/nature13011; Cong, L.; Ran, F. A.; Cox, D.; Lin, S.; Barretto, R.; Habib, N.; Hsu, P. D.; Wu, X.; Jiang, W.; Marraffini, L. A., et al. Multiplex genome engineering using CRISPR/Cas systems. Science 2013, 339, 819-823, doi:10.1126/science.1231143; and Jiang, W.; Bikard, D.; Cox, D.; Zhang, F.; Marraffini, L. A. RNA-guided editing of bacterial genomes using CRISPR-Cas systems. Nat. Biotechnol 2013, 31, 233-239, doi:10.1038/nbt.2508]. These properties make ORF8a a desirable target site for overall specificity and single nucleotide analysis. Using wild-type or a high-fidelity Cas9, we assessed the single nucleotide distinction capabilities of Cas9 nuclease for ORF8a L84 (thymine) or S84 (cytosine) in our fluorometric assay. These data showed a trend for each enzyme in generating higher fluorescence signals when perfect homology between the DNA:sgRNA was present; however, the differences were not statistically significant (FIGS. 22A, 22B). In contrast, analysis with bdCas9 and LFA allowed for single-nucleotide resolution when a soak DNA was included that was mismatched from the target DNA sgRNA site by 1 bp (FIGS. 22C-22E and FIG. 14B). Being able to rapidly obtain information on viral polymorphisms could greatly enhance the ability to track the spread or infectivity of novel viral strains. The mutational rate of SARS-CoV-2 shows hotspots in the Orf1ab [Laamarti, M.; Alouane, T.; Kartti, S.; Chemao-Elfihri, M. W.; Hakmi, M.; Essabbar, A.; Laamarti, M.; Hlali, H.; Bendani, H.; Boumajdi, N., et al. Large scale genomic analysis of 3067 SARS-CoV-2 genomes reveals a clonal geo-distribution and a rich genetic variations of hotspots mutations. Plos One 2020, 15, e0240345, doi:10.1371/journal.pone.0240345] gene, which may owe to its larger size, and diagnostics that require sequence specificity such as ours may be invalidated should a target site be mutated [Pereira, F. SARS-CoV-2 variants lacking a functional ORF8 may reduce accuracy of serological testing. J. Immunol Methods 2021, 488, 112906, doi:10.1016/j.jim.2020.112906]. However, the plasticity of CRISPR/Cas9 targeting allows for the rapid development and deployment of new reagents to circumvent this. Further, our deliberate use of wholly commercial reagents, most of which are obtained lyophilized and therefore highly stable, supports the development and archival of assays and reagents for current and emergent biological threat events.

In this study, we showed proof-of-principle for Cas9 in detecting target sequences for analysis by LFA or fluorometry. Single nucleotide resolution by LFA can aid in strain identification and has broad applicability for rapidly assessing circulating viral pathogens or other targets for diagnostics, prognostics, drug metabolism, etc. [Mitani, Y.; Lezhava, A.; Kawai, Y.; Kikuchi, T.; Oguchi-Katayama, A.; Kogo, Y.; Itoh, M.; Miyagi, T.; Takakura, H.; Hoshi, K., et al. Rapid SNP diagnostics using asymmetric isothermal amplification and a new mismatch-suppression technology. Nat. Methods 2007, 4, 257-262, doi:10.1038/nmeth1007]. Fluorescence-based analysis showed high specificity and sensitivity, and in a multiplex fashion, was able to identify four disparate respiratory viral pathogen sequences. LFA allows for field-based application, while the fluorescence assay is highly scalable, allowing for quicker turnaround times. As others have reported [Kilic, T.; Weissleder, R.; Lee, H. Molecular and Immunological Diagnostic Tests of COVID-19: Current Status and Challenges. iScience 2020, 23, 101406, doi:10.1016/j.isci.2020.101406], the robust amplification obtained by RPA and LAMP can result in cross-contamination and special precautions are required, particularly in multi-step reaction conditions such as ours. In addition, faint bands on LFA can occur [Whitman, J. D.; Hiatt, J.; Mowery, C. T.; Shy, B. R.; Yu, R.; Yamamoto, T. N.; Rathore, U.; Goldgof, G. M.; Whitty, C.; Woo, J. M., et al. Evaluation of SARS-CoV-2 serology assays reveals a range of test performance. Nat. Biotechnol 2020, 38, 1174-1183, doi:10.1038/s41587-020-0659-0], particularly with increased exposure time mandating the inclusion of rigorous controls and readout standard operating procedures (i.e., evaluation in <5 min).

Using commercial reagents, described herein is a Cas (specifically, for example Cas-9)-based detection methodology for nucleic acid detection using lateral flow assays and fluorescence signal generation. Our approach adds to the armamentarium of testing methodologies that can be brought to bear to bridge the immunization-immunity gap.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method comprising
amplifying a target nucleotide to form a polynucleotide, wherein the amplification comprises using a first primer and a second primer, wherein the first primer comprises a label;
exposing the target nucleotide to Cas9 and a gRNA, wherein the Cas9 comprises a label, to form a target nucleotide-Cas9 complex; and
detecting the target nucleotide-Cas9 complex in a lateral flow assay, a fluorometric assay, or a colorimetric assay.

2. The method of claim 1, wherein the second primer is not labeled with biotin.

3. (canceled)

4. The method of claim 1, wherein the Cas9 comprises biotinylated Cas9.

5. The method of claim 1, wherein amplifying the nucleotide comprises recombinase polymerase amplification (RPA).

6. (canceled)

7. The method of claim 1, wherein the method further comprises exposing the target nucleotide to Cas9 and a gRNA in the presence of competitor DNA.

8-10. (canceled)

11. The method of claim 7, wherein the competitor DNA is present in an amount of at least 1 fold, at least 2 fold, at least 10 fold, at least 100 fold, at least 1,000 fold, at least 2,000 fold, or at least 10,000 fold the amount of the target nucleotide.

12. The method of claim 1, the method further comprises forming a Cas9:gRNA complex prior to exposing the target nucleotide to Cas9 and the gRNA.

13. The method of claim 1, wherein method comprises detecting the target nucleotide-Cas9 complex in a lateral flow assay, wherein the lateral flow assay comprises detecting an analyte labeled with biotin and FITC, a fluorescein amidite (FAM), or digoxigenin (DIG).

14. The method of claim 1, wherein the nucleotide to be amplified comprises RNA, DNA, or cDNA.

15. (canceled)

16. (canceled)

17. The method of claim 1, wherein the target nucleotide comprises a protospacer adjacent motif (PAM).

18. (canceled)

19. (canceled)

20. The method of claim 1, wherein the method comprises detecting SARS-Cov2.

21. The method of claim 1, wherein the method comprises detecting Influenza.

22. The method of claim 1, wherein the method comprises detecting RSV.

23. (canceled)

24. (canceled)

25. A composition comprising
a target nucleotide, wherein the target nucleotide comprises a label;
a labeled Cas9, a nuclease Cas9, a nickase Cas9, or any combination thereof;
and a gRNA.

26. The composition of claim 25, wherein the label of the target nucleotide comprises FITC, a fluorescein amidite (FAM), or digoxigenin (DIG), and wherein the labeled Cas9 comprises biotinylated Cas9.

27. (canceled)

28. (canceled)

29. The composition of claim 25, wherein the composition further comprises competitor DNA.

30-34. (canceled)

35. The composition of claim 25, wherein the target nucleotide comprises a protospacer adjacent motif (PAM).

36. (canceled)

37. The composition of claim 25, wherein the composition comprises a complex comprising the target nucleotide and the labeled Cas9.

38-41. (canceled)

42. The composition of claim 25, wherein the target nucleotide comprises a SARS-CoV-2-specific sequence.

43. The composition of claim 25, wherein the target nucleotide comprises an Influenza specific sequence.

44. The composition of claim 25, wherein the target nucleotide comprises a RSV specific sequence.

45. (canceled)

46. (canceled)
